(19) United States
(12) Patent Application Publication
(10) Pub. No.: US 2025/0263795 A1
(43) Pub. Date: Aug. 21, 2025

(54) METHODS FOR CLASSIFICATION OF TISSUE SAMPLES AS POSITIVE OR NEGATIVE FOR CANCER

(71) Applicant: Veracyte, Inc., South San Francisco, CA (US)

(72) Inventors: Giulia C. Kennedy, San Francisco, CA (US); Bonnie H. Anderson, Half Moon Bay, CA (US); Darya I. Chudova, San Jose, CA (US); Eric T. Wang, Milpitas, CA (US); Hui Wang, San Bruno, CA (US); Moraima Pagan, San Francisco, CA (US); Nusrat Rabbee, South San Francisco, CA (US); Jonathan I. Wilde, Burlingame, CA (US)

(21) Appl. No.: 19/197,756

(22) Filed: May 2, 2025

Related U.S. Application Data
(63) Continuation-in-part of application No. 18/823,253, filed on Sep. 3, 2024, now Pat. No. 12,297,503.
Application No. 18/823,253 is a continuation of application No. 17/157,876, filed on Jan. 25, 2021, now Pat. No. 12,110,554.
Application No. 17/157,876 is a continuation of application No. 14/153,219, filed on Jan. 13, 2014, now Pat. No. 10,934,587.
Application No. 14/153,219 is a continuation of application No. 13/318,751, filed on May 10, 2012, now Pat. No. 8,669,057.
(60) Provisional application No. 61/176,471, filed on May 7, 2009.

(51) Int. Cl. C12Q 1/6883; C12Q 1/6886

(52) U.S. Cl. CPC C12Q 1/6883; C12Q 1/6886; C12Q 2600/112; C12Q 2600/156; C12Q 2600/158

(57) ABSTRACT

The present invention relates to compositions, kits, and methods for molecular profiling and cancer diagnostics, including but not limited to genomic DNA markers associated with cancer. In particular, the present invention provides molecular profiles associated with thyroid cancer, methods of determining molecular profiles, and methods of analyzing results to provide a diagnosis.

### CROSS-REFERENCE

This application is a continuation-in-part of U.S. application Ser. No. 18/823,253, filed Sep. 3, 2024, which is a continuation of U.S. application Ser. No. 17/157,876, filed Jan. 25, 2021 (now U.S. Pat. No. 12,110,554, issued Oct. 8, 2024), which is a continuation of U.S. application Ser. No. 14/153,219, filed Jan. 13, 2014 (now U.S. Pat. No. 10,934,587, issued Mar. 2, 2021), which is a continuation of U.S. application Ser. No. 13/318,751, filed May 10, 2012 (now U.S. Pat. No. 8,669,057, issued Mar. 11, 2014), which is a national stage application of International Application No. PCT/US2010/034140, filed May 7, 2010, which claims the benefit of U.S. Provisional Application No. 61/176,471, filed May 7, 2009, each of which is incorporated herein by reference in its entirety.

### BACKGROUND

Thyroid cancer incidence has increased substantially in the United States in recent decades, with evidence to support both an increase in detection and a true increase in occurrence. Thyroid nodules are palpable in 5% of adults and are visualized with contemporary imaging in more than one-third of adults. Malignancy is present in only 5% to 15% of all thyroid nodules, and definitive diagnosis is achieved by surgical histopathology on resected tissue. Unfortunately, thyroid surgery is associated with discomfort, scarring, inconvenience, direct and indirect costs, potential lifelong medication, and occasional surgical complications. Efforts to exclude cancer with clinical assessment alone are admittedly imperfect, and laboratory testing of serum thyroid stimulating hormone levels and thyroid imaging with radionuclides or ultrasonography identify benignity with high confidence in only 4% to 26% of nodules. Forty years ago, the application of cytology to thyroid nodule specimens obtained by fine-needle aspiration (FNA) biopsy had a substantial effect on patient management by reducing surgery by one half and doubling the proportion of cancer among patients who underwent surgery. However, approximately one-third of thyroid nodule cytology findings today are cytologically indeterminate, with estimated risks of malignancy ranging from 5% to 30%. Consequently, approximately three quarters of patients with cytologically indeterminate thyroid nodules have been referred for surgery, even though 80% ultimately prove to have benign nodules.

### SUMMARY OF THE INVENTION

The present invention includes a method for diagnosing thyroid disease in a subject, the method comprising (a) providing a DNA sample from a subject; (b) detecting the presence of one or more polymorphisms selected from the group consisting of the polymorphisms listed in Tables 1, 3-6, 8 or lists 1-45 or their complement; and (c) determining whether said subject has or is likely to have a malignant or benign thyroid condition based on the results of step (b).

The present invention also includes a composition comprising one or more binding agents that specifically bind to the one or more polymorphisms selected from the group consisting of the polymorphisms listed in Tables 1, 3-6, 8 or lists 1-45 or their complement.

In another embodiment, the present invention includes a kit for diagnosing thyroid disease in a subject, the kit comprising: (a) at least one binding agent that specifically binds to the one or more polymorphisms selected from the group consisting of the polymorphisms listed in Tables 1, 3-6, 8 or lists 1-45 or their complement; and (b) reagents for detecting binding of said at least one binding agent to a DNA sample from a subject.

In another embodiment, the present invention includes a business method for diagnosing thyroid disease in a subject, the business method comprising: (a) diagnosing thyroid disease from a subject using the method stated above; (b) providing the results of the diagnosis to the subject, a healthcare provider, or a third party; and (c) billing said subject, healthcare provider, or third party

The present disclosure describes enhanced technologies for characterizing genomic information, including improved methods for the measurement of RNA transcriptome expression and sequencing of nuclear and mitochondrial RNAs, measurement changes in genomic copy number, including loss of heterozygosity, and the development of enhanced bioinformatics and machine learning strategies, resulting in a more robust genomic test.

An aspect of the present disclosure provides a method for processing or analyzing a tissue sample of a subject, comprising: (a) subjecting a first portion of the tissue sample to cytological analysis that indicates that the first portion of the tissue sample is cytologically indeterminate; (b) upon identifying the first portion of the tissue sample as being cytologically indeterminate, assaying by sequencing, array hybridization, or nucleic acid amplification a plurality of gene expression products from a second portion of the tissue sample to yield a first data set; (c) in a programmed computer, using a trained algorithm that comprises one or more classifiers to process the first data set from (b) to generate a classification of the second portion of the tissue sample as benign, suspicious for malignancy, or malignant, wherein the one or more classifiers comprises an ensemble classifier integrated with at least one index selected from the group consisting of: a follicular content index, a Hürthle cell index, and a Hürthle neoplasm index; and (d) outputting a report indicative of the classification of the second portion of the tissue sample as benign, suspicious for malignancy, or malignant.

In some embodiments, the plurality of gene expression products include two or more of sequences corresponding to mRNA transcripts, mitochondrial transcripts, and chromosomal loss of heterozygosity. In some embodiments, the classification of the second portion of the tissue sample as benign, suspicious for malignancy, or malignant has a specificity of at least about 60%. In some embodiments, the classification of the second portion of the tissue sample as benign, suspicious for malignancy, or malignant has a specificity of at least about 68%. In some embodiments, the classification of the second portion of the tissue sample as benign, suspicious for malignancy, or malignant has a specificity of at least about 70%. In some embodiments, the classification of the second portion of the tissue sample as benign, suspicious for malignancy, or malignant has a sensitivity of at least about 90%.

In some embodiments, the one or more classifiers comprises the ensemble classifier integrated with the follicular content index, the Hürthle cell index, and the Hürthle neoplasm index. In some embodiments, the one or more classifiers further comprises one or more upstream classifiers, wherein the one or more upstream classifiers are selected from the group consisting of: a parathyroid classifier, a medullary thyroid cancer (MTC) classifier, a variant detection classifier, and a fusion transcript detection classifier. In some embodiments, the one or more classifiers comprises a parathyroid classifier that identifies a presence or an absence of a parathyroid tissue in the second portion of the tissue sample. In some embodiments, the upon identification of the absence of the parathyroid tissue in the second portion of the tissue sample by the parathyroid classifier, the at least one classifier of the one or more classifiers generates the classification of the second portion of the tissue sample as benign, suspicious for malignancy, or malignant. In some embodiments, the the one or more classifiers comprises a medullary thyroid cancer (MTC) classifier that identifies a presence or an absence of a medullary thyroid cancer (MTC) in the second portion of the tissue sample. In some embodiments, the upon identification of the absence of the MTC in the second portion of the tissue sample by the MTC classifier, the at least one classifier of the one or more classifiers generates the classification of the second portion of the tissue sample as benign, suspicious for malignancy, or malignant. In some embodiments, the the one or more classifiers comprises a variant detection classifier that identifies a presence or an absence of a BRAF mutation in the second portion of the tissue sample. In some embodiments, the BRAF mutation is a BRAF V600E mutation. In some embodiments, the upon identification of the absence of the BRAF mutation in the second portion of the tissue sample by the variant detection classifier, the at least one classifier of the one or more classifiers generates the classification of the second portion of the tissue sample as benign, suspicious for malignancy, or malignant. In some embodiments, the one or more classifiers comprises a fusion transcript detection classifier that identifies a presence or an absence of a RET/PTC gene fusion in the second portion of the tissue sample. In some embodiments, the RET/PTC gene fusion is RET/PTC1 or RET/PTC3 gene fusion. In some embodiments, the upon identification of the absence of the RET/PTC gene fusion in the second portion of the tissue sample by the fusion transcript detection classifier, the at least one classifier of the one or more classifiers generates the classification of the second portion of the tissue sample as benign, suspicious for malignancy, or malignant. In some embodiments, the follicular content index identifies follicular content in the second portion of the tissue sample.

In some embodiments, the ensemble classifier analyzes, in the first data set, sequence information corresponding to at least 500 genes of Table 3. In some embodiments, the ensemble classifier analyzes, in the first data set, sequence information corresponding to at least 1000 genes of Table 3. In some embodiments, the ensemble classifier analyzes, in the first data set, sequence information corresponding to 1115 genes of Table 3.

In some embodiments, the method further comprising (e) upon identifying the second portion of the tissue sample as being suspicious for malignancy, or malignant (i) processing the first data set to identify one or more genetic aberrations in one or more genes listed in FIG. 12; and (ii) outputting a second report indicative of a risk of malignancy, a histological subtype, and a prognosis associated with each of one of more genetic aberration identified in the second portion of the tissue sample. In some embodiments, the one or more genetic aberrations is a DNA variant. In some embodiments, the one or more genetic aberrations is a RNA fusion. In some embodiments, the risk of malignancy characterizes the one or more genetic aberrations as (1) highly associated with malignant nodules, (2) associated with both benign and malignant nodules, or (3) has insufficient published evidence.

In some embodiments, the tissue sample is a thyroid tissue sample. In some embodiments, the tissue sample is a needle aspirate sample. In some embodiments, the needle aspirate sample is a fine needle aspirate sample. In some embodiments, the malignancy is thyroid cancer.

Another aspect of the present disclosure provides a method for processing or analyzing a tissue sample of a subject, comprising: (a) subjecting a first portion of the tissue sample to cytological analysis that indicates that the first portion of the tissue sample is cytologically indeterminate; (b) upon identifying the first portion of the tissue sample as being cytologically indeterminate, assaying by sequencing, array hybridization, or nucleic acid amplification a plurality of gene expression products from a second portion of the tissue sample to yield a first data set, wherein the plurality of gene expression products include two or more of sequences corresponding to mRNA transcripts, mitochondrial transcripts, and chromosomal loss of heterozygosity; (c) in a programmed computer, using a trained algorithm that comprises one or more classifiers to process the first data set from (b) to generate a classification of the second portion of the tissue sample as benign, suspicious for malignancy, or malignant; and (d) outputting a report indicative of the classification of the second portion of the tissue sample as benign, suspicious for malignancy, or malignant.

In some embodiments, the one or more classifiers comprises an ensemble classifier integrated with at least one index selected from the group consisting of: a follicular content index, a Hurthle cell index, and a Hurthle neoplasm index. In some embodiments, the one or more classifiers comprises an ensemble classifier integrated with a follicular content index, a Hurthle cell index, and a Hurthle neoplasm index.

In some embodiments, the classification of the second portion of the tissue sample as benign, suspicious for malignancy, or malignant has a specificity of at least about 60%. In some embodiments, the classification of the second portion of the tissue sample as benign, suspicious for malignancy, or malignant has a specificity of at least about 68%. In some embodiments, the classification of the second portion of the tissue sample as benign, suspicious for malignancy, or malignant has a specificity of at least about 70%. In some embodiments, the classification of the second portion of the tissue sample as benign, suspicious for malignancy, or malignant has a sensitivity of at least about 90%.

In some embodiments, the one or more classifiers further comprises one or more upstream classifiers, wherein the one or more upstream classifiers are selected from the group consisting of: a parathyroid classifier, a medullary thyroid cancer (MTC) classifier, a variant detection classifier, and a fusion transcript detection classifier. In some embodiments, the one or more classifiers comprises a parathyroid classifier that identifies a presence or an absence of a parathyroid tissue in the second portion of the tissue sample. In some embodiments, the upon identification of the absence of the parathyroid tissue in the second portion of the tissue sample by the parathyroid classifier, the at least one classifier of the one or more classifiers generates the classification of the second portion of the tissue sample as benign, suspicious for malignancy, or malignant. In some embodiments, the one or more classifiers comprises a medullary thyroid cancer (MTC) classifier that identifies a presence or an absence of a medullary thyroid cancer (MTC) in the second portion of the tissue sample. In some embodiments, the upon identification of the absence of the MTC in the second portion of the tissue sample by the MTC classifier, the at least one classifier of the one or more classifiers generates the classification of the second portion of the tissue sample as benign, suspicious for malignancy, or malignant. In some embodiments, the one or more classifiers comprises a variant detection classifier that identifies a presence or an absence of a BRAF mutation in the second portion of the tissue sample. In some embodiments, the BRAF mutation is a BRAF V600E mutation. In some embodiments, the upon identification of the absence of the BRAF mutation in the second portion of the tissue sample by the variant detection classifier, the at least one classifier of the one or more classifiers generates the classification of the second portion of the tissue sample as benign, suspicious for malignancy, or malignant. In some embodiments, the one or more classifiers comprises a fusion transcript detection classifier that identifies a presence or an absence of a RET/PTC gene fusion in the second portion of the tissue sample. In some embodiments, the RET/PTC gene fusion is RET/PTC1 or RET/PTC3 gene fusion. In some embodiments, the upon identification of the absence of the RET/PTC gene fusion in the second portion of the tissue sample by the fusion transcript detection classifier, the at least one classifier of the one or more classifiers generates the classification of the second portion of the tissue sample as benign, suspicious for malignancy, or malignant. In some embodiments, the follicular content index identifies follicular content in the second portion of the tissue sample.

In some embodiments, the one or more classifiers of the trained algorithm comprises an ensemble classifier, wherein the ensemble classifier analyzes, in the first data set, sequence information corresponding to at least 500 genes of Table 3. In some embodiments, the one or more classifiers of the trained algorithm comprises ensemble classifier, wherein the ensemble classifier analyzes, in the first data set, sequence information corresponding to at least 1000 genes of Table 3. In some embodiments, the one or more classifiers of the trained algorithm comprises ensemble classifier, wherein the ensemble classifier analyzes, in the first data set, sequence information corresponding to 1115 genes of Table 3.

In some embodiments, the method further comprising (e) upon identifying the second portion of the tissue sample as being suspicious for malignancy, or malignant (i) processing the first data set to identify one or more genetic aberrations in one or more genes listed in FIG. 12; and (ii) outputting a second report indicative of a risk of malignancy, a histological subtype, and a prognosis associated with each of one of more genetic aberration identified in the second portion of the tissue sample. In some embodiments, the one or more genetic aberrations is a DNA variant. The method of claim 53, wherein the one or more genetic aberrations is a RNA fusion. In some embodiments, the risk of malignancy characterizes the one or more genetic aberrations as (1) highly associated with malignant nodules, (2) associated with both benign and malignant nodules, or (3) has insufficient published evidence.

In some embodiments, the tissue sample is a thyroid tissue sample. In some embodiments, the tissue sample is a needle aspirate sample. In some embodiments, the needle aspirate sample is a fine needle aspirate sample. In some embodiments, the malignancy is thyroid cancer.

Another aspect of the present disclosure provides a method for processing or analyzing a tissue sample of a subject, comprising: (a) subjecting a first portion of the tissue sample to cytological analysis that indicates that the first portion of the sample is cytologically indeterminate; (b) upon identifying the first portion of the tissue sample as being cytologically indeterminate, assaying by sequencing, array hybridization, or nucleic acid amplification a plurality of gene expression products from a second portion of the tissue sample to yield a first data set; (c) in a programmed computer, using a trained algorithm that comprises one or more classifiers to process the first data set from (b) to generate a classification of the second portion of the tissue sample as benign, suspicious for malignancy, or malignant with a specificity of at least about 60%; and (d) outputting a report indicative of the classification of the second portion of the tissue sample as benign, suspicious for malignancy, or malignant.

In some embodiments, the one or more classifiers comprises an ensemble classifier integrated with at least one index selected from the group consisting of: a follicular content index, a Hürthle cell index, and a Hurthle neoplasm index. In some embodiments, the one or more classifiers comprises an ensemble classifier integrated with a follicular content index, a Hurthle cell index, and a Hurthle neoplasm index. In some embodiments, the plurality of gene expression products include two or more of sequences corresponding to mRNA transcripts, mitochondrial transcripts, and chromosomal loss of heterozygosity.

In some embodiments, the classification of the second portion of the tissue sample as benign, suspicious for malignancy, or malignant has a specificity of at least about 68%. In some embodiments, the classification of the second portion of the tissue sample as benign, suspicious for malignancy, or malignant has a specificity of at least about 70%. In some embodiments, the classification of the second portion of the tissue sample as benign, suspicious for malignancy, or malignant has a sensitivity of at least about 90%.

In some embodiments, the one or more classifiers further comprises one or more upstream classifiers, wherein the one or more upstream classifiers are selected from the group consisting of a parathyroid classifier, a medullary thyroid cancer (MTC) classifier, a variant detection classifier, and a fusion transcript detection classifier. In some embodiments, the one or more classifiers comprises a parathyroid classifier that identifies a presence or an absence of a parathyroid tissue in the second portion of the tissue sample. In some embodiments, upon identification of the absence of the parathyroid tissue in the second portion of the tissue sample by the parathyroid classifier, the at least one classifier of the one or more classifiers generates the classification of the second portion of the tissue sample as benign, suspicious for malignancy, or malignant. In some embodiments, the one or more classifiers comprises a medullary thyroid cancer (MTC) classifier that identifies a presence or an absence of a medullary thyroid cancer (MTC) in the second portion of the tissue sample. In some embodiments, the upon identification of the absence of the MTC in the second portion of the tissue sample by the MTC classifier, the at least one classifier of the one or more classifiers generates the classification of the second portion of the tissue sample as benign, suspicious for malignancy, or malignant. In some embodiments, the one or more classifiers comprises a variant detection classifier that identifies a presence or an absence of a BRAF mutation in the second portion of the tissue sample. In some embodiments, the BRAF mutation is a BRAF V600E mutation. In some embodiments, the upon identification of the absence of the BRAF mutation in the second portion of the tissue sample by the variant detection classifier, the at least one classifier of the one or more classifiers generates the classification of the second portion of the tissue sample as benign, suspicious for malignancy, or malignant. In some embodiments, the one or more classifiers comprises a fusion transcript detection classifier that identifies a presence or an absence of a RET/PTC gene fusion in the second portion of the tissue sample. In some embodiments, the RET/PTC gene fusion is RET/PTC1 or RET/PTC3 gene fusion. In some embodiments, the upon identification of the absence of the RET/PTC gene fusion in the second portion of the tissue sample by the fusion transcript detection classifier, the at least one classifier of the one or more classifiers generates the classification of the second portion of the tissue sample as benign, suspicious for malignancy, or malignant. In some embodiments, the follicular content index identifies follicular content in the second portion of the tissue sample.

In some embodiments, the one or more classifiers of the trained algorithm comprises an ensemble classifier, wherein the ensemble classifier analyzes, in the first data set, sequence information corresponding to at least 500 genes of Table 3. In some embodiments, the one or more classifiers of the trained algorithm comprises an ensemble classifier, wherein the ensemble classifier analyzes, in the first data set, sequence information corresponding to at least 1000 genes of Table 3. In some embodiments, the one or more classifiers of the trained algorithm comprises an ensemble classifier, wherein the ensemble classifier analyzes, in the first data set, sequence information corresponding to 1115 genes of Table 3.

In some embodiments, the method further comprising (e) upon identifying the second portion of the tissue sample as being suspicious for malignancy, or malignant (i) processing the first data set to identify one or more genetic aberrations in one or more genes listed in FIG. 12; and (ii) outputting a second report indicative of a risk of malignancy, a histological subtype, and a prognosis associated with each of one of more genetic aberration identified in the second portion of the tissue sample. In some embodiments, the one or more genetic aberrations is a DNA variant. In some embodiments, the one or more genetic aberrations is a RNA fusion. In some embodiments, the risk of malignancy characterizes the one or more genetic aberrations as (1) highly associated with malignant nodules, (2) associated with both benign and malignant nodules, or (3) has insufficient published evidence.

In some embodiments, the tissue sample is a thyroid tissue sample. In some embodiments, the tissue sample is a needle aspirate sample. In some embodiments, the needle aspirate sample is a fine needle aspirate sample. In some embodiments, the malignancy is thyroid cancer.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications and patent applications mentioned in this specification are herein incorporated by reference to the same extent as if each individual publication or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is an illustration of Afirma gene sequencing classifier (“GSC”) system.

FIG. 2 illustrates Standard for Reporting of Diagnostic Accuracy Studies diagram of sample flow through the study.

FIG. 3 illustrates Afirma Genomic Sequencing Classifier (“GSC”) performance across differing risk populations.

FIG. 4 illustrates that Afirma GSC significantly improves specificity and high sensitivity.

FIG. 5 illustrates that in a comparison between Afirma GEC versus Afirma GSC, Afirma GSC shows significantly more benign results.

FIG. 6 illustrates treatment recommendations based on the results of Afirma GSC.

FIG. 7 illustrates that in a performance comparison between Afirma GEC versus Afirma GSC, GSC has a higher benign rate and PPV.

FIG. 8 illustrates analytical performance of Xpression Atlas.

FIG. 9 illustrates the diagnostic overview including Afirma GSC and Xpression Atlas.

FIG. 10 illustrates an example of an Xpression Atlas result.

FIG. 11 shows a computer system that is programmed or otherwise configured to implement methods provided herein.

FIG. 12 is a table listing certain genes identified as contributing to cancer diagnosis by molecular profiling.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “subject,” as used herein, generally refers to any animal or living organism. Animals can be mammals, such as humans, non-human primates, rodents such as mice and rats, dogs, cats, pigs, sheep, rabbits, and others. Animals can be fish, reptiles, or others. Animals can be neonatal, infant, adolescent, or adult animals. Humans can be more than about 1, 2, 5, 10, 20, 30, 40, 50, 60, 65, 70, 75, or about 80 years of age. The subject may have or be suspected of having a disease, such as cancer. The subject may be a patient, such as a patient being treated for a disease, such as a cancer patient. The subject may be predisposed to a risk of developing a disease such as cancer. The subject may be in remission from a disease, such as a cancer patient. The subject may be healthy.

The term “disease,” as used herein, generally refers to any abnormal or pathologic condition that affects a subject. Examples of a disease include cancer, such as, for example, thyroid cancer, parathyroid cancer, lung cancer, skin cancer, and others. The disease may be treatable or non-treatable. The disease may be terminal or non-terminal. The disease can be a result of inherited genes, environmental exposures, or any combination thereof. The disease can be cancer, a genetic disease, a proliferative disorder, or others as described herein.

The term “sequence variant,” “sequence variation,” “sequence alteration” or “allelic variant,” as used herein, generally refer to a specific change or variation in relation to a reference sequence, such as a genomic deoxyribonucleic acid (DNA) reference sequence, a coding DNA reference sequence, or a protein reference sequence, or others. The reference DNA sequence can be obtained from a reference database. A sequence variant may affect function. A sequence variant may not affect function. A sequence variant can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a sequence variation can include a substitution, a deletion, an insertion, an inversion, or a conversion in one or more nucleotides or one or more amino acids. A sequence variant may be a point mutation. A sequence variant may be a fusion gene. A fusion pair or a fusion gene may result from a sequence variant, such as a translocation, an interstitial deletion, a chromosomal inversion, or any combination thereof. A sequence variation can constitute variability in the number of repeated sequences, such as triplications, quadruplications, or others. For example, a sequence variation can be an increase or a decrease in a copy number associated with a given sequence (i.e., copy number variation, or CNV). A sequence variation can include two or more sequence changes in different alleles or two or more sequence changes in one allele. A sequence variation can include two different nucleotides at one position in one allele, such as a mosaic. A sequence variation can include two different nucleotides at one position in one allele, such as a chimeric. A sequence variant may be present in a malignant tissue. A sequence variant may be present in a benign tissue. Absence of a variant may indicate that a tissue or sample is benign. As an alternative, absence of a variant may not indicate that a tissue or sample is benign.

The term “disease diagnostic,” as used herein, generally refers to diagnosing or screening for a disease, to stratify a risk of occurrence of a disease, to monitor progression or remission of a disease, to formulate a treatment regime for the disease, or any combination thereof. A disease diagnostic can include a) obtaining information from one or more tissue samples from a subject, b) making a determination about whether the subject has a particular disease based on the information or tissue sample obtained, c) stratifying the risk of occurrence of the disease in the subject, d) confirming whether a subject has the disease, is developing the disease, or is in disease remission, or any combination thereof. The disease diagnostic may inform a particular treatment or therapeutic intervention for the disease. The disease diagnostic may also provide a score indicating for example, the severity or grade of a disease such as cancer, or the likelihood of an accurate diagnosis, such as via a p-value, a corrected p-value, or a statistical confidence indicator. The disease diagnostic may also indicate a particular type of a disease. For example, a disease diagnostic for thyroid cancer may indicate a subtype such as follicular adenoma (FA), nodular hyperplasia (NHP), lymphocytic thyroiditis (LCT), Hürthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), anaplastic thyroid carcinoma (ATC), renal carcinoma (RCC), breast carcinoma (BCA), melanoma (MMN), B cell lymphoma (BCL), parathyroid (PTA), or hyperplasia papillary carcinoma (HPC).

### Introduction

Some techniques for using preoperative genomic information for thyroid nodule differential diagnosis may involve use messenger RNA (“mRNA”) transcript expression levels to categorize cytologically indeterminate FNAs as either benign or suspicious. Altered messenger RNA expression can occur for several reasons, including complex upstream interactions that occur because of sequence changes in key core genes or in relevant peripheral genes, the effect of epigenetic changes that occur without DNA sequence alterations, and both internal and external modifiers, such as inflammation and lifestyle or environment. Previously, in a cohort with a 24% prevalence of malignancy, a genome expression classifier (“GEC”) accurately identified 90% of malignancies (i.e., sensitivity) and 52% of benign nodules (i.e., specificity) with indeterminate Bethesda III or IV cytology. It intentionally favored high sensitivity over specificity to ensure the accuracy and safety of a benign genomic result. In GEC, a machine learning-derived classification algorithm uses messenger RNA transcript expression levels to categorize cytologically indeterminate samples as either benign or suspicious. A test, as described in the present disclosure, that has improved specificity for identification of benign nodules and maintained high sensitivity for malignancy detection may spare even more patients from surgery with an accurate benign genomic result (negative predictive value [NPV]) and increase the cancer yield among those with a suspicious result (positive predictive value [PPV]).

The present disclosure describes enhanced technologies for characterizing genomic information, including improved methods for the measurement of RNA transcriptome expression and sequencing of nuclear and mitochondrial RNAs, measurement changes in genomic copy number, including loss of heterozygosity, and the development of enhanced bioinformatics and machine learning strategies, resulting in a more robust genomic test.

### Methods for Generating Classification for Tissue Samples for a Disease

The present disclosure provides methods for processing or analyzing a tissue sample of a subject to generate a classification of tissue sample as benign, suspicious for malignancy, or malignant. Such methods may comprise obtaining a plurality of gene expression products from a cytologically indeterminate tissue sample and using an algorithm to analyze the gene expression products to classify the tissue samples as benign, suspicious for malignancy, or malignant. In some cases, a plurality of gene expression products comprises sequences corresponding to mRNA transcripts, mitochondrial transcripts, chromosomal loss of heterozygosity, DNA variants and/or fusion transcripts. In some examples, the method uses a trained algorithm that comprises one or more classifiers and is implemented by one or more programmed computer processors to analyze the expression gene products to generate a classification of tissue sample as benign, suspicious for malignancy, or malignant. The algorithm may be a trained algorithm (e.g., an algorithm that is trained on at least 10, 200, 100 or 500 reference samples). References samples may be obtained from subjects having been diagnosed with the disease or from healthy subjects. The trained algorithm may analyze the sequence information of expression gene products corresponding to about 10,000 genes. The trained algorithm may analyze the sequence information of expression gene products corresponding to at least 500 genes of Table 3. The trained algorithm may analyze the sequence information of expression gene products corresponding to at least 600 genes of Table 3. The trained algorithm may analyze the sequence information of expression gene products corresponding to at least 700 genes of Table 3. The trained algorithm may analyze the sequence information of expression gene products corresponding to at least 800 genes of Table 3. The trained algorithm may analyze the sequence information of expression gene products corresponding to at least 900 genes of Table 3. The trained algorithm may analyze the sequence information of expression gene products corresponding to at least 1000 genes of Table 3. The trained algorithm may analyze the sequence information of expression gene products corresponding to at least 1100 genes of Table 3. The trained algorithm may analyze the sequence information of expression gene products corresponding to at least 1200 genes of Table 3.

As set forth in the present disclosure, an expression level of one or more genes of gene expression products can be obtained by assaying for an expression level. Assaying may comprise array hybridization, nucleic acid sequencing, nucleic acid amplification, or others. Assaying may comprise sequencing, such as DNA or RNA sequencing. Such sequencing may be by next generation (NextGen) sequencing, such as high throughput sequencing or whole genome sequencing (e.g., Illumina). Such sequencing may include enrichment. Assaying may comprise reverse transcription polymerase chain reaction (PCR). Assaying may utilize markers, such as primers, that are selected for each of the one or more genes of the first or second sets of genes.

Additional methods for determining gene expression levels may include but are not limited to one or more of the following: additional cytological assays, assays for specific proteins or enzyme activities, assays for specific expression products including protein or RNA or specific RNA splice variants, in situ hybridization, whole or partial genome expression analysis, microarray hybridization assays, serial analysis of gene expression (SAGE), enzyme linked immuno-absorbance assays, mass-spectrometry, immunohistochemistry, blotting, sequencing, RNA sequencing, DNA sequencing (e.g., sequencing of complementary deoxyribonucleic acid (cDNA) obtained from RNA); next generation (Next-Gen) sequencing, nanopore sequencing, pyrosequencing, or Nanostring sequencing. Gene expression product levels may be normalized to an internal standard such as total messenger ribonucleic acid (mRNA) or the expression level of a particular gene.

The methods disclosed herein may include extracting and analyzing protein or nucleic acid (RNA or DNA) from one or more samples from a subject. Nucleic acids can be extracted from the entire sample obtained or can be extracted from a portion. In some cases, the portion of the sample not subjected to nucleic acid extraction may be analyzed by cytological examination or immunohistochemistry. Methods for RNA or DNA extraction from biological samples can include for example phenol-chloroform extraction (such as guanidinium thiocyanate phenol-chloroform extraction), ethanol precipitation, spin column-based purification, or others.

The sample obtained from the subject may be cytologically ambiguous or suspicious (or indeterminate). In some cases, the sample may be suggestive of the presence of a disease. The volume of sample obtained from the subject may be small, such as about 100 microliters, 50 microliters, 10 microliters, 5 microliters, 1 microliter or less. The sample may comprise a low quantity or quality of polynucleotides, such as a tissue sample with degraded or partially degraded RNA. For example, an FNA sample may yield low quantity or quality of polynucleotides. In such examples, the RNA Integrity Number (RIN) value of the sample may be about 9.0 or less. In some examples, the RIN value may be about 6.0 or less.

### Risk of Malignancy Using Xpression Atlas

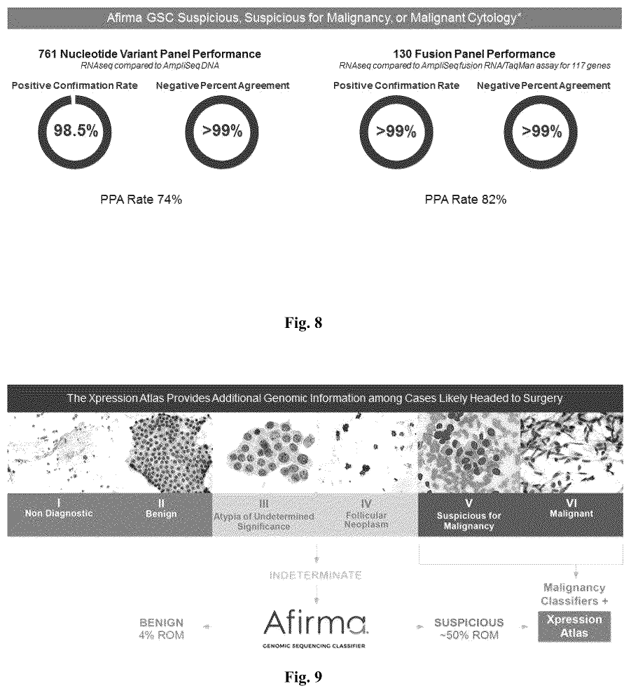

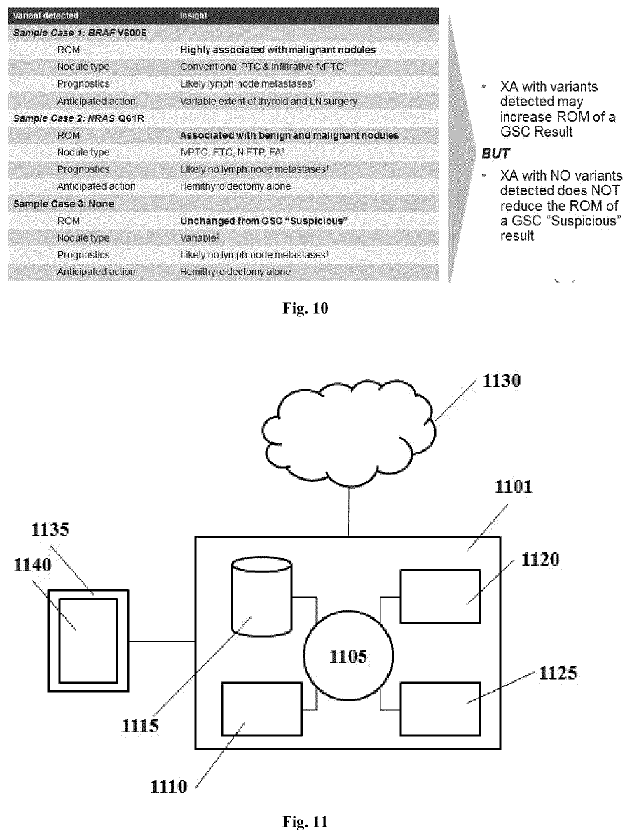

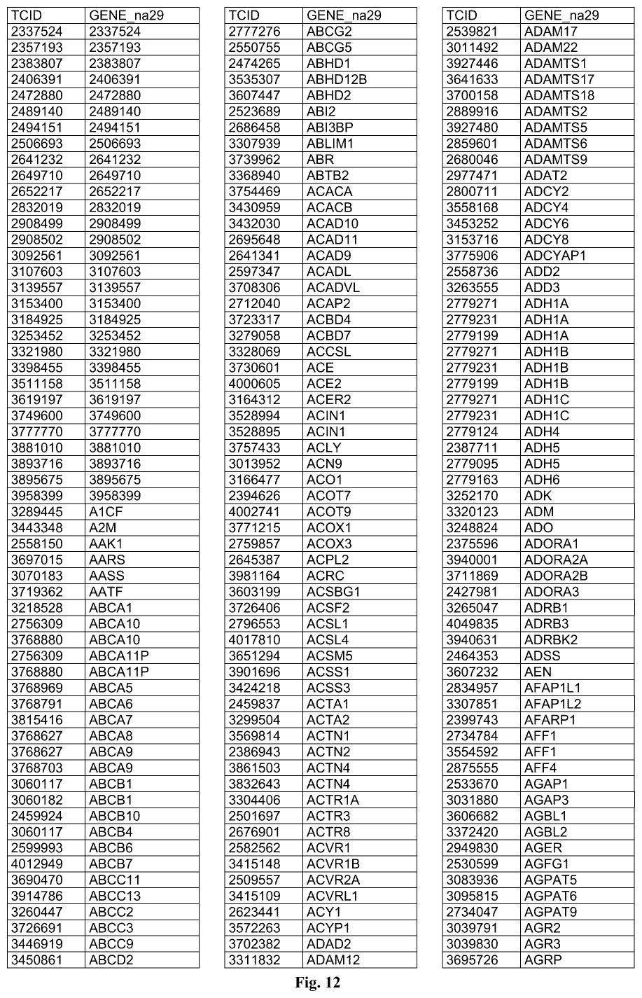

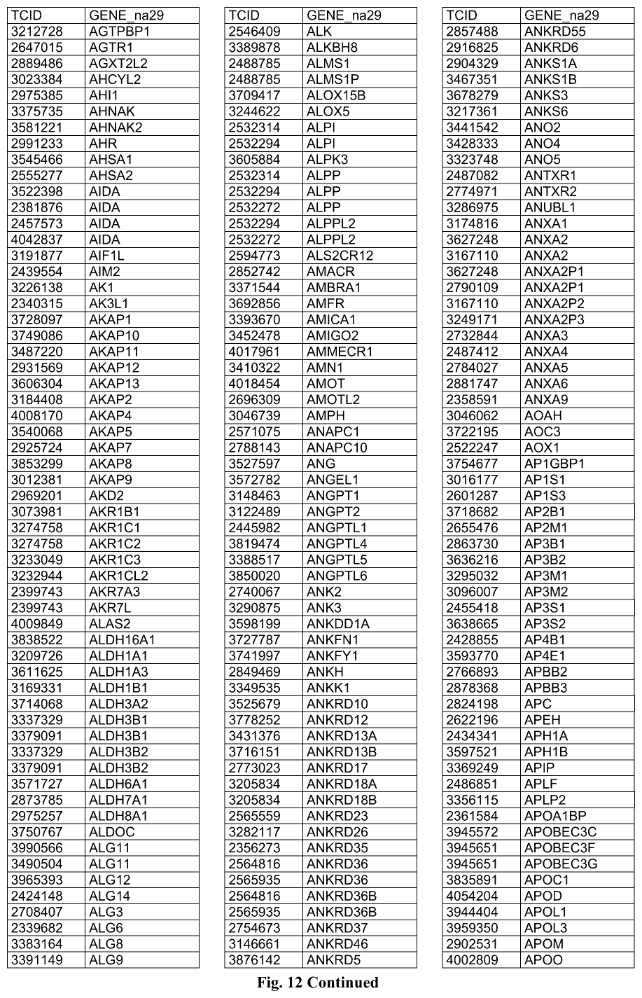

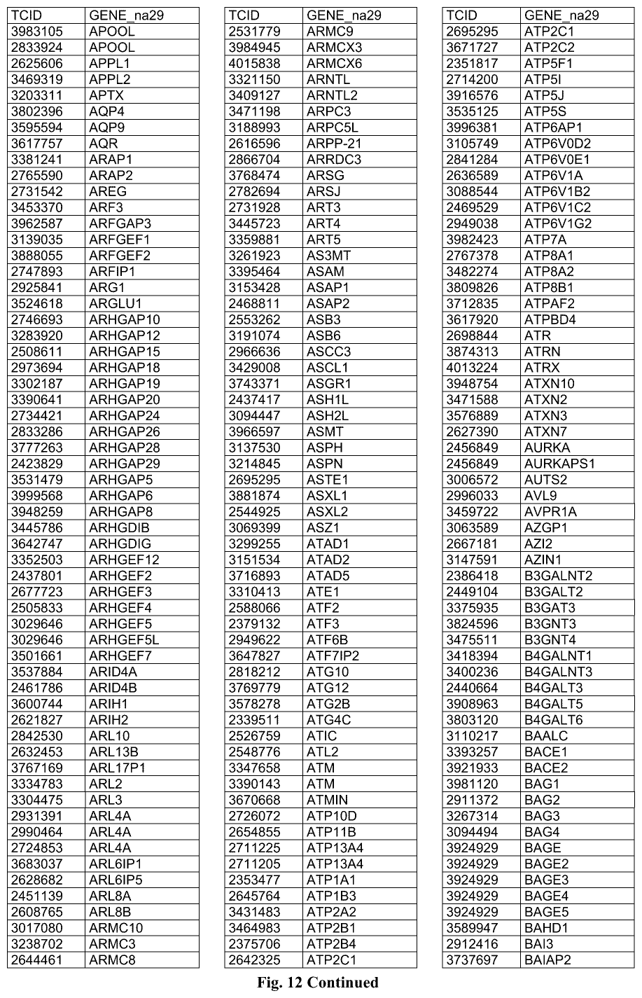

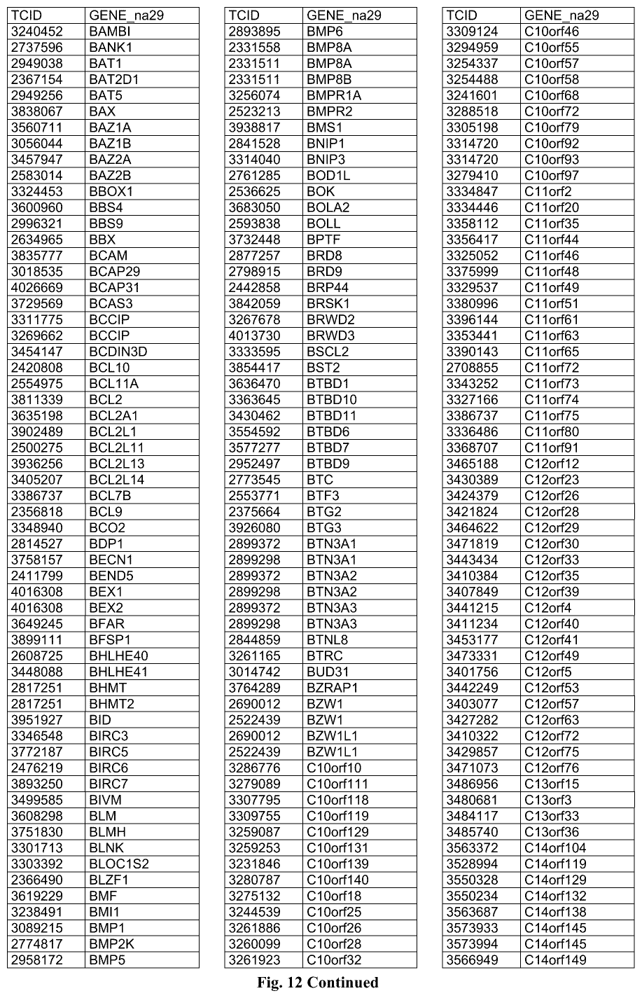

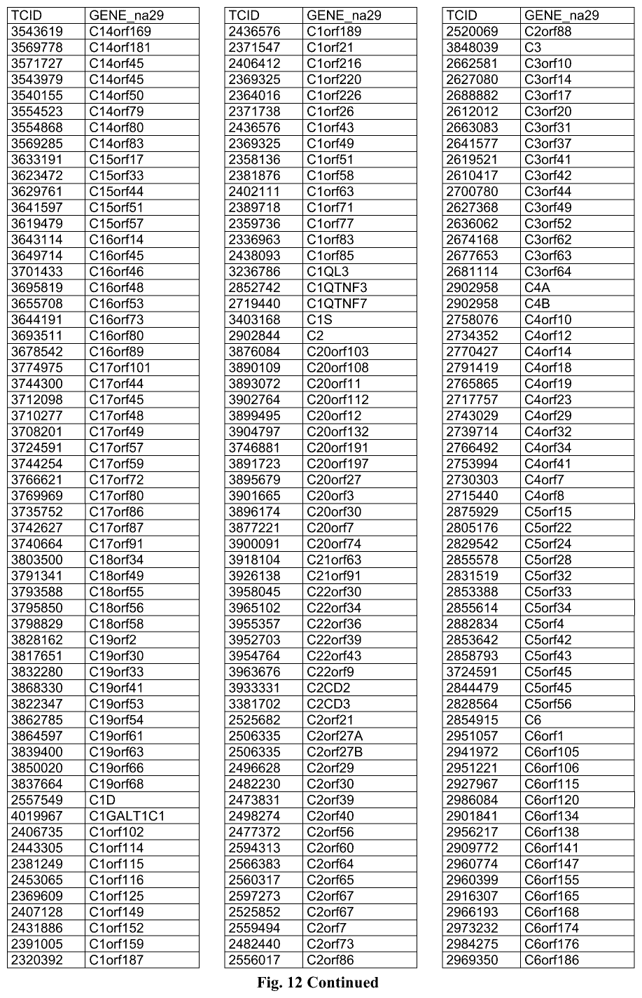

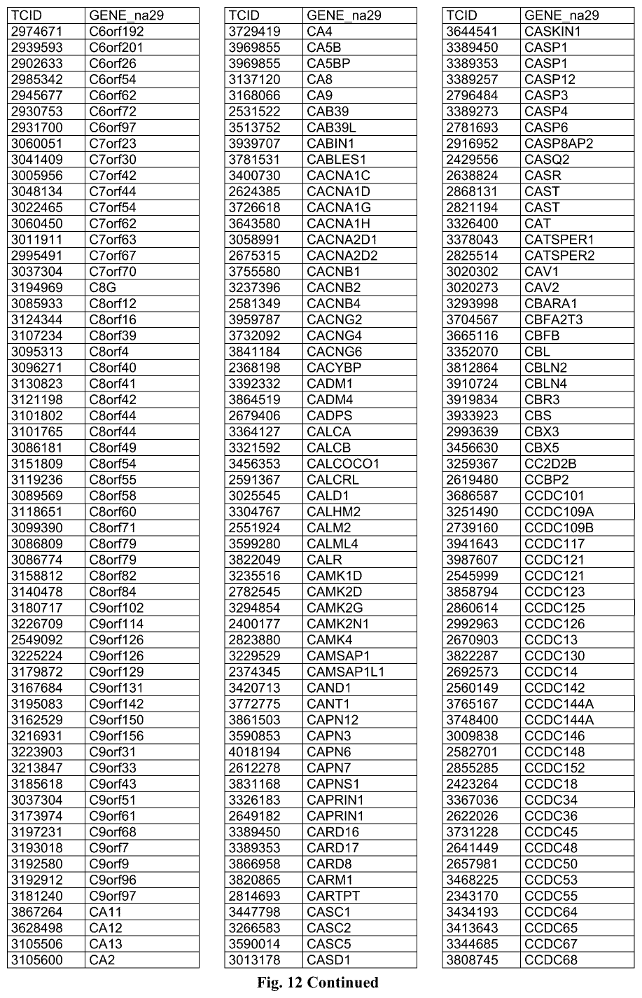

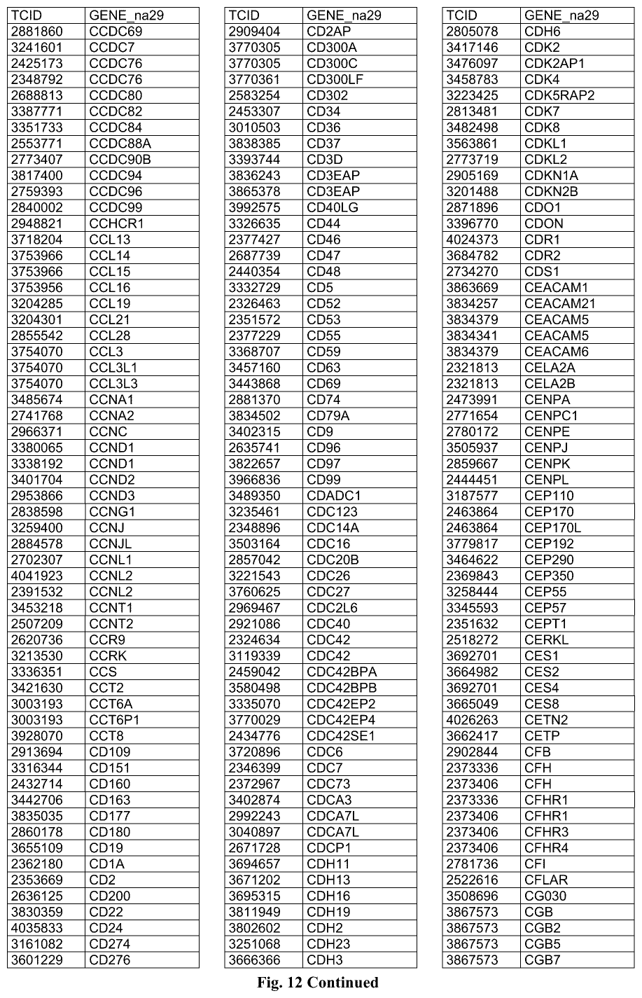

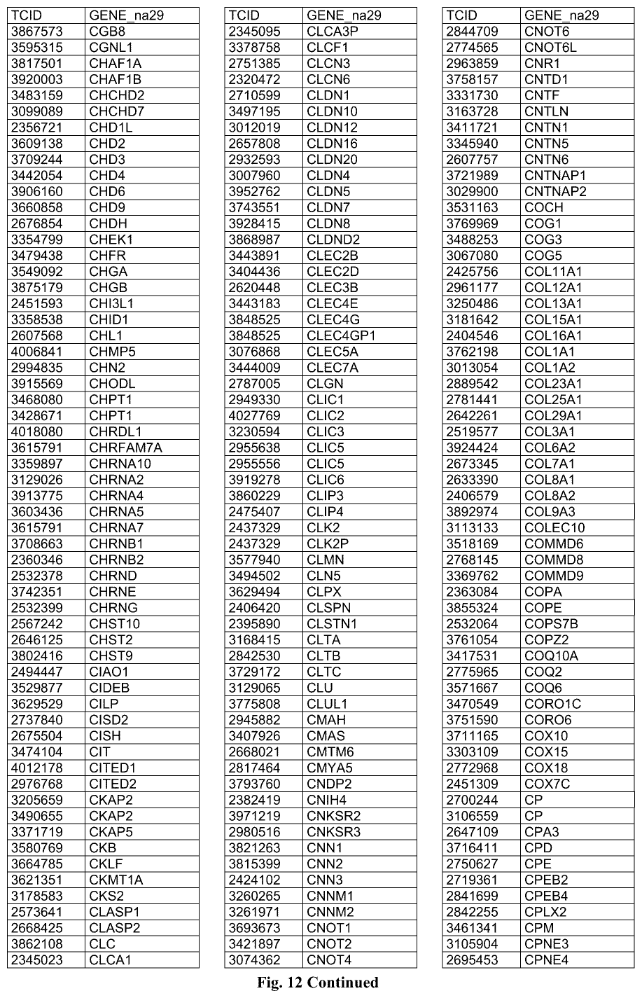

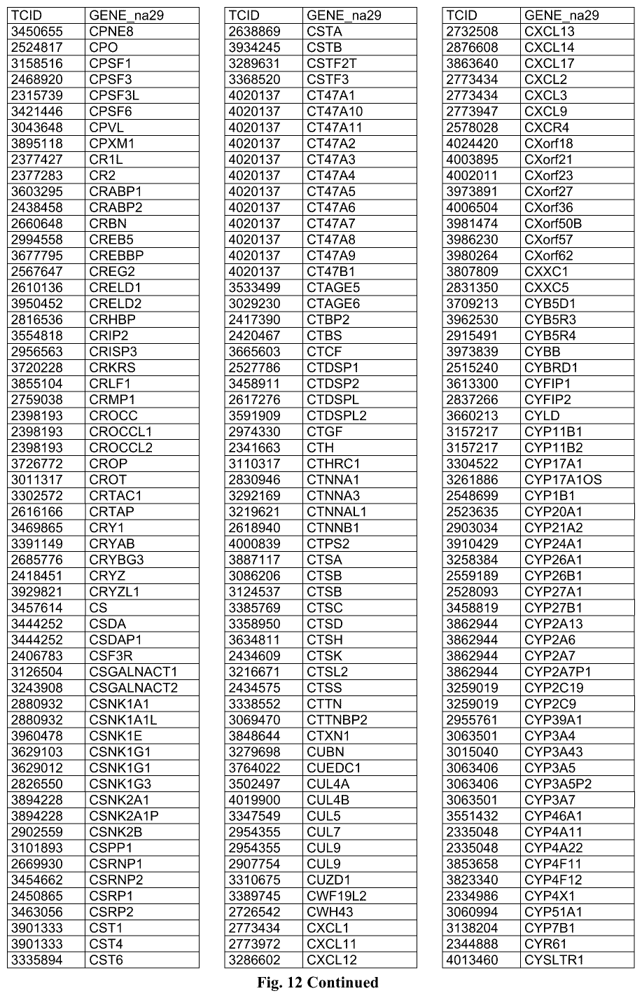

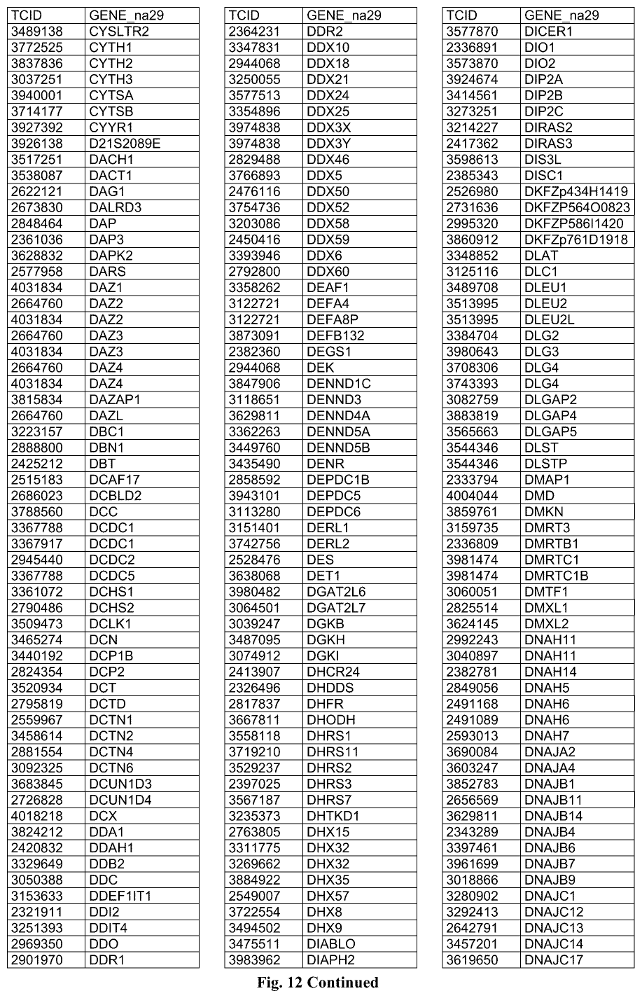

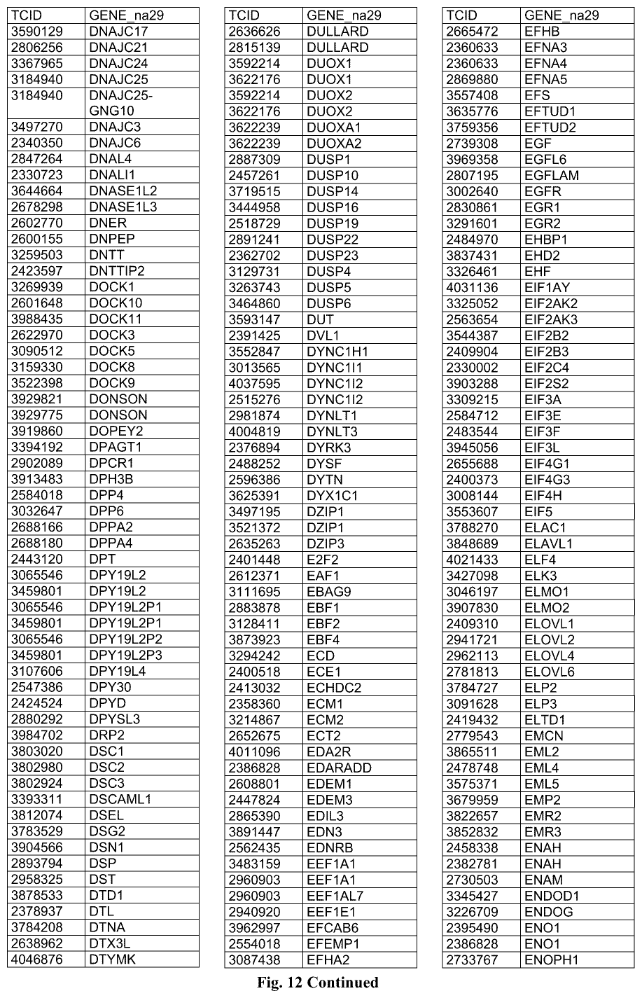

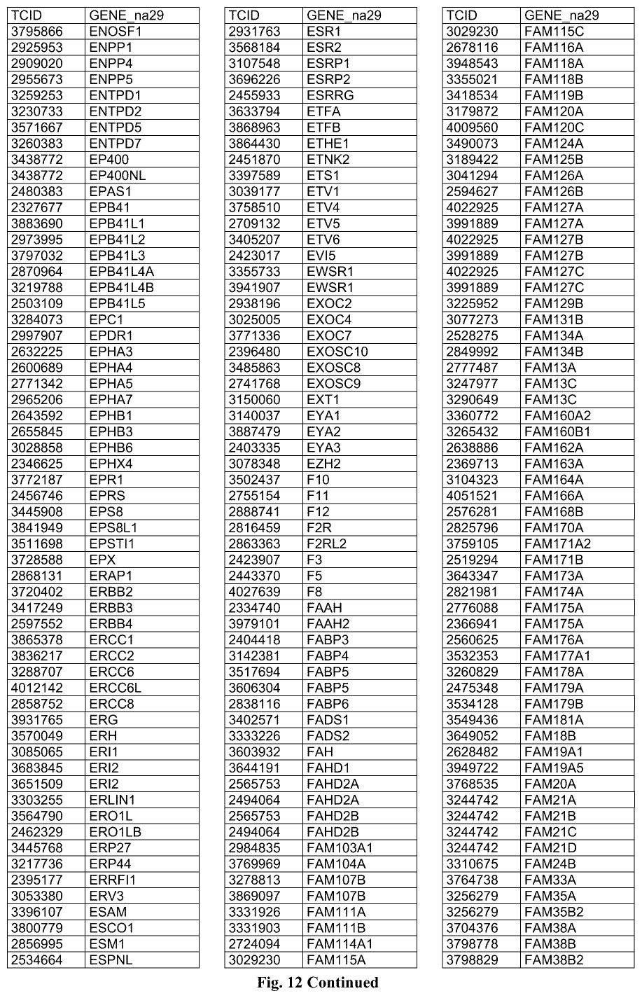

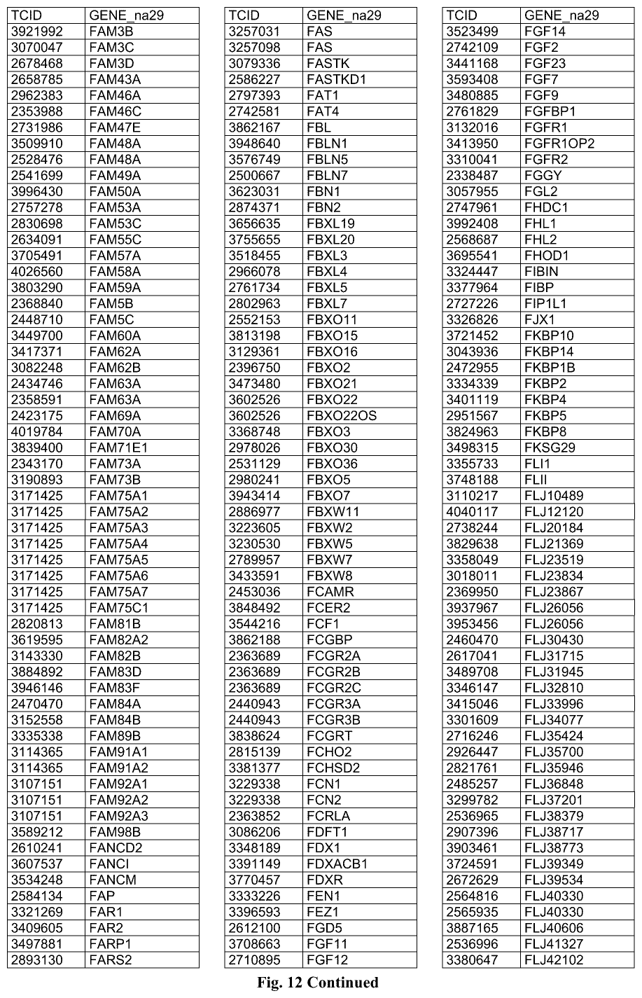

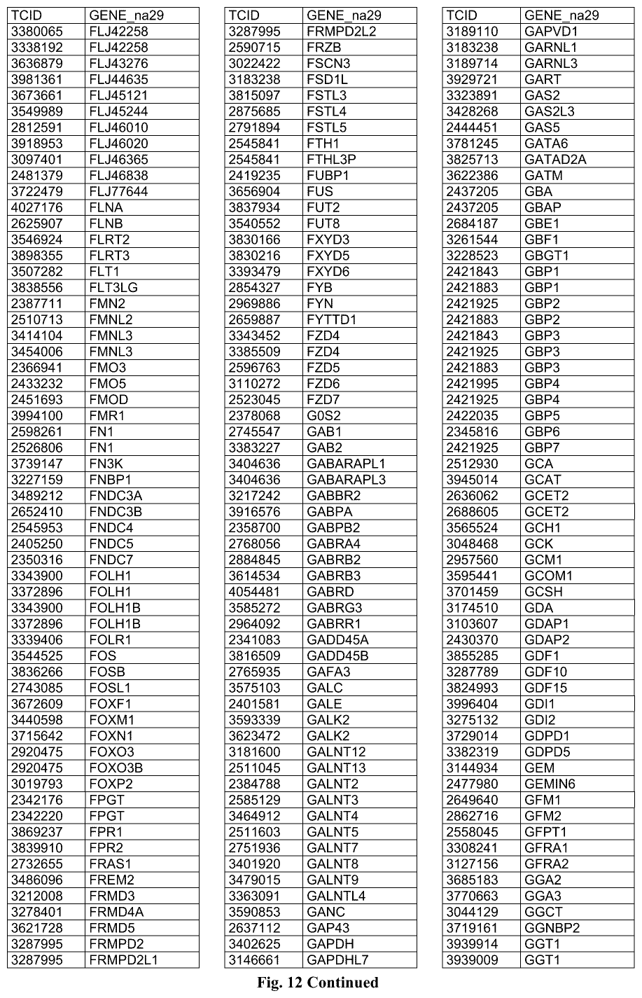

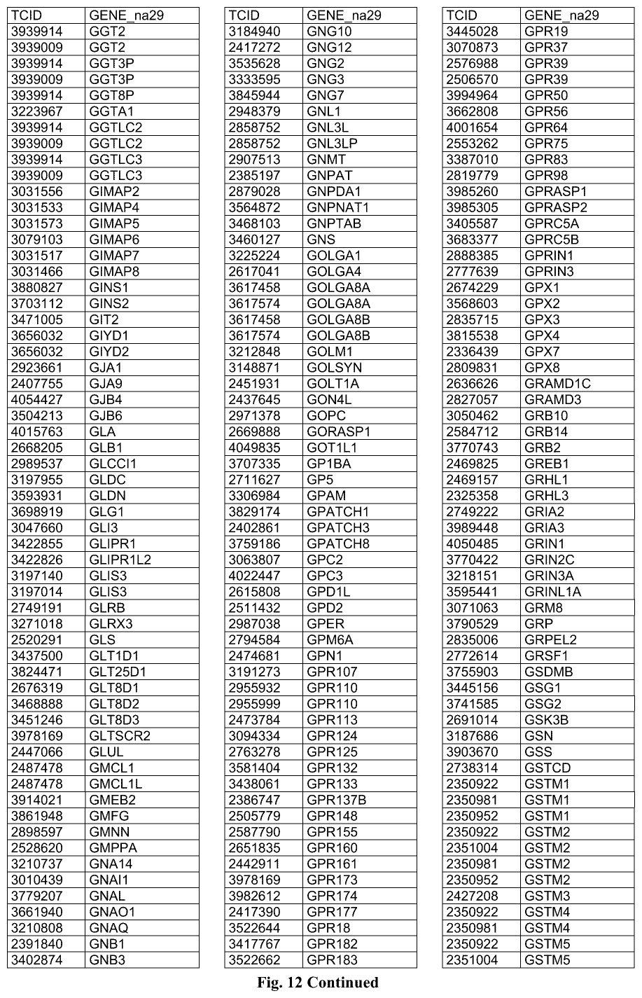

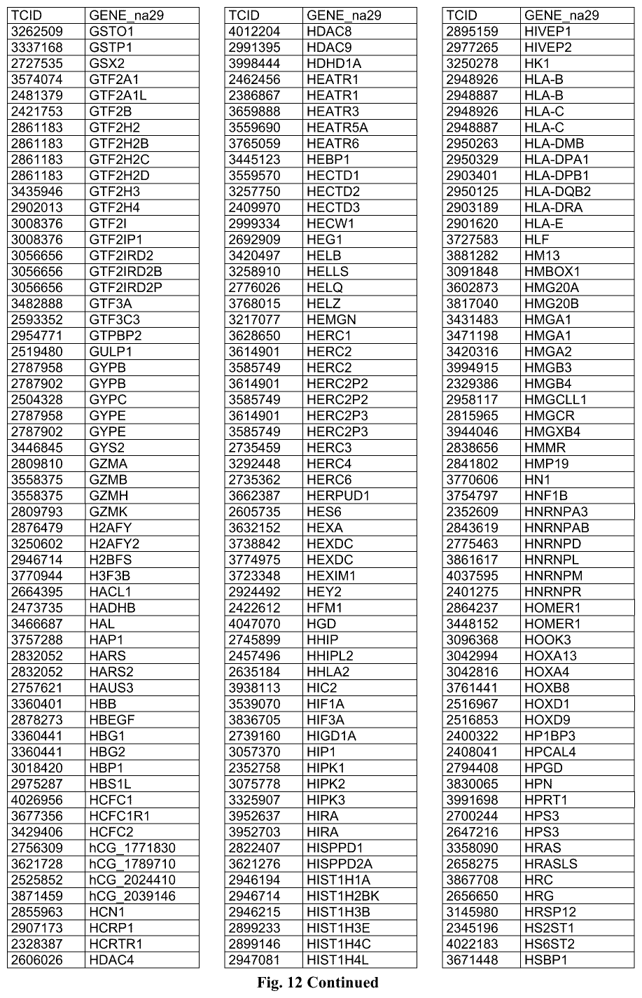

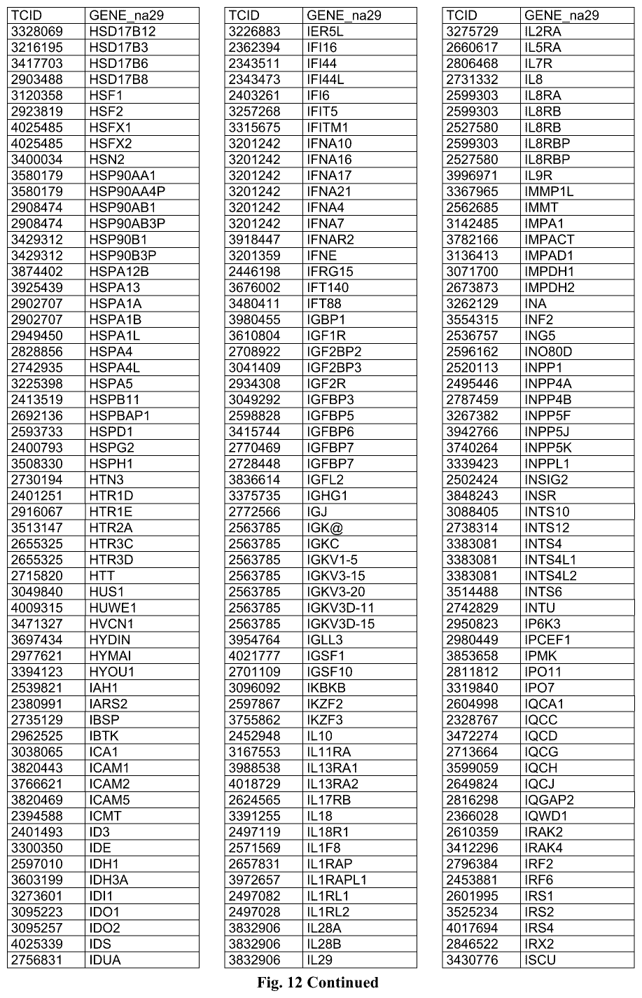

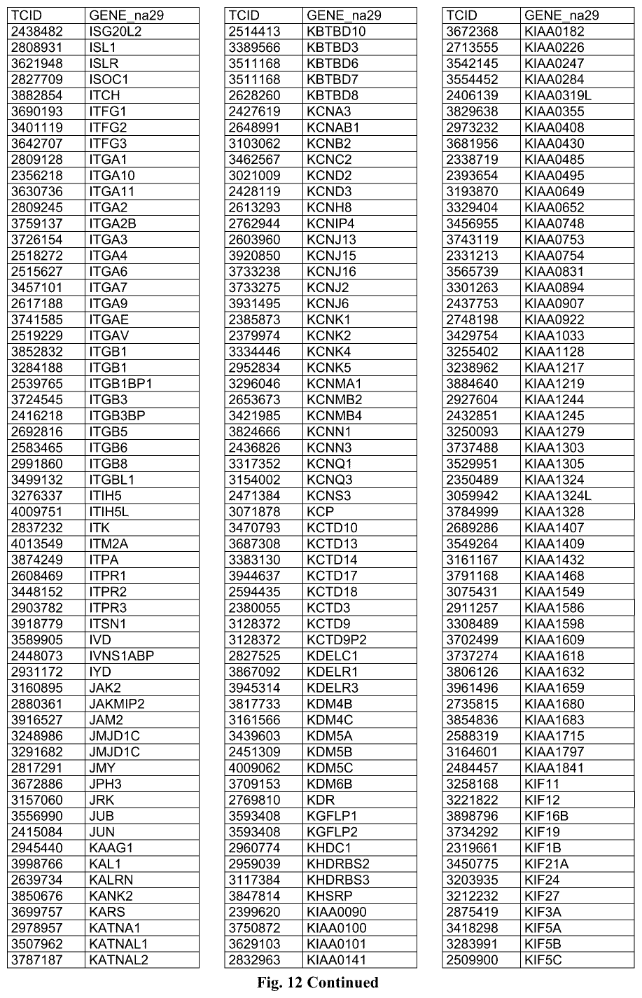

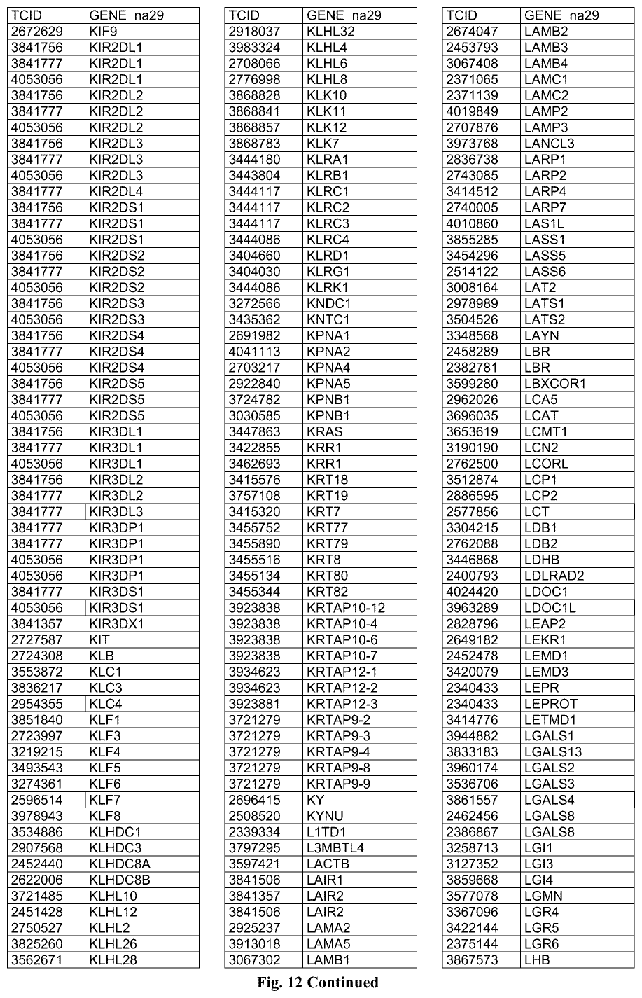

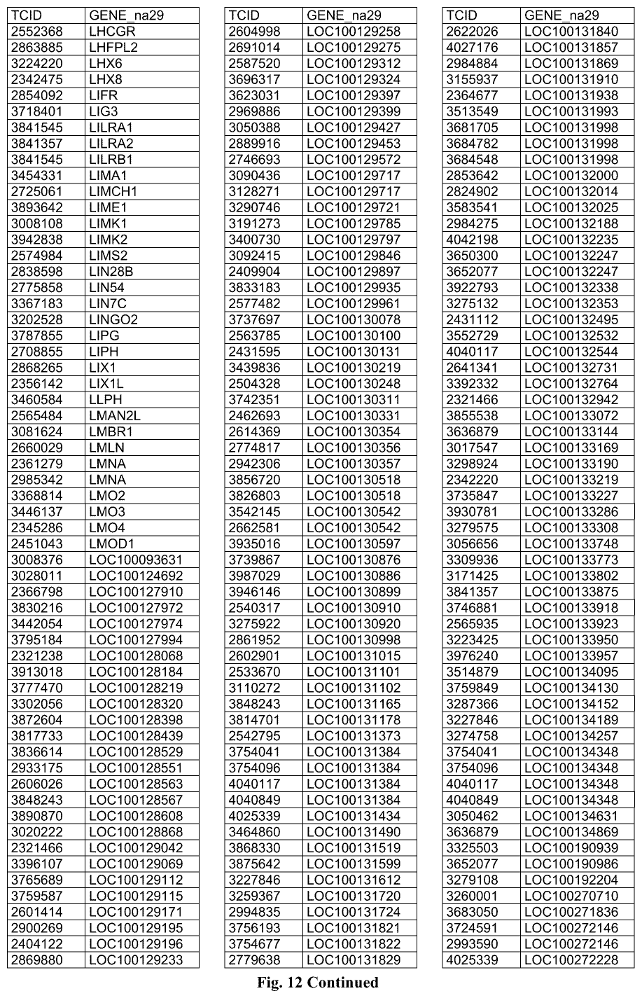

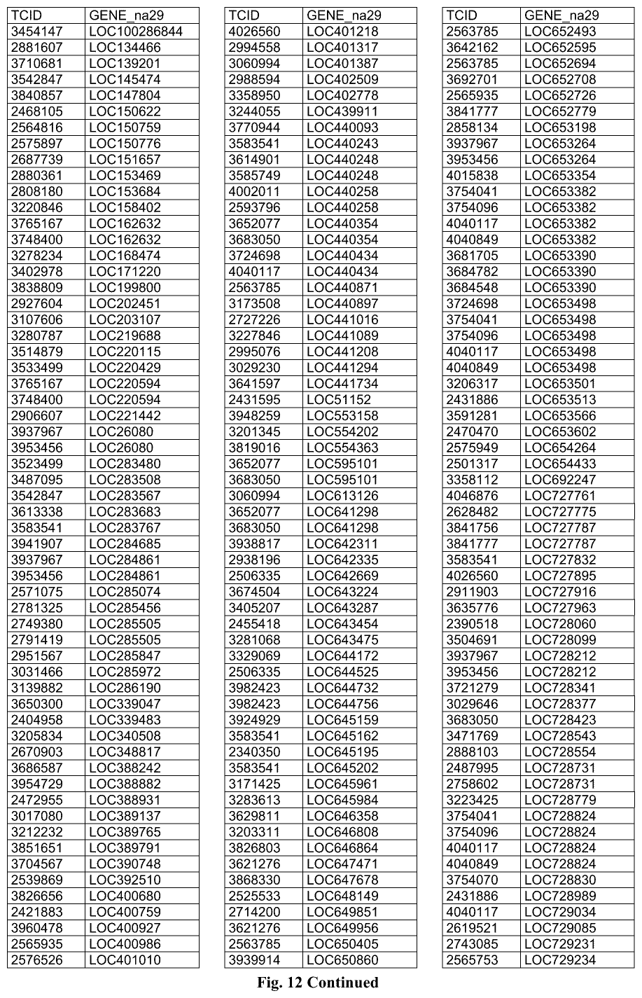

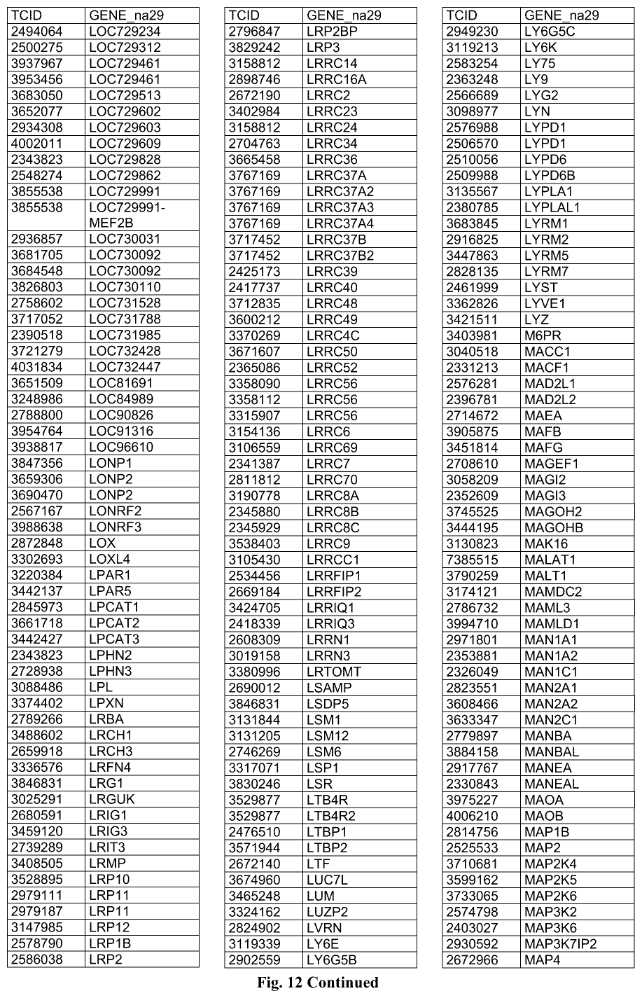

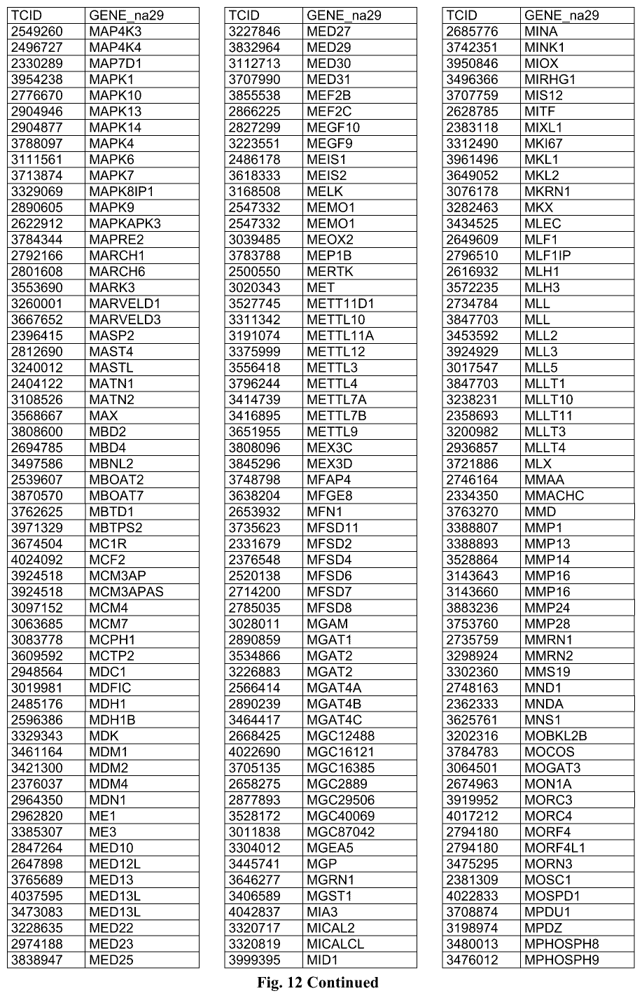

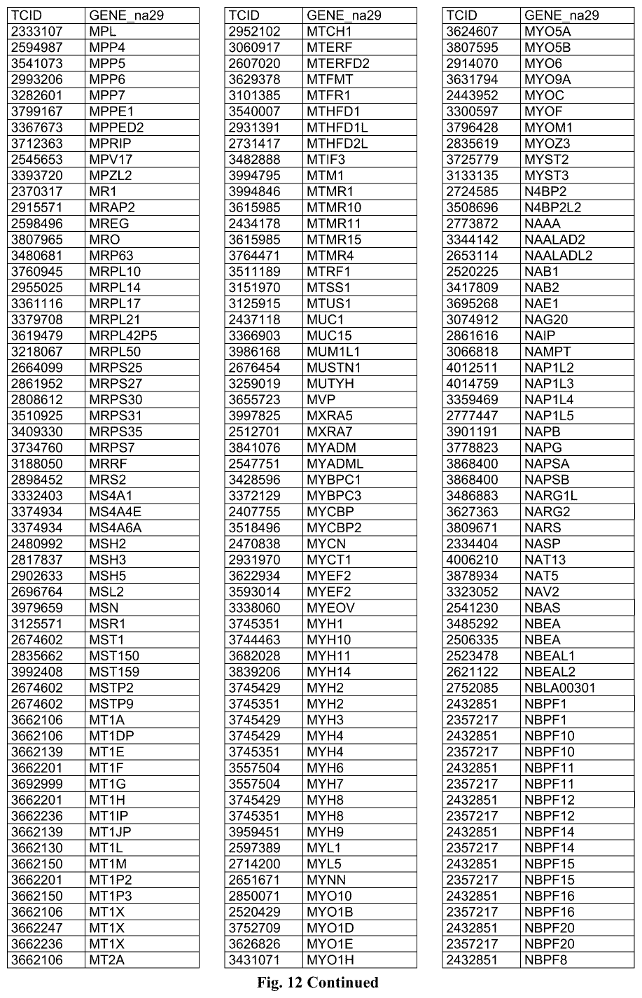

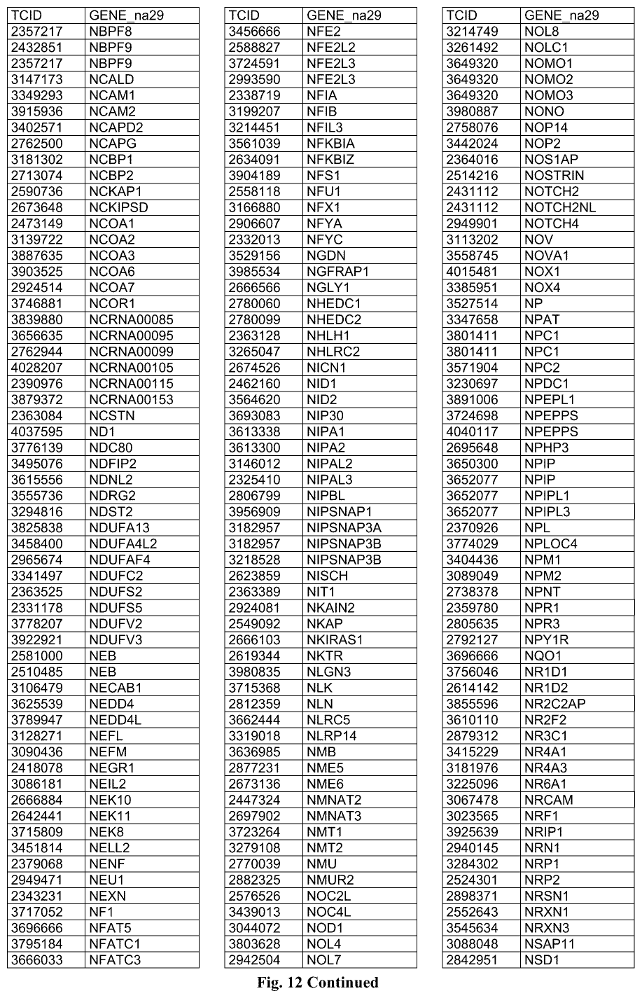

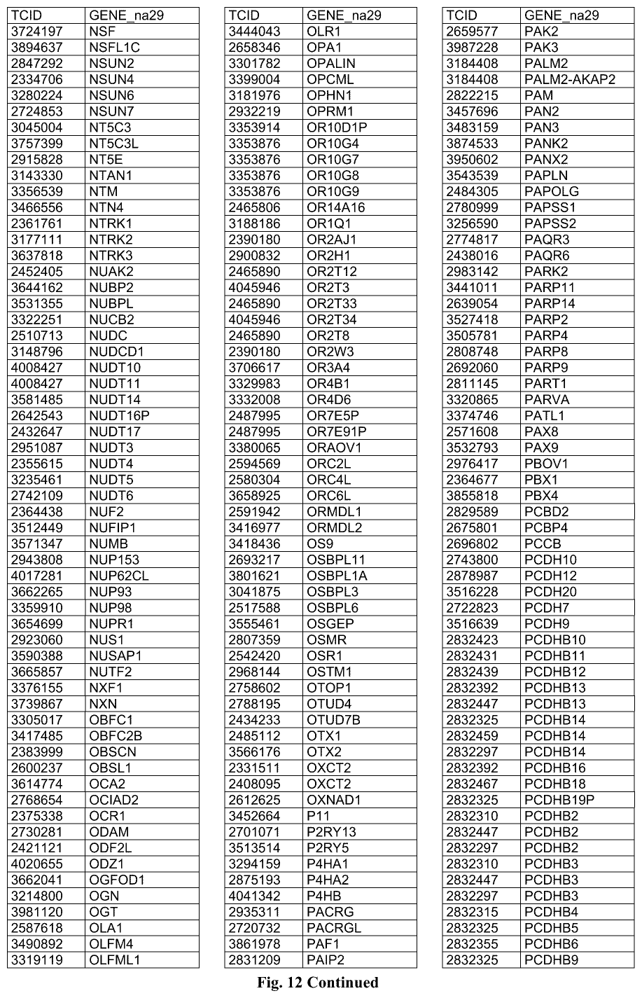

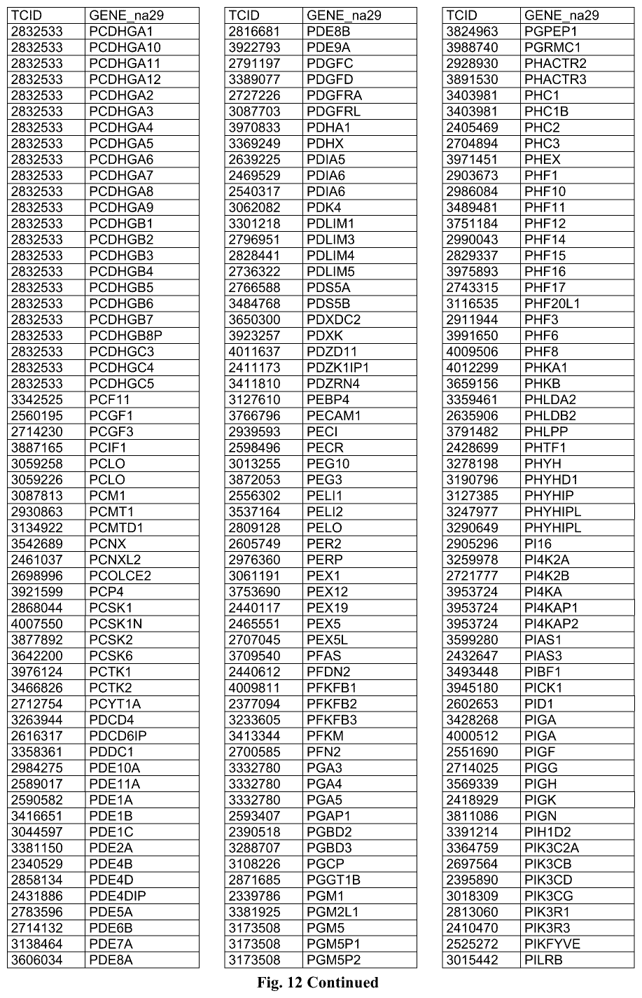

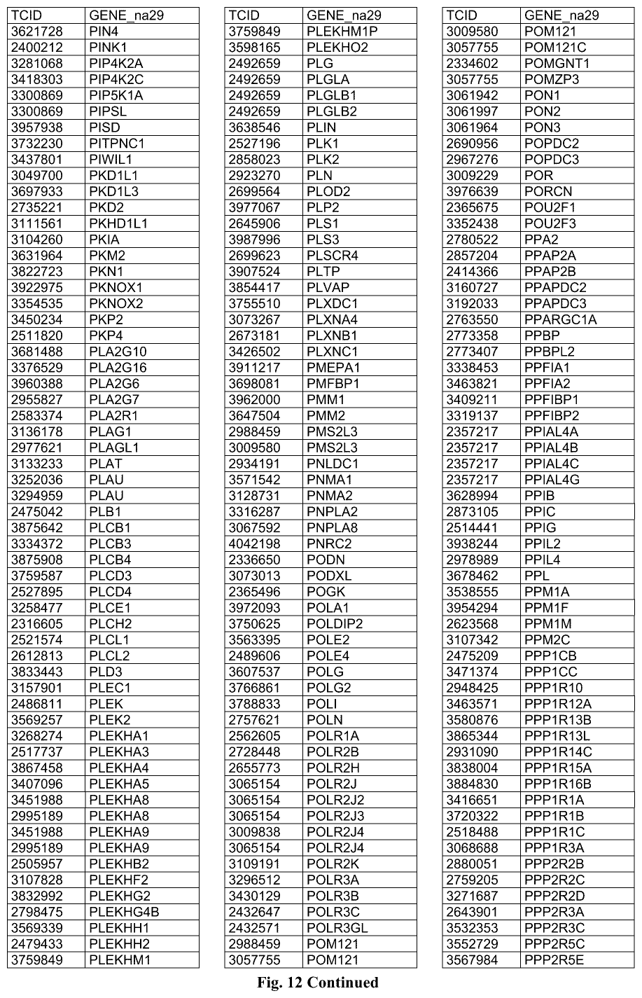

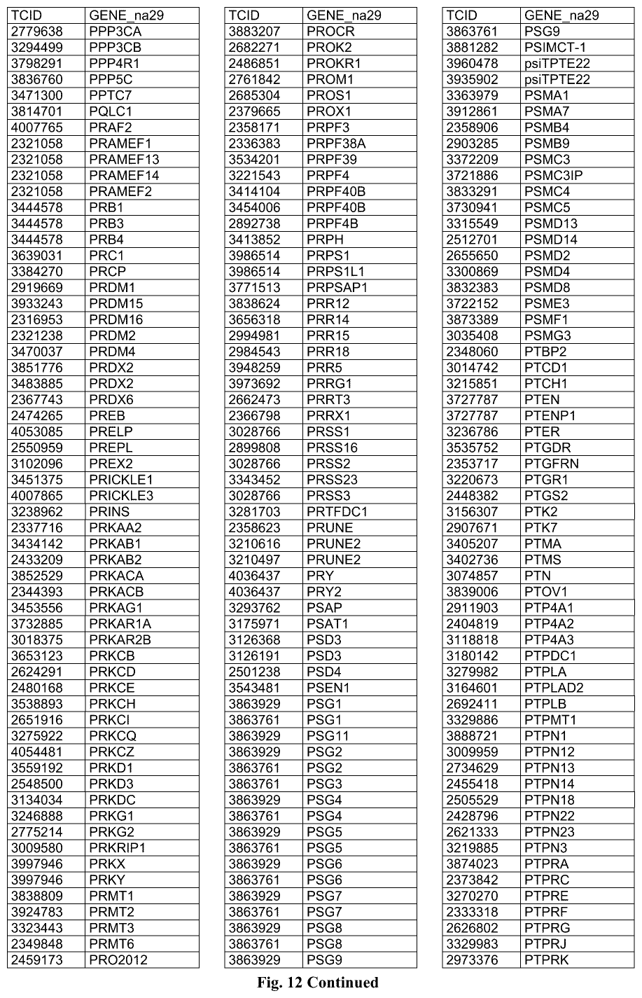

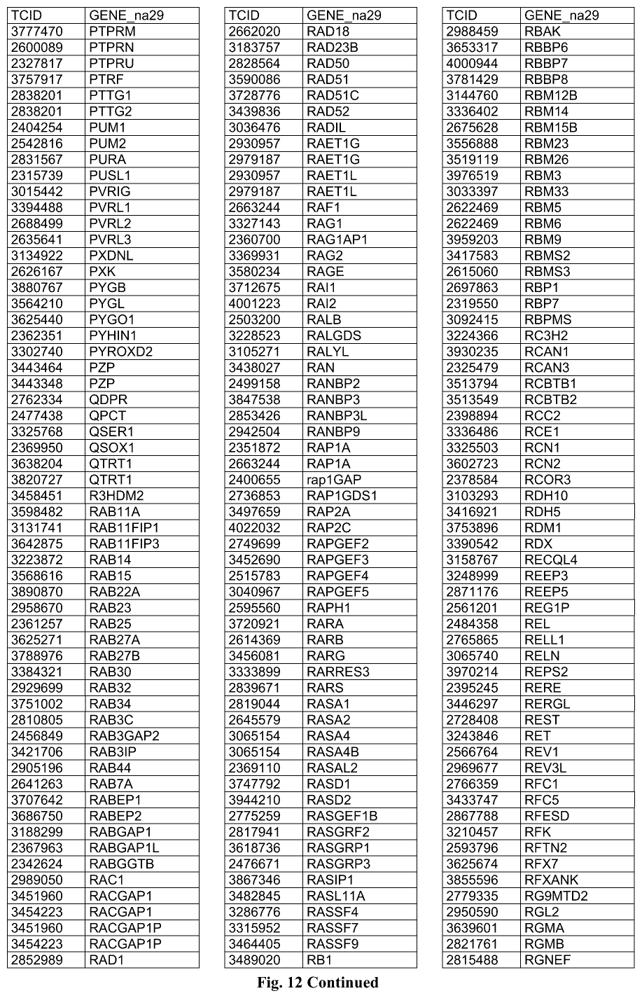

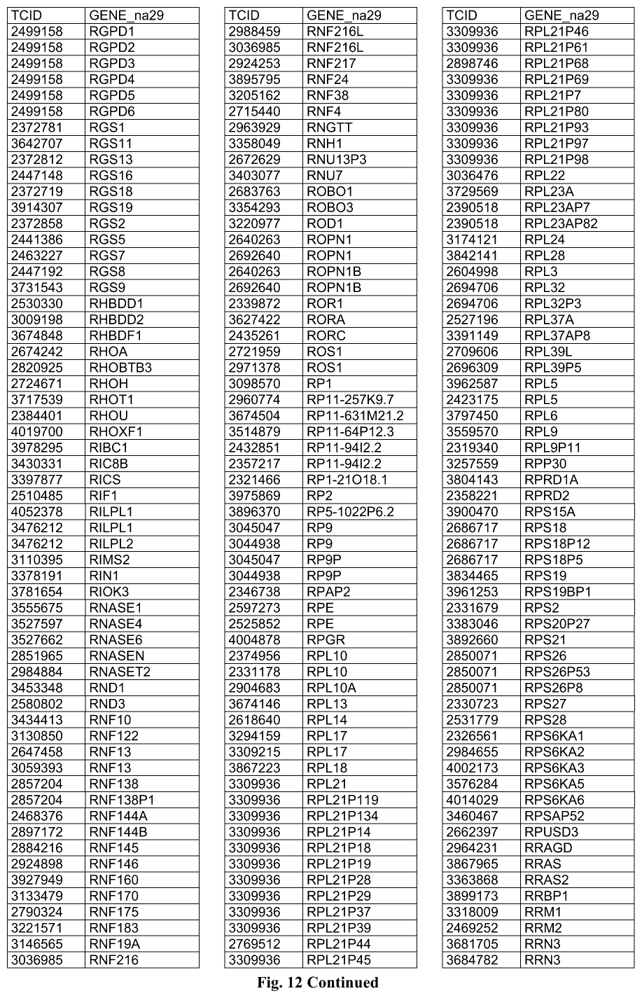

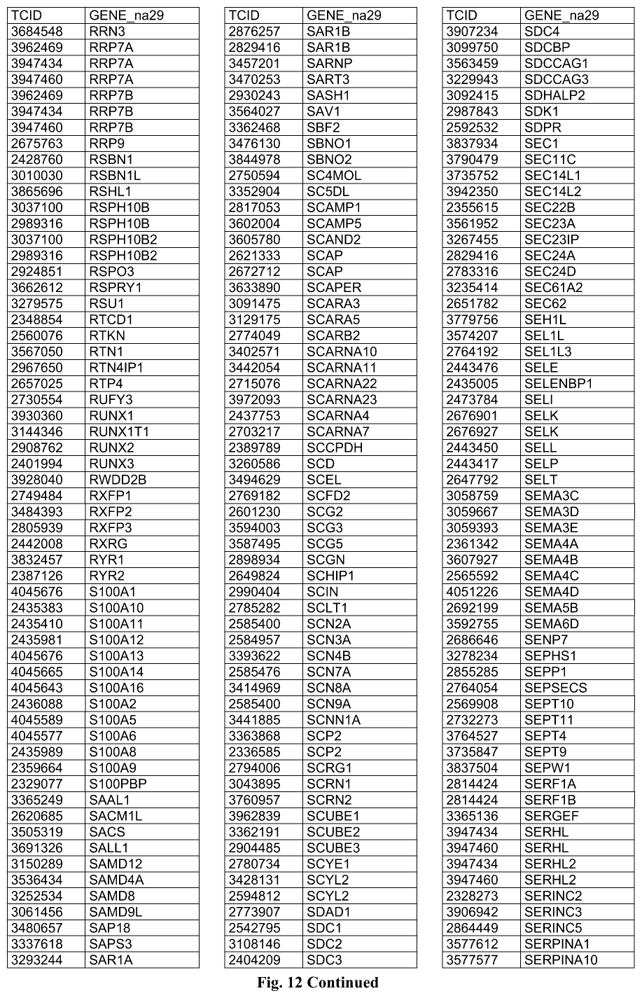

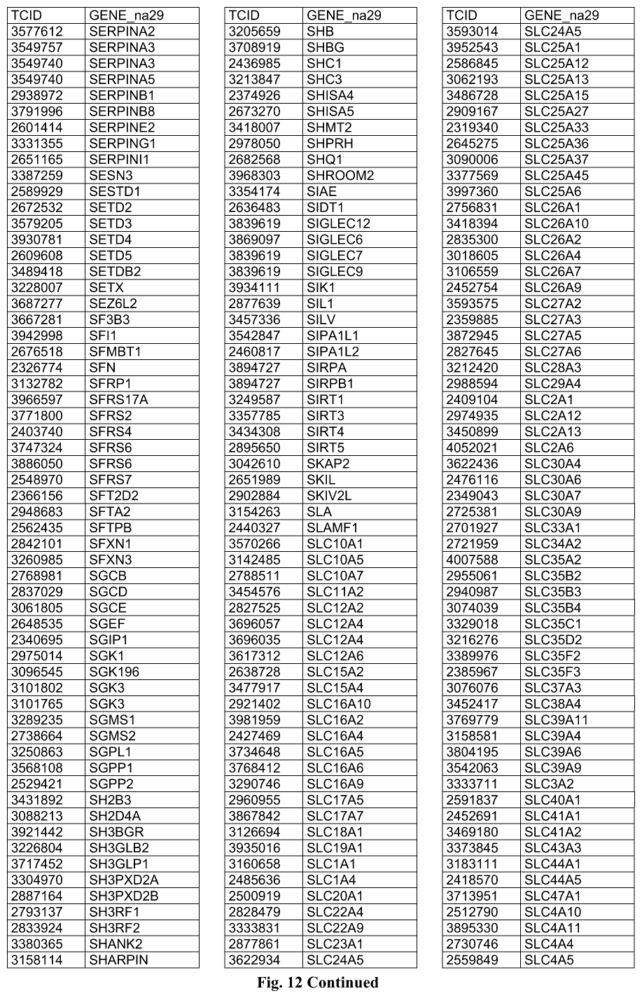

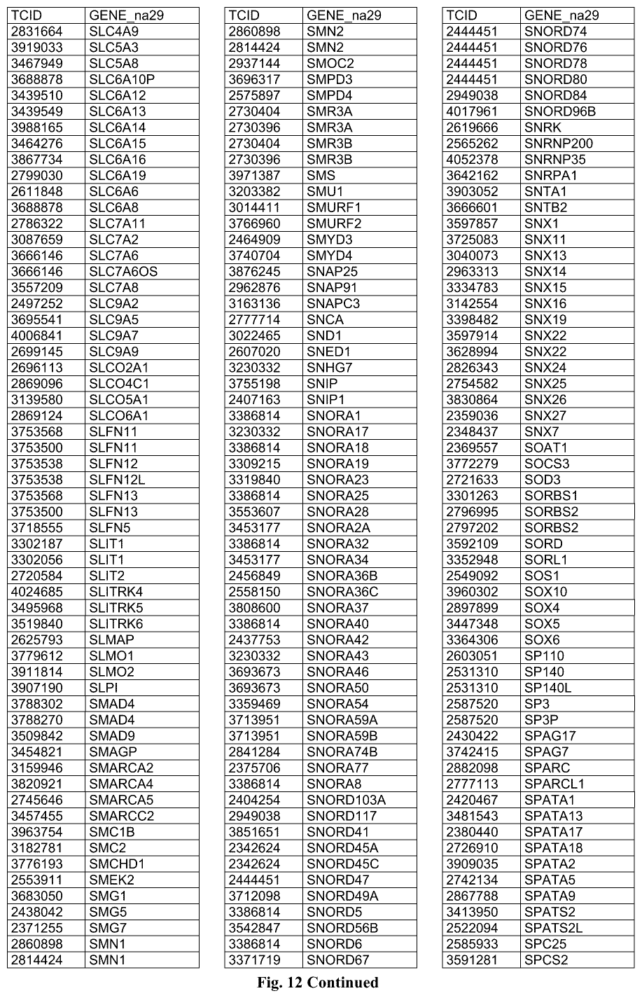

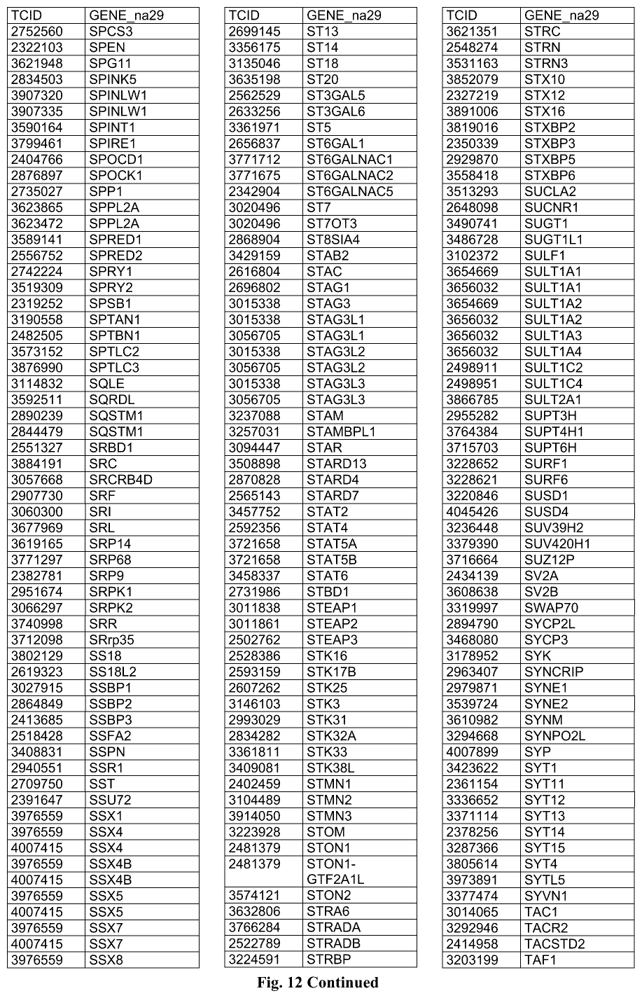

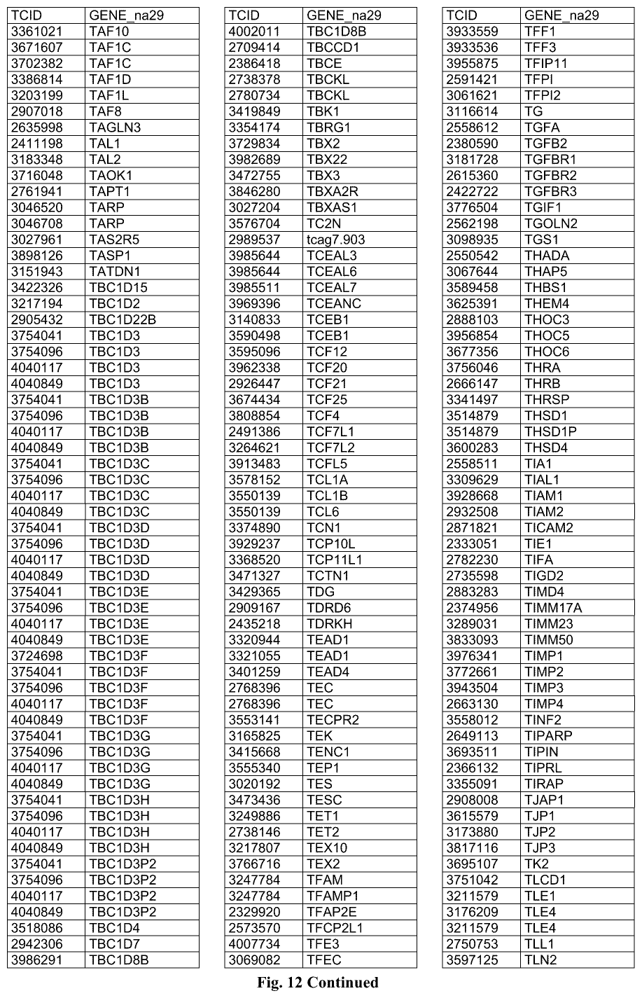

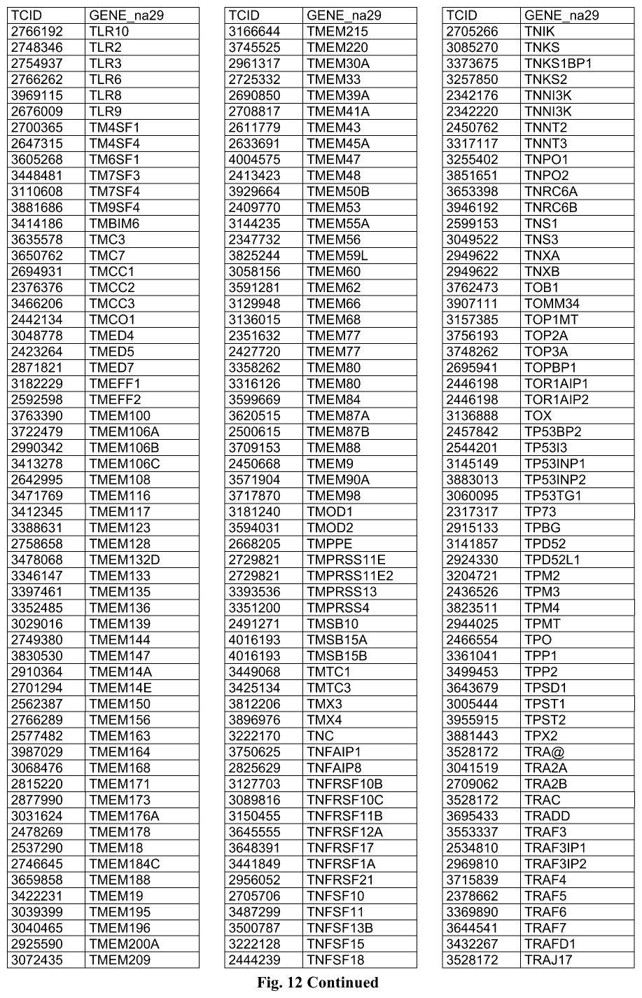

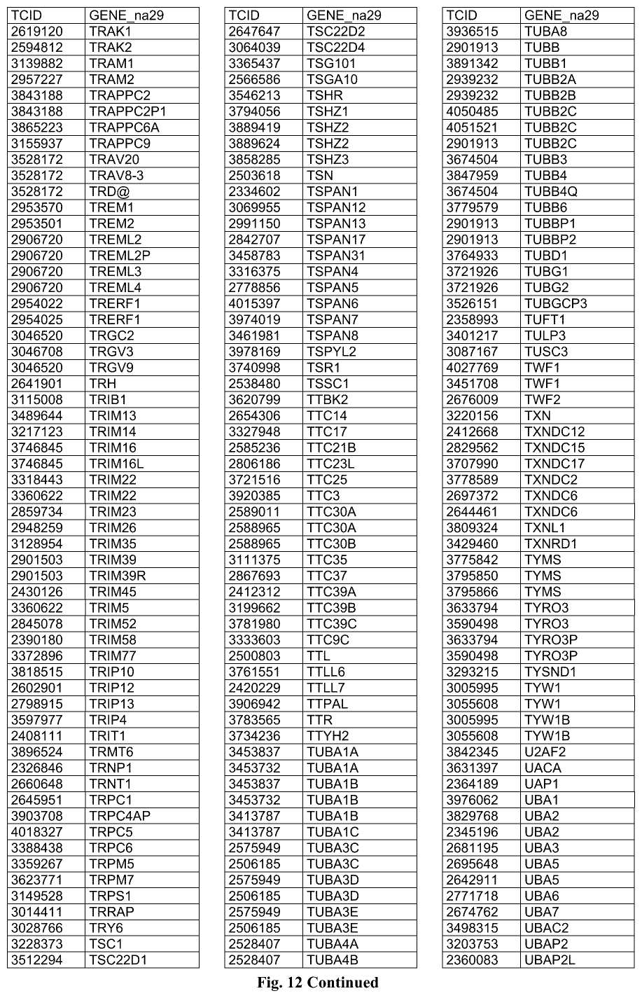

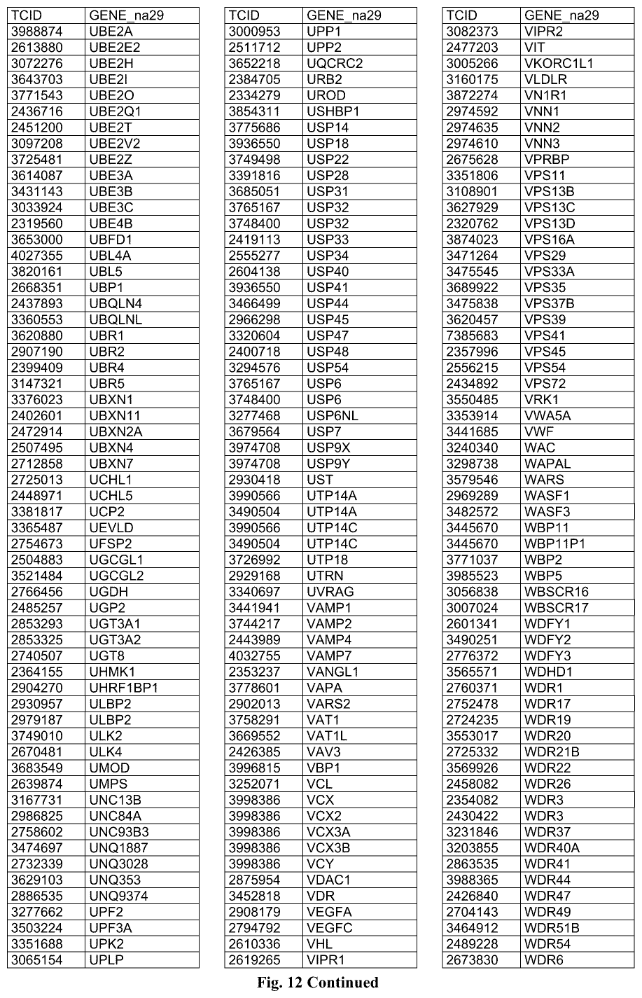

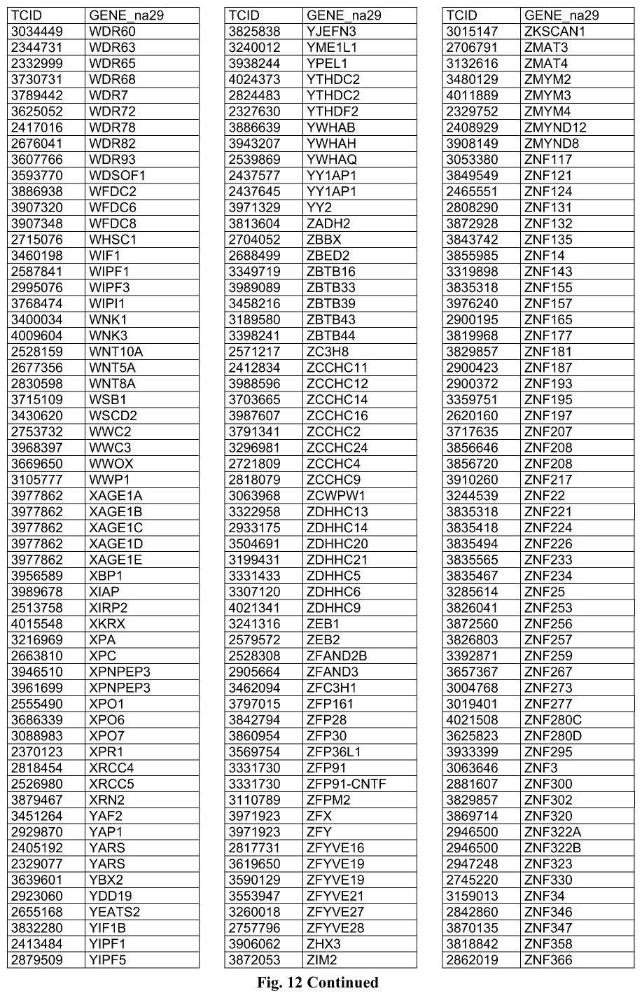

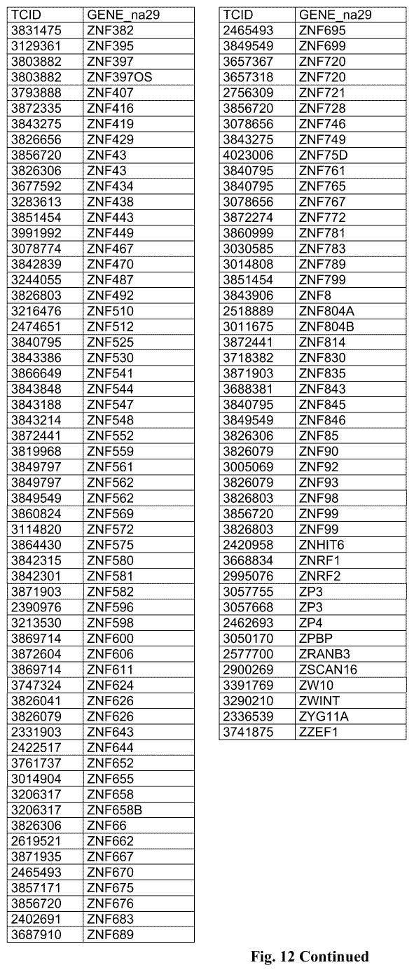

In some cases, the methods disclosed herein further comprise processing the gene expression products using an a curated panel of sequence associated with variants and/or fusions and which includes well validated variants and variants whose clinical significance is emerging (such as, for example the Xpression Atlas to provide further genomic information on samples identified as being suspicious for malignancy, or malignant, the method comprising identifying any one of the genetic aberrations disclosed in in one or more genes listed in FIG. 12 in the sample to indicate (i) risk of malignancy, (ii) a histological subtype, and (iii) prognosis associated with each of the genetic aberration identified in the sample (FIG. 9). In some examples, this may include identifying one or more genes, genetic aberrations of the one or more genes, or other genomic information disclosed in, for example, U.S. Pat. No. 8,541,170 and U.S. Patent Publication No. 2018/0016642, each of which is entirely incorporated herein by reference. Genetic aberrations may be any one or more of the DNA variants in one or more genes listed in FIG. 12. Genetic aberrations may be any one or more of the RNA fusions in one or more genes listed in FIG. 12. FIG. 10 is an example of an Xpression Atlas result that may be provided to the patient in conjunction with the GSC results on their samples to provide further genomic information comprising genetic aberrations identified in the samples and to indicate (i) risk of malignancy, (ii) a histological subtype, and (iii) prognosis associated with each of the genetic aberration identified in the sample. FIG. 8 illustrates the analytical performance of the 761 DNA variant panel and the 130 RNA fusion panel of Xpression Atlas.

The genetic aberrations may be validated or may have emerging clinical significance. The risk of malignancy may characterize one or more genetic aberrations as (1) highly associated with malignant nodules, (2) associated with both benign and malignant nodules, or (3) as having insufficient published evidence to characterize such risk. One or more genetic aberrations in one or more genes listed in FIG. 12 may be specific for cancer (e.g., malignancy). One or more genetic aberrations in one or more genes listed in FIG. 12 may occur in both benign and malignant samples.

The methods disclosed herein provide identifying one or more genetic aberrations in a sample that are indicative of a histological subtype. Histological subtypes may include classical parathyroid cancer (cPTC), infiltrative follicular variant of papillary thyroid carcinoma (infiltrative FVPTC), noninvasive encapsulated FVPTC (EFVPTC), Follicular thyroid carcinoma (FTC), and/or follicular adenomas (FA).

The methods disclosed herein comprise identifying one or more genetic aberrations in a sample to indicate prognosis associated with the genetic aberration. Prognostic information may comprise TNM stage and American Thyroid Association (ATA) risk. The TNM Staging System is based on the extent of the tumor (T), the extent of spread to the lymph nodes (N), and the presence of metastasis (M). The T category describes the original (primary) tumor. The TNM stage may comprise stages 1-4. ATA risk of recurrence staging system may comprises risk categories 1-3 which may correspond to low, intermediate, or high risk categories. The 761 nucleotide variant panel may have a PPA rate of at least 70%, 75%, 80%, 85%, 86%, 87%, 88%, 89%, 90%, 91%, 92%, 93%, 94%, 95%, 96%, 97%, 98%, 99%, or more. The 130 fusion panel may have a PPA rate of at least 70%, 75%, 80%, 85%, 86%, 87%, 88%, 89%, 90%, 91%, 92%, 93%, 94%, 95%, 96%, 97%, 98%, 99%, or more. Identification of one or more genetic aberrations may increase the risk of malignancy reported by one or more classifiers as used in the methods disclosed herein. Identification of one or more genetic aberrations may not increase the risk of malignancy reported by one or more classifiers as used in the methods disclosed herein. A reported risk of malignancy generated by one or more classifiers of the present disclosure may not be reduced in some cases where no genetic aberrations in one or more genes listed in FIG. 12 are identified.

### Samples

A sample obtained from a subject can comprise tissue, cells, cell fragments, cell organelles, nucleic acids, genes, gene fragments, expression products, gene expression products, gene expression product fragments or any combination thereof. A sample can be heterogeneous or homogenous. A sample can comprise blood, urine, cerebrospinal fluid, seminal fluid, saliva, sputum, stool, lymph fluid, tissue, or any combination thereof. A sample can be a tissue-specific sample such as a sample obtained from a thyroid, skin, heart, lung, kidney, breast, pancreas, liver, muscle, smooth muscle, bladder, gall bladder, colon, intestine, brain, esophagus, or prostate.

A sample of the present disclosure can be obtained by various methods, such as, for example, fine needle aspiration (FNA), core needle biopsy, vacuum assisted biopsy, incisional biopsy, excisional biopsy, punch biopsy, shave biopsy, skin biopsy, or any combination thereof.

FNA, also referred to as fine needle aspirate biopsy (FNAB), or needle aspirate biopsy (NAB), is a method of obtaining a small amount of tissue from a subject. FNA can be less invasive than a tissue biopsy, which may require surgery and hospitalization of the subject to obtain the tissue biopsy. The needle of a FNA method can be inserted into a tissue mass of a subject to obtain an amount of sample for further analysis. In some cases, two needles can be inserted into the tissue mass. The FNA sample obtained from the tissue mass may be acquired by one or more passages of the needle across the tissue mass. In some cases, the FNA sample can comprise less than about 6×106, 5×106, 4×106, 3×106, 2×106, 1×106 cells or less. The needle can be guided to the tissue mass by ultrasound or other imaging device. The needle can be hollow to permit recovery of the FNA sample through the needle by aspiration or vacuum or other suction techniques.

Samples obtained using methods disclosed herein, such as an FNA sample, may comprise a small sample volume. A sample volume may be less than about 500 microliters (uL), 400 uL, 300 uL, 200 uL, 100 uL, 75 uL, 50 uL, 25 uL, 20 uL, 15 uL, 10 uL, 5 uL, 1 uL, 0.5 uL, 0.1 uL, 0.01 uL or less. The sample volume may be less than about 1 uL. The sample volume may be less than about 5 uL. The sample volume may be less than about 10 uL. The sample volume may be less than about 20 uL. The sample volume may be between about 1 uL and about 10 uL. The sample volume may be between about 10 uL and about 25 uL.

Samples obtained using methods disclosed herein, such as an FNA sample, may comprise small sample weights. The sample weight, such as a tissue weight, may be less than about 100 milligrams (mg), 75 mg, 50 mg, 25 mg, 20 mg, 15 mg, 10 mg, 9 mg, 8 mg, 7 mg, 6 mg, 5 mg, 4 mg, 3 mg, 2 mg, 1 mg, 0.5 mg, 0.1 mg or less. The sample weight may be less than about 20 mg. The sample weight may be less than about 10 mg. The sample weight may be less than about 5 mg. The sample weight may be between about 5 mg and about 20 mg. The sample weight may be between about 1 mg and about 5 ng.

Samples obtained using methods disclosed herein, such as FNA, may comprise small numbers of cells. The number of cells of a single sample may be less than about 10×106, 5.5×106, 5×106, 4.5×106, 4×106, 3.5×106, 3×106, 2.5×106, 2×106, 1.5×106, 1×106, 0.5×106, 0.2×106, 0.1×106 cells or less. The number of cells of a single sample may be less than about 5×106 cells. The number of cells of a single sample may be less than about 4×106 cells. The number of cells of a single sample may be less than about 3×106 cells. The number of cells of a single sample may be less than about 2×106 cells. The number of cells of a single sample may be between about 1×106 and about 5×106 cells. The number of cells of a single sample may be between about 1×106 and about 10×106 cells.

Samples obtained using methods disclosed herein, such as FNA, may comprise small amounts of deoxyribonucleic acid (DNA) or ribonucleic acid (RNA). The amount of DNA or RNA in an individual sample may be less than about 500 nanograms (ng), 400 ng, 300 ng, 200 ng, 100 ng, 75 ng, 50 ng, 45 ng, 40 ng, 35 ng, 30 ng, 25 ng, 20 ng, 15 ng, 10 ng, 5 ng, 1 ng, 0.5 ng, 0.1 ng, or less. The amount of DNA or RNA may be less than about 40 ng. The amount of DNA or RNA may be less than about 25 ng. The amount of DNA or RNA may be less than about 15 ng. The amount of DNA or RNA may be between about 1 ng and about 25 ng. The amount of DNA or RNA may be between about 5 ng and about 50 ng.

RNA yield or RNA amount of a sample can be measured in nanogram to microgram amounts. An example of an apparatus that can be used to measure nucleic acid yield in the laboratory is a NANODROP® spectrophotometer, QUBIT® fluorometer, or QUANTUS™ fluorometer. The accuracy of a NANODROP® measurement may decrease significantly with very low RNA concentration. Quality of data obtained from the methods described herein can be dependent on RNA quantity. Meaningful gene expression or sequence variant data or others can be generated from samples having a low or un-measurable RNA concentration as measured by NANODROP®. In some cases, gene expression or sequence variant data or others can be generated from a sample having an unmeasurable RNA concentration.

The methods as described herein can be performed using samples with low quantity or quality of polynucleotides, such as DNA or RNA. A sample with low quantity or quality of RNA can be for example a degraded or partially degraded tissue sample. A sample with low quantity or quality of RNA may be a fine needle aspirate (FNA) sample. The RNA quality of a sample can be measured by a calculated RNA Integrity Number (RIN) value. The RIN value is an algorithm for assigning integrity values to RNA measurements. The algorithm can assign a 1 to 10 RIN value, where an RIN value of 10 can be completely intact RNA. A sample as described herein that comprises RNA can have an RIN value of about 9.0, 8.0, 7.0, 6.0, 5.0, 4.0, 3.0, 2.0, 1.0 or less. In some cases, a sample comprising RNA can have an RIN value equal or less than about 8.0. In some cases, a sample comprising RNA can have an RIN value equal or less than about 6.0. In some cases, a sample comprising RNA can have an RIN value equal or less than about 4.0. In some cases, a sample can have an RIN value of less than about 2.0.

A sample, such as an FNA sample, may be obtained from a subject by another individual or entity, such as a healthcare (or medical) professional or robot. A medical professional can include a physician, nurse, medical technician or other. In some cases, a physician may be a specialist, such as an oncologist, surgeon, or endocrinologist. A medical technician may be a specialist, such as a cytologist, phlebotomist, radiologist, pulmonologist or others. A medical professional may obtain a sample from a subject for testing or refer the subject to a testing center or laboratory for the submission of the sample. The medical professional may indicate to the testing center or laboratory the appropriate test or assay to perform on the sample, such as methods of the present disclosure including determining gene sequence data, gene expression levels, sequence variant data, or any combination thereof.

In some cases, a medical professional need not be involved in the initial diagnosis of a disease or the initial sample acquisition. An individual, such as the subject, may alternatively obtain a sample through the use of an over the counter kit. The kit may contain collection unit or device for obtaining the sample as described herein, a storage unit for storing the sample ahead of sample analysis, and instructions for use of the kit.

A sample can be obtained a) pre-operatively, b) post-operatively, c) after a cancer diagnosis, d) during routine screening following remission or cure of disease, e) when a subject is suspected of having a disease, f) during a routine office visit or clinical screen, g) following the request of a medical professional, or any combination thereof. Multiple samples at separate times can be obtained from the same subject, such as before treatment for a disease commences and after treatment ends, such as monitoring a subject over a time course. Multiple samples can be obtained from a subject at separate times to monitor the absence or presence of disease progression, regression, or remission in the subject.

### Cytological Analysis

The methods as described herein may include cytological analysis of samples. Examples of cytological analysis include cell staining techniques and/or microscope examination performed by any number of methods and suitable reagents including but not limited to: eosin-azure (EA) stains, hematoxylin stains, CYTO-STAIN™, papanicolaou stain, eosin, nissl stain, toluidine blue, silver stain, azocarmine stain, neutral red, or janus green. More than one stain can be used in combination with other stains. In some cases, cells are not stained at all. Cells can be fixed and/or permeabilized with for example methanol, ethanol, glutaraldehyde or formaldehyde prior to or during the staining procedure. In some cases, the cells may not be fixed. Staining procedures can also be utilized to measure the nucleic acid content of a sample, for example with ethidium bromide, hematoxylin, nissl stain or any other nucleic acid stain.

Microscope examination of cells in a sample can include smearing cells onto a slide by standard methods for cytological examination. Liquid based cytology (LBC) methods may be utilized. In some cases, LBC methods provide for an improved approach of cytology slide preparation, more homogenous samples, increased sensitivity and specificity, or improved efficiency of handling of samples, or any combination thereof. In LBC methods, samples can be transferred from the subject to a container or vial containing a LBC preparation solution such as for example CYTYC THINPREP®, SUREPATH™, or MONOPREP® or any other LBC preparation solution. Additionally, the sample may be rinsed from the collection device with LBC preparation solution into the container or vial to ensure substantially quantitative transfer of the sample. The solution containing the sample in LBC preparation solution may then be stored and/or processed by a machine or by one skilled in the art to produce a layer of cells on a glass slide. The sample may further be stained and examined under the microscope in the same way as a conventional cytological preparation.

Samples can be analyzed by immuno-histochemical staining. Immuno-histochemical staining can provide analysis of the presence, location, and distribution of specific molecules or antigens by use of antibodies in a sample (e.g. cells or tissues). Antigens can be small molecules, proteins, peptides, nucleic acids or any other molecule capable of being specifically recognized by an antibody. Samples may be analyzed by immuno-histochemical methods with or without a prior fixing and/or permeabilization step. In some cases, the antigen of interest may be detected by contacting the sample with an antibody specific for the antigen and then non-specific binding may be removed by one or more washes. The specifically bound antibodies may then be detected by an antibody detection reagent such as for example a labeled secondary antibody, or a labeled avidin/streptavidin. The antigen specific antibody can be labeled directly. Suitable labels for immunohistochemistry include but are not limited to fluorophores such as fluorescein and rhodamine, enzymes such as alkaline phosphatase and horse radish peroxidase, or radionuclides such as 32P and 125I. Gene product markers that may be detected by immuno-histochemical staining include but are not limited to Her2/Neu, Ras, Rho, EGFR, VEGFR, UbcH10, RET/PTC1, cytokeratin 20, calcitonin, GAL-3, thyroid peroxidase, or thyroglobulin.

Metrics associated with classifying a tissue sample as disclosed herein, such as sequences corresponding to mRNA transcripts, mitochondrial transcripts, and/or chromosomal loss of heterozygosity, need not be a characteristic of every cell of a sample found to comprise the tissue classification. Thus, the methods disclosed herein can be useful for classifying a tissue sample, e.g. as benign, suspicious for malignancy, or malignant for cancer, within a tissue where less than all cells within the sample exhibit a complete pattern of the gene expression levels or sequence variant data, or other data indicative of tissue classification. The gene expression levels, sequence variant data, or others may be either completely present, partially present, or absent within affected cells, as well as unaffected cells of the sample. The gene expression levels, sequence variant data, or others may be present in variable amounts within affected cells. The gene expression levels, sequence variant data, or others may be present in variable amounts within unaffected cells. In some cases, the gene expression levels of a first set of genes or the presence of one or more sequence variants in a second set of genes that correlates with a risk of malignancy occurrence can be positively detected. In some instances, positive detection can occur in at least 70%, 75%, 80%, 85%, 90%, 95%, or 100% of cells drawn from a sample. In some cases, the gene expression levels of a first set of genes or the presence of one or more sequence variants in a second set of genes can be absent. In some instances, absence of detection can occur in at least 70%, 75%, 80%, 85%, 90%, 95%, or 100% of cells of a corresponding normal or benign, non-disease sample.

Routine cytological or other assays may indicate a sample as negative (without disease), diagnostic (positive diagnosis for disease, such as cancer), ambiguous or suspicious (e.g., indeterminate) (suggestive of the presence of a disease, such as cancer), or non-diagnostic (providing inadequate information concerning the presence or absence of disease). The methods as described herein may confirm results from the routine cytological assessments or may provide an original assessment similar to a routine cytological assessment in the absence of one. The methods as described herein may classify a sample as malignant or benign, including samples found to be ambiguous, suspicious, or indeterminate. The methods may further stratify samples, such as samples known to be malignant, into low risk and medium-to-high risk groups of disease occurrence, including samples found to be ambiguous, suspicious, or indeterminate.

### Markers for Array Hybridization, Sequencing, Amplification

Suitable reagents for conducting array hybridization, nucleic acid sequencing, nucleic acid amplification or other amplification reactions include, but are not limited to, DNA polymerases, markers such as forward and reverse primers, deoxynucleotide triphosphates (dNTPs), and one or more buffers. Such reagents can include a primer that is selected for a given sequence of interest, such as the one or more genes of the first set of genes and/or second set of genes.

In such amplification reactions, one primer of a primer pair can be a forward primer complementary to a sequence of a target polynucleotide molecule (e.g. the one or more genes of the first or second sets) and one primer of a primer pair can be a reverse primer complementary to a second sequence of the target polynucleotide molecule and a target locus can reside between the first sequence and the second sequence.

The length of the forward primer and the reverse primer can depend on the sequence of the target polynucleotide (e.g. the one or more genes of the first or second sets) and the target locus. In some cases, a primer can be greater than or equal to about 5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 65, 70, 75, 80, 85, 90, 95, or about 100 nucleotides in length. As an alternative, a primer can be less than about 100, 95, 90, 85, 80, 75, 70, 65, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or about nucleotides in length. In some cases, a primer can be about 15 to about 20, about 15 to about 25, about 15 to about 30, about 15 to about 40, about 15 to about 45, about 15 to about 50, about 15 to about 55, about 15 to about 60, about 20 to about 25, about 20 to about 30, about 20 to about 35, about 20 to about 40, about 20 to about 45, about 20 to about 50, about 20 to about 55, about 20 to about 60, about 20 to about 80, or about 20 to about 100 nucleotides in length.

Primers can be designed according to known parameters for avoiding secondary structures and self-hybridization, such as primer dimer pairs. Different primer pairs can anneal and melt at about the same temperatures, for example, within 1° C., 2° C., 3° C., 4° C., 5° C., 6° C., 7° C., 8° C., 9° C. or 10° C. of another primer pair.

The target locus can be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends or 5′ ends of the plurality of template polynucleotides.

Markers (i.e., primers) for the methods described can be one or more of the same primer. In some instances, the markers can be one or more different primers such as about 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more different primers. In such examples, each primer of the one or more primers can comprise a different target or template specific region or sequence, such as the one or more genes of the first or second sets.

One or more primers can comprise a fixed panel of primers. The one or more primers can comprise at least one or more custom primers. The one or more primers can comprise at least one or more control primers. The one or more primers can comprise at least one or more housekeeping gene primers. In some instances, the one or more custom primers anneal to a target specific region or complements thereof. The one or more primers can be designed to amplify or to perform primer extension, reverse transcription, linear extension, non-exponential amplification, exponential amplification, PCR, or any other amplification method of one or more target or template polynucleotides.

Primers can incorporate additional features that allow for the detection or immobilization of the primer but do not alter a basic property of the primer (e.g., acting as a point of initiation of DNA synthesis). For example, primers can comprise a nucleic acid sequence at the 5′ end which does not hybridize to a target nucleic acid, but which facilitates cloning or further amplification, or sequencing of an amplified product. For example, the sequence can comprise a primer binding site, such as a PCR priming sequence, a sample barcode sequence, or a universal primer binding site or others.

A universal primer binding site or sequence can attach a universal primer to a polynucleotide and/or amplicon. Universal primers can include −47F (M13F), alfaMF, AOX3′, AOX5′, BGHr, CMV-30, CMV-50, CVMf, LACrmt, lamgda gt10F, lambda gt 10R, lambda gt11F, lambda gt11R, M13 rev, M13Forward(−20), M13Reverse, male, p10SEQPpQE, pA-120, pet4, pGAP Forward, pGLRVpr3, pGLpr2R, pKLAC14, pQEFS, pQERS, pucU1, pucU2, reversA, seqIREStam, seqIRESzpet, segori, seqPCR, seqpIRES−, seqpIRES+, seqpSecTag, seqpSecTag+, segretro+PSI, SP6, T3-prom, T7-prom, and T7-termInv. As used herein, attach can refer to both or either covalent interactions and noncovalent interactions. Attachment of the universal primer to the universal primer binding site may be used for amplification, detection, and/or sequencing of the polynucleotide and/or amplicon.

### Trained Algorithm

The trained algorithm of the present disclosure can be trained using a set of samples, such as a sample cohort. The sample cohort can comprise about 5, 10, 20, 30, 40, 50, 60, 70, 80, 90, 100, 150, 200, 250, 300, 350, 400, 450, 500, 600, 700, 800, 900, 1000, 2000, 3000, 4000, 5000 or more independent samples. The sample cohort can comprise about 100 independent samples. The sample cohort can comprise about 200 independent samples. The sample cohort can comprise between about 100 and about 700 independent samples. The independent samples can be from subjects having been diagnosed with a disease, such as cancer, from healthy subjects, or any combination thereof.

The sample cohort can comprise samples from about 5, 10, 20, 30, 40, 50, 60, 70, 80, 90, 100, 150, 200, 250, 300, 350, 400, 450, 500, 600, 700, 800, 900, 1000 or more different individuals. The sample cohort can comprise samples from about 100 different individuals. The sample cohort can comprise samples from about 200 different individuals. The different individuals can be individuals having been diagnosed with a disease, such as cancer, health individuals, or any combination thereof.

The sample cohort can comprise samples obtained from individuals living in at least 1, 2, 3, 4, 5, 6, 67, 8, 9, 10, 15, 20, 25, 30, 35, 40, 45, 50, 55, 60, 65, 70, 75, or 80 different geographical locations (e.g., sites spread out across a nation, such as the United States, across a continent, or across the world). Geographical locations include, but are not limited to, test centers, medical facilities, medical offices, post office addresses, cities, counties, states, nations, or continents. In some cases, a classifier that is trained using sample cohorts from the United States may need to be re-trained for use on sample cohorts from other geographical regions (e.g., India, Asia, Europe, Africa, etc.).

The trained algorithm may comprise one or more classifiers selected from the group consisting of a parathyroid classifier, a medullary thyroid cancer (MTC) classifier, a variant detection classifier, a fusion transcript detection classifier, an ensemble classifier, a follicular content index, and one or more Hurthle classifiers (e.g., a Hurthle cell index and/or a Hurthle neoplasm index). The ensemble classifier may be integrated with one or more index selected from the group consisting of a follicular content index, a Hurthle cell index, and a Hurthle neoplasm index. A parathyroid classifier may identify a presence or an absence of a parathyroid tissue in the tissue sample. A medullary thyroid cancer (MTC) classifier may identify a presence or an absence of a medullary thyroid cancer (MTC) in the tissue sample. A variant detection classifier may identify a presence or an absence of a BRAF mutation (such as BRAF V600E) in the tissue sample. A fusion transcript detection classifier may identify a presence or an absence of a RET/PTC gene fusion (such as RET/PTC1 and/or RET/PTC3 gene fusion) in the tissue sample. A follicular content index may identify follicular content in the tissue sample. A classifier may identify one or more TRK gene fusions and one or more RET alterations (e.g., a RET gene fusion).

The ensemble classifier may comprise 10,000 or more genes with a set of 1000 or more core genes. The 10,000 or more genes may improve the ensemble classifier stability against variability. The core genes may drive the prediction behavior of the ensemble model. The ensemble classifier may comprise or consist of 12 independent classifiers. The 12 independent classifiers may comprise or consist of 6 elastic net logistic regression models and 6 support vector machine models. The 6 elastic net logistic regression models may each differ from one another according to the gene sets disclosed in Table 2. The 6 support vector machine models may each differ from one another according to the gene sets disclosed in Table 2. The ensemble classifier may analyze the sequence information of expression gene products corresponding to about 10,000 genes. The ensemble classifier may analyze the sequence information of expression gene products corresponding to at least 500 genes of Table 3. The ensemble classifier may analyze the sequence information of expression gene products corresponding to at least 600 genes of Table 3. The ensemble classifier may analyze the sequence information of expression gene products corresponding to at least 700 genes of Table 3. The ensemble classifier may analyze the sequence information of expression gene products corresponding to at least 800 genes of Table 3. The ensemble classifier may analyze the sequence information of expression gene products corresponding to at least 900 genes of Table 3. The ensemble classifier may analyze the sequence information of expression gene products corresponding to at least 1000 genes of Table 3. The ensemble classifier may analyze the sequence information of expression gene products corresponding to at least 1100 genes of Table 3. The ensemble classifier may analyze the sequence information of expression gene products corresponding to at least 1200 genes of Table 3.

In some embodiments, the specificity of the present method is at least 60%, 65%, 70%, 75%, 80%, 85%, 86%, 87%, 88%, 89%, 90%, 91%, 92%, 93%, 94%, 95%, 96%, 97%, 98%, 99%, or more.

In some embodiments, the sensitivity of the present method is at least 70%, 75%, 80%, 85%, 86%, 87%, 88%, 89%, 90%, 91%, 92%, 93%, 94%, 95%, 96%, 97%, 98%, 99%, or more.

In some embodiments, the specificity is greater than or equal to 60%. The negative predictive value (NPV) is greater than or equal to 95%. In some embodiments, the NPV is at least 95%, 95.5%, 96%, 96.5%, 97%, 97.5%, 98%, 98.5%, 99%, 99.5% or more.

Sensitivity typically refers to TP/(TP+FN), where TP is true positive and FN is false negative. Number of Continued Indeterminate results divided by the total number of malignant results based on adjudicated histopathology diagnosis. Specificity typically refers to TN/(TN+FP), where TN is true negative and FP is false positive. The number of actual benign results is divided by the total number of benign results based on adjudicated histopathology diagnosis. Positive Predictive Value (PPV) may be determined by: TP/(TP+FP). Negative Predictive Value (NPV) may be determined by TN/(TN+FN).

A biological sample may be identified as cancerous with an accuracy of greater than 75%, 80%, 85%, 90%, 95%, 99% or more. In some embodiments, the biological sample is identified as cancerous with a sensitivity of greater than 90%. In some embodiments, the biological sample is identified as cancerous with a specificity of greater than 60%. In some embodiments, the biological sample is identified as cancerous or benign with a sensitivity of greater than 90% and a specificity of greater than 60%. In some embodiments, the accuracy is calculated using a trained algorithm.

Results of the expression analysis of the subject methods may provide a statistical confidence level that a given diagnosis is correct. In some embodiments, such statistical confidence level is above 85%, 90%, 91%, 92%, 93%, 94%, 95%, 96%, 97%, 98%, 99% or 99.5%.

A trained algorithm may produce a unique output each time it is run. For example, using a different sample or plurality of samples with the same classifier can produce a unique output each time the classifier is run. Using the same sample or plurality of samples with the same classifier can produce a unique output each time the classifier is run. Using the same samples to train a classifier more than one time, may result in unique outputs each time the classifier is run.

Characteristics of a sample (e.g., sequence information corresponding to mRNA expression, mitochondrial transcripts, genetic variants and/or fusion transcripts) can be analyzed using an algorithm that comprises one or more classifiers and which is trained using one or more an annotated reference sets. The identification can be performed by the classifier. More than one characteristic of a sample can be combined to generate classification of tissue sample. For example, sequence information corresponding to mRNA expression and mitochondrial transcripts can be combined and a classification can be generated from the combined data. The combining can be performed by the classifier. In another example, sequences obtained from a sample can be compared to a reference set to determine the presence of one or more sequence variants in a sample. In some cases, gene expression levels of one or more genes from a sample can be processed relative to expression levels of a reference set of genes that are used to train one or more classifiers to determine the presence of differential gene expression of one or more genes. A reference set can comprise one or more housekeeping genes. The reference set can comprise known sequence variants or expression levels of genes known to be associated with a particular disease or known to be associated with a non-disease state.

Classifiers of a trained algorithm can perform processing, combining, statistical evaluation, or further analysis of results, or any combination thereof. Separate reference sets may be provided for different features. For example, sequence variant data may be processed relative to a sequence variant data reference set. A gene expression level data may be processed relative to a gene expression level reference set. In some cases, multiple feature spaces may be processed with respect to the same reference set.

In some cases, sequence variants of a particular gene may or may not affect the gene expression level of that same gene. A sequence variant of a particular gene may affect the gene expression level of one or more different genes that may be located adjacent to and distal from the particular gene with the sequence variant. The presence of one or more sequence variants can have downstream effects on one or more genes. A sequence variant of a particular gene may perturb one or more signaling pathways, may cause ribonucleic acid (RNA) transcriptional regulation changes, may cause amplification of deoxyribonucleic acid (DNA), may cause multiple transcript copies to be produced, may cause excessive protein to be produced, may cause single base pairs, multi-base pairs, partial genes or one or more genes to be removed from the sequence.

Data from the methods described, such as gene expression levels or sequence variant data can be further analyzed using feature selection techniques such as filters which can assess the relevance of specific features by looking at the intrinsic properties of the data, wrappers which embed the model hypothesis within a feature subset search, or embedded protocols in which the search for an optimal set of features is built into a classifier algorithm.

Filters useful in the methods of the present disclosure can include, for example, (1) parametric methods such as the use of two sample t-tests, analysis of variance (ANOVA) analyses, Bayesian frameworks, or Gamma distribution models (2) model free methods such as the use of Wilcoxon rank sum tests, between-within class sum of squares tests, rank products methods, random permutation methods, or threshold number of misclassification (TNoM) which involves setting a threshold point for fold-change differences in expression between two datasets and then detecting the threshold point in each gene that minimizes the number of mis-classifications or (3) multivariate methods such as bivariate methods, correlation based feature selection methods (CFS), minimum redundancy maximum relevance methods (MRMR), Markov blanket filter methods, and uncorrelated shrunken centroid methods. Wrappers useful in the methods of the present disclosure can include sequential search methods, genetic algorithms, or estimation of distribution algorithms. Embedded protocols can include random forest algorithms, weight vector of support vector machine algorithms, or weights of logistic regression algorithms.

Statistical evaluation of the results obtained from the methods described herein can provide a quantitative value or values indicative of one or more of the following: the classification of the tissue sample; the likelihood of diagnostic accuracy; the likelihood of disease, such as cancer; the likelihood of a particular disease, such as a tissue-specific cancer, for example, thyroid cancer; and the likelihood of the success of a particular therapeutic intervention. Thus a medical professional, who may not be trained in genetics or molecular biology, need not understand gene expression level or sequence variant data results. Rather, data can be presented directly to the medical professional in its most useful form to guide care or treatment of the subject. Statistical evaluation, combination of separate data results, and reporting useful results can be performed by the trained algorithm. Statistical evaluation of results can be performed using a number of methods including, but not limited to: the students T test, the two sided T test, pearson rank sum analysis, hidden markov model analysis, analysis of q-q plots, principal component analysis, one way analysis of variance (ANOVA), two way ANOVA, and the like. Statistical evaluation can be performed by the trained algorithm.

### Diseases

A disease, as disclosed herein, can include thyroid cancer. Thyroid cancer can include any subtype of thyroid cancer, including but not limited to, any malignancy of the thyroid gland such as papillary thyroid cancer (PTC), follicular thyroid cancer (FTC), follicular variant of papillary thyroid carcinoma (FVPTC), medullary thyroid carcinoma (MTC), follicular carcinoma (FC), Hurthle cell carcinoma (HC), and/or anaplastic thyroid cancer (ATC). In some cases, the thyroid cancer can be differentiated. In some cases, the thyroid cancer can be undifferentiated.

A thyroid tissue sample can be classified using the methods of the present disclosure as comprising one or more benign or malignant tissue types (e.g. a cancer subtype), including but not limited to follicular adenoma (FA), nodular hyperplasia (NHP), lymphocytic thyroiditis (LCT), and Hurthle cell adenoma (HA), follicular carcinoma (FC), papillary thyroid carcinoma (PTC), follicular variant of papillary carcinoma (FVPTC), medullary thyroid carcinoma (MTC), Hürthle cell carcinoma (HC), and anaplastic thyroid carcinoma (ATC), renal carcinoma (RCC), breast carcinoma (BCA), melanoma (MMN), B cell lymphoma (BCL), or parathyroid (PTA).

### Monitoring of Subjects or Therapeutic Interventions Via Molecular Profiling

In the methods of the present disclosure, a subject may be monitored. For example, a subject may be diagnosed with cancer. This initial diagnosis may or may not involve the use of methods disclosed herein. The subject may be prescribed a therapeutic intervention such as a thyroidectomy for a subject suspected of having thyroid cancer. The results of the therapeutic intervention may be monitored on an ongoing basis by methods disclosed herein to detect the efficacy of the therapeutic intervention. In another example, a subject may be diagnosed with a benign tumor or a precancerous lesion or nodule, and the tumor, nodule, or lesion may be monitored on an ongoing basis by methods disclosed herein to detect any changes in the state of the tumor or lesion.

Methods disclosed herein may also be used to ascertain the potential efficacy of a specific therapeutic intervention prior to administering to a subject. For example, a subject may be diagnosed with cancer. A genomic sequence classifier (GSC) classifier along with Xpression Atlas may indicate a presence of at least one variant associated with highly malignant tumors. In such cases, therapeutic intervention may be customized to the results obtained. A tumor sample may be obtained and cultured in vitro using methods known to the art.

### Computer Systems

The present disclosure provides computer systems that are programmed to implement methods of the disclosure. FIG. 11 shows a computer system 1101 that is programmed or otherwise configured to implement the trained algorithm for the genomic sequencing classifier and/or the Xpression atlas. The computer system 1101 can regulate various aspects of the methods of the present disclosure, such as, for example, nucleic acid sequencing methods, interpretation of nucleic acid sequencing data and analysis of cellular nucleic acids, such as RNA (e.g., mRNA), and characterization of samples from sequencing data. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., medical professional, or subject). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Cater-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, results of nucleic acid sequencing, analysis of nucleic acid sequencing data, characterization of nucleic acid sequencing samples, tissue characterizations, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, initiate nucleic acid sequencing, process nucleic acid sequencing data, interpret nucleic acid sequencing results, characterize nucleic acid samples, characterize samples, etc.

### EXAMPLES

### Example 1. Training and Validation Cohorts

This study describes the blinded clinical validation of a genomic sequence classifier (GSC), implemented in accordance with the methods described herein, on a prospective multicenter-derived set of patients with FNA samples whose referral to surgery and histopathological diagnosis were determined in the absence of genomic information.

The study was approved by institution-specific institutional review boards as well as by Liberty IRB (DeLand, Florida; now Chesapeake IRB) and Copernicus Group Independent Review Board (Cary, North Carolina). All patients provided written informed consent prior to participating in the study.

The following thyroid nodule FNA samples were included in the training set, with each sample set being independent from one another (Table 1):

### ENHANCE Arm 1:

A dedicated molecular sample was obtained when the cytology specimen was collected from a nodule ≥1 cm during clinical care. Arm 2 samples were all unoperated, Bethesda II, or Bethesda III/IV and GEC benign, and lacked 2015 American Thyroid Association high suspicion sonographic pattern findings. Additionally, they had clinical follow-up (mean 23 months, range 17-32) and either a repeat FNA that was cytology benign, or had no growth (<50% increase in volume or <20% increase in 2 or more dimensions) or development of high suspicion ultrasound findings after the initial FNA. Nodules were excluded from Arm 2 if repeat FNA was Bethesda V or VI, GEC suspicious, or they underwent surgery. Arm 2 nodules served as truly benign samples, recognizing that GEC benign samples were underrepresented among operated Arm 1 samples.

### ENHANCE Arm 2:

A dedicated molecular sample was obtained when the cytology specimen was collected from a nodule ≥1 cm during clinical care. Arm 2 samples were all unoperated, Bethesda II, or Bethesda III/IV and GEC benign, and lacked 2015 American Thyroid Association high suspicion sonographic pattern findings. Additionally, they had clinical follow-up (mean 23 months, range 17-32) and either a repeat FNA that was cytology benign, or had no growth (<50% increase in volume or <20% increase in 2 or more dimensions) or development of high suspicion ultrasound findings after the initial FNA. Nodules were excluded from Aim 2 if repeat FNA was Bethesda V or VI, GEC suspicious, or they underwent surgery. Arm 2 nodules served as truly benign samples, recognizing that GEC benign samples were underrepresented among operated Arm 1 samples.

### VERA-CVP (Non Cyto-I) Samples:

Samples described in the clinical validation of the Afirma GEC1 with sufficient materials remaining. Only Bethesda II, V, and VI samples with histopathology labels defined by an expert panel of pathologists were allowed in the training set. 60% of these samples were randomly chosen into the training set.

### VERA-Train:

Samples used in the training set of the Afirma GEC.1

### VERA-Extra:

Collected and associated with histopathology labels identically to VERA-CVP, but these samples were not used in the training or validation of the Afirma GEC.

### CLIA-GEC B:

Samples from the CLIA stream that are GEC Benign. These samples do not have long term follow-up or a histopathology label. Their benign GEC prediction is used as a surrogate label in algorithm training.

TABLE 1
Composition of the core ensemble model training set.

| | Bethesda | Bethesda | Bethesda | Bethesda | Bethesda | Bethesda | |
|---|---|---|---|---|---|---|---|
| Cohort | II | III | IV | V | VI | NA | Total |
| ENHANCE | 8 | 209 | 76 | 5 | 10 | 0 | 308 |
| Arm 1 | | | | | | | |
| ENHANCE | 4 | 50 | 14 | 0 | 0 | 0 | 68 |
| Arm 2 | | | | | | | |
| VERA-CVP | 23 | 0 | 0 | 33 | 29 | 0 | 85 |
| VERA-Extra | 1 | 4 | 4 | 6 | 1 | 0 | 16 |
| VERA-Train | 0 | 4 | 6 | 7 | 16 | 13 | 46 |
| CLIA-GEC B | 0 | 47 | 7 | 0 | 0 | 57 | 111 |
| Total | 36 | 314 | 107 | 51 | 56 | 70 | 634 |
| (Proportion) | (5.7%) | (49.5%) | (16.9%) | (8.0%) | (8.8%) | (11%) | |

### Example 2. Validation Cohort

Dedicated thyroid nodule FNA specimens and surgical histopathology from nodules 1 cm or larger were collected using a prospective and blinded protocol at 49 academic and community centers in the United States from patients 21 years or older. These samples, stored at −80° C., were previously used to validate the GEC. The details of their enrollment and prespecified inclusion and exclusion criteria have been reported elsewhere. Histopathology diagnoses were previously established by an expert panel of thyroid surgical histopathologists that were blinded to all clinical and molecular data. BRAF V600E DNA mutational reference status was established by testing DNA from all samples with the competitive allele-specific TaqMan polymerase chain reaction, as described below. This independent validation cohort was prespecified and divided into a primary test set comprised of all patients with Bethesda III and IV samples described in the clinical validation of the Afirma GEC with sufficient RNA remaining and a secondary test set comprised of all patients with Bethesda II, V, or VI samples described in the clinical validation of the Afirma GEC with sufficient RNA remaining and not randomly assigned to the training set, as described in Example 1 above.

### Reference Methods:

BRAF V600E status—BRAF V600E status was determined from genomic DNA using Competitive Allele Specific Taqman PCR (castPCR™, Thermo Fisher, Waltham, MA) for BRAF 1799T>A mutation, as previously described. Briefly, genomic DNA was purified with the AllPrep Micro Kit (Qiagen, Hilden, Germany) and quantified with Quanti-iT PicoGreen dsDNA Assay Kit (Thermo Fisher, Waltham, MA). Five ng of DNA was tested with wild-type and mutant assays on an ABI7900HT. Samples were labelled BRAF V600E positive if the variant allele frequency was ≥5% and wild type if the allele frequency was <5%.

Medullary Thyroid Cancer—Histopathology diagnoses, including medullary thyroid cancer, were previously established by an expert panel of thyroid histopathologists while blinded to all clinical and molecular data.

### Example 3. Blinding of the Independent Test Set

The following steps were implemented to ensure the independent test set was securely blinded throughout algorithm development and validation.

First, each step was documented in a prespecified protocol and time-stamped on execution. Each team member was assigned a single role and allowed access only to information designated for that role. A randomly generated blinded identification number was assigned to each sample in the validation set by information technology engineers who operated independently of all other teams to ensure that all other personnel were unable to link clinical and genomic data. All historic information that may potentially reveal the clinical label on the independent test set was secured in a password-protected folder prior to the start of algorithm development. Information technology engineers conducted performance testing of the validation test set independently of all other teams.

### Example 4. RNA Purification

RNA was purified with the AllPrep Micro kit (Qiagen, Hilden, Germany) as previously described. RNA was quantified using the QuantiFluor RNA System (Promega, Madison, WI). Fluorescence was read with a Tecan Infinite 200 Pro plate reader (Tecan, Mannedorf, Switzerland). RNA Integrity Number was determined with the Bioanalyzer 2100 (Agilent, Santa Clara, CA).

### Example 5. Library Preparation

Samples were randomized and plated into 96 well plates according to their random order. Each plate contained Universal Human Reference RNA (Agilent, Santa Clara, CA), a benign thyroid tissue control sample, a malignant thyroid tissue control sample, a medullary thyroid carcinoma tissue control sample and 6 FNAs that were run on every plate in the study. Additionally, 3 samples from each plate were randomly selected to be included as technical replicates.

15 ng of total RNA was transferred to a 96 well plate. The TruSeq RNA Access Library Preparation Kit (Illumina, San Diego, CA) was adapted for use on the Microlab STAR robotics platform (Hamilton, Reno, NV). During library preparation, total RNA is fragmented, reverse transcribed, end-repaired, A-tailed, and Illumina adapters with individual indexes are ligated. Following PCR and AMpure XP (Beckman Coulter, Indianapolis, IN) cleanup, library size and quantity was determined with the Fragment Analyzer (Advanced Analytical, Ankeny, IA). 250 ng of 4 libraries were combined and sequentially captured with the human exome to remove ribosomal RNA, intronic, and intergenic sequences. Following PCR and AMpure XP (Beckman Coulter, Indianapolis, IN) cleanup, library size and quantity were determined with the Bioanalyzer 2100 (Agilent, Santa Clara, CA).

### Example 6. Next-Generation Sequencing

Libraries were normalized to 2 nM, pooled to 16 samples per sequencing run, and denatured according to the manufacturer's instructions. 1% phiX library (Illumina, San Diego, CA) was spiked into each sequencing run. Denatured and diluted libraries were loaded onto NextSeq 500 machines (Illumina, San Diego, CA) and sequenced with a NextSeq v2 High Output 150 cycle kit (Illumina, San Diego, CA) for paired end 2×76 cycle sequencing. Sequencing runs were required to have >75% of bases ≥Q30 and <1% phiX error rate.

### Example 7. RNA Sequencing Pipeline, Feature Extraction, and Quality Control

RNA-seq data was used to generate gene expression counts, identify variants, detect fusion-pairs, and calculate loss of heterozygosity (LOH) statistics. Raw sequencing data (FASTQ file) was aligned to human reference genome assembly 37 (Genome Reference Consortium) using STAR RNA-seq aligner. Expression counts were obtained by HTSeq5 and normalized using DESeq26 accounting for sequencing depth and gene-wise variability. Variants were identified using GATK variant calling pipeline, and fusion-pairs detected using STAR-Fusion. A loss of heterozygosity (LOH) statistic at chromosome and genome level was developed using variants identified genome-wide. The statistic quantifies the magnitude of LOH by calculating the proportion of variants that have a variant allele frequency (VAF; fraction of reads carrying the alternative allele) away from 0.5 (<0.2 or >0.8) after pre-filtering of variants that has a VAF exactly at zero or one, or is located in cytoband regions exhibiting abnormal excess of LOH signatures across all training samples.

To exclude low quality samples from downstream analysis, quality metrics were evaluated against pre-specified acceptance metrics for total numbers of sequenced and uniquely mapped reads, the overall proportion of exonic reads among mapped, the mean per-base coverage, the uniformity of base coverage, and base duplication and mismatch rates. All these QC metrics were generated using RNA-SeQC. Any sample that failed a QC metric was reprocessed from total RNA through library preparation and sequencing if sufficient RNA was available. Only samples passing the quality criteria were used for downstream analysis.

### Example 8. Algorithm Development

Fine-needle aspiration samples (n=634) were used to build the GSC core ensemble model, as described in Example 1. The ensemble model consists of 12 independent classifiers: 6 are elastic net logistic regression models and 6 are support vector machines. The 6 models within each category differ from each other according to the gene sets used (Table 2).

TABLE 2
Feature sets used in each classifier within the final ensemble model.

| Feature set name | Description of feature set | Size |
|---|---|---|
| DE-significant | Top significant genes at FDR-adjusted p-value < 0.05 based on | 10,158 |
| | differential expression analysis using DESeq2 package | |
| HOPACH50perc | HOPACH clustering was done on top 2,000 significant genes, then | 998 |
| | within each cluster, top 50% genes were retrieved | |
| HOPACH10perc | HOPACH clustering was done on top 2,000 significant genes, then | 196 |
| | within each cluster, top 10% genes were retrieved | |
| GEC | Among the 142 genes used by Afirma GEC main classifier, 140 gene [symbol] | 140 |
| | were targeted by RNA-sequencing | |
| GEC-HOPACH50perc | Union of ‘GEC’ and ‘HOPACH50perc’ sets | 1,115 |
| GEC-HOPACH10perc | Union of ‘GEC’ and ‘HOPACH10perc’ sets | 327 |

FDR—false discovery rate [symbol]
indicates data missing or illegible when filed

To minimize overfitting and to accurately reflect classifier performance incorporating random noise, hyperparameter tuning and model selections were performed using repeated nested cross-validation. Hyperparameter tuning was performed within the inner layer of the cross-validation, and the classifier performance was summarized using the outer layer of the 5-fold cross-validation repeated 40 times. For each classifier, the decision boundary was chosen to optimize specificity, with a minimum requirement of 90% sensitivity to detect malignancy.

The locked ensemble model uses a total of 10 196 genes, among which are 1115 core genes (Table 3). These core genes drive the prediction behavior of the model, and the remaining genes improve classifier stability against assay variability.

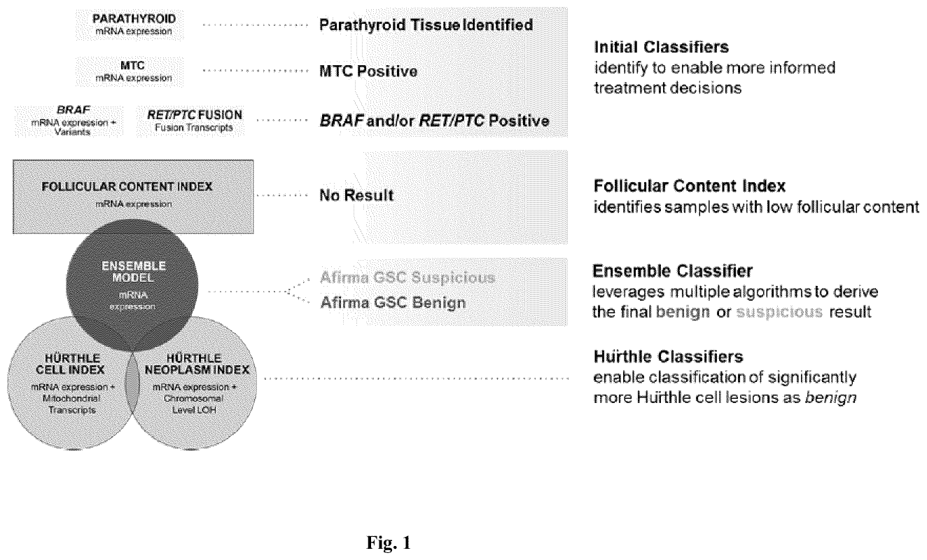

In addition to the ensemble model described above, the Afirma GSC system includes 7 other components: a parathyroid cassette, a medullary thyroid cancer (MTC) cassette, a BRAFV600E cassette, RET/PTC1 and RET/PTC3 fusion detection modules, follicular content index, Hurthle cell index, and Hurthle neoplasm index. The first 4 are upstream of the ensemble classifier, targeting specific and rare patient subgroups (FIG. 1). The last 3 (the follicular content index, Hurthle cell index, and the Hurthle neoplasm index) were developed to further improve the benign vs suspicious classification performance. They were incorporated with the ensemble classifier to form the core benign vs suspicious classifier engine.

TABLE 3
List of 1115 core genes deriving the ensemble model prediction.

| Gene_id | Gene_name | Chromosome | Start | End |
|---|---|---|---|---|
| ENSG00000121270 | ABCC11 | 16 | 48200821 | 48281479 |
| ENSG00000173208 | ABCD2 | 12 | 39943835 | 40013553 |
| ENSG00000144827 | ABHD10 | 3 | 111697857 | 111712210 |
| ENSG00000136379 | ABHD17C | 15 | 80972025 | 81047962 |
| ENSG00000166016 | ABTB2 | 11 | 34172535 | 34379555 |
| ENSG00000222482 | AC005071.1 | 7 | 99817650 | 99817743 |
| ENSG00000235978 | AC018816.3 | 3 | 4855978 | 4928977 |
| ENSG00000215067 | AC027763.2 | 17 | 6779954 | 6915668 |
| ENSG00000177076 | ACER2 | 9 | 19408925 | 19452018 |
| ENSG00000078124 | ACER3 | 11 | 76571911 | 76737841 |
| ENSG00000151726 | ACSL1 | 4 | 185676749 | 185747972 |
| ENSG00000184009 | ACTG1 | 17 | 79476997 | 79490873 |
| ENSG00000130402 | ACTN4 | 19 | 39138289 | 39222223 |
| ENSG00000115073 | ACTR1B | 2 | 98272431 | 98280570 |
| ENSG00000115170 | ACVR1 | 2 | 158592958 | 158732374 |
| ENSG00000143537 | ADAM15 | 1 | 155023042 | 155035252 |
| ENSG00000163638 | ADAMTS9 | 3 | 64501333 | 64673676 |
| ENSG00000065457 | ADAT1 | 16 | 75630879 | 75657198 |
| ENSG00000155897 | ADCY8 | 8 | 131792547 | 132054672 |
| ENSG00000156110 | ADK | 10 | 75910960 | 76469061 |
| ENSG00000163485 | ADORA1 | 1 | 203059782 | 203136533 |
| ENSG00000196526 | AFAP1 | 4 | 7760441 | 7941653 |
| ENSG00000144218 | AFF3 | 2 | 100162323 | 100759201 |
| ENSG00000038002 | AGA | 4 | 178351924 | 178363657 |
| ENSG00000188157 | AGRN | 1 | 955503 | 991496 |
| ENSG00000124942 | AHNAK | 11 | 62201016 | 62323707 |
| ENSG00000185567 | AHNAK2 | 14 | 105403581 | 105444694 |
| ENSG00000173209 | AHSA2 | 2 | 61404553 | 61418338 |
| ENSG00000163568 | AIM2 | 1 | 159032274 | 159116886 |
| ENSG00000106305 | AIMP2 | 7 | 6048876 | 6063465 |
| ENSG00000129474 | AJUBA | 14 | 23440383 | 23451851 |
| ENSG00000108599 | AKAP10 | 17 | 19807615 | 19881656 |
| ENSG00000214239 | AL591025.1 | 6 | 159047471 | 159049322 |
| ENSG00000137124 | ALDH1B1 | 9 | 38392661 | 38398658 |
| ENSG00000159063 | ALG8 | 11 | 77811982 | 77850706 |
| ENSG00000110497 | AMBRA1 | 11 | 46417964 | 46615675 |
| ENSG00000144233 | AMMECR1L | 2 | 128619204 | 128643496 |
| ENSG00000126016 | AMOT | X | 112017731 | 112084043 |
| ENSG00000131503 | ANKHD1 | 5 | 139781399 | 139929163 |
| ENSG00000144504 | ANKMY1 | 2 | 241418839 | 241508626 |
| ENSG00000167522 | ANKRD11 | 1 | 89334038 | 89556969 |
| ENSG00000174501 | ANKRD36C | 2 | 96514587 | 96657541 |
| ENSG00000135299 | ANKRD6 | 6 | 90142889 | 90343553 |
| ENSG00000163297 | ANTXR2 | 4 | 80822303 | 81046608 |
| ENSG00000135046 | ANXA1 | 9 | 75766673 | 75785309 |
| ENSG00000103723 | AP3B2 | 1 | 83328033 | 83378666 |
| ENSG00000157823 | AP3S2 | 1 | 90373831 | 90437574 |
| ENSG00000011132 | APBA3 | 1 | 3750817 | 3761697 |
| ENSG00000113108 | APBB3 | 5 | 139937853 | 139973337 |
| ENSG00000100823 | APEX1 | 1 | 20923350 | 20925927 |
| ENSG00000117362 | APH1A | 1 | 150237804 | 150241980 |
| ENSG00000084234 | APLP2 | 1 | 129939732 | 130014699 |
| ENSG00000095139 | ARCN1 | 1 | 118443105 | 118473748 |
| ENSG00000134884 | ARGLU1 | 1 | 107194021 | 107220512 |
| ENSG00000225485 | ARHGAP23 | 1 | 36584662 | 36668628 |
| ENSG00000177479 | ARIH2 | 3 | 48956254 | 49023815 |
| ENSG00000169379 | ARL13B | 3 | 93698983 | 93774512 |
| ENSG00000170632 | ARMC10 | 7 | 102715328 | 102740205 |
| ENSG00000118690 | ARMC2 | 6 | 109169619 | 109295186 |
| ENSG00000169126 | ARMC4 | 1 | 28064115 | 28287977 |
| ENSG00000102401 | ARMCX3 | X | 100877787 | 100882833 |
| ENSG00000198960 | ARMCX6 | X | 100870110 | 100872991 |
| ENSG00000241553 | ARPC4 | 3 | 9834179 | 9849410 |
| ENSG00000197070 | ARRDC1 | 9 | 140500106 | 140509812 |
| ENSG00000151693 | ASAP2 | 2 | 9346894 | 9545812 |
| ENSG00000148331 | ASB6 | 9 | 132399171 | 132404444 |
| ENSG00000112249 | ASCC3 | 6 | 100956070 | 101329248 |
| ENSG00000141505 | ASGR1 | 1 | 7076750 | 7082883 |
| ENSG00000106819 | ASPN | 9 | 95218487 | 95244788 |
| ENSG00000034533 | ASTE1 | 3 | 130732719 | 130746493 |
| ENSG00000119778 | ATAD2B | 2 | 23971534 | 24149984 |
| ENSG00000145782 | ATG12 | 5 | 115163893 | 115177555 |
| ENSG00000138363 | ATIC | 2 | 216176540 | 216214487 |
| ENSG00000068650 | ATP11A | 1 | 113344643 | 113541482 |
| ENSG00000127249 | ATP13A4 | 3 | 193119866 | 193310900 |
| ENSG00000175054 | ATR | 3 | 142168077 | 142297668 |
| ENSG00000224470 | ATXN1L | 1 | 71879894 | 71919171 |
| ENSG00000158321 | AUTS2 | 7 | 69063905 | 70258054 |
| ENSG00000179913 | B3GNT3 | 1 | 17905637 | 17923891 |
| ENSG00000175711 | B3GNTL1 | 1 | 80900031 | 81009686 |
| ENSG00000105393 | BABAM1 | 1 | 17378159 | 17392058 |
| ENSG00000186318 | BACE1 | 1 | 117156402 | 117186975 |
| ENSG00000166170 | BAG5 | 1 | 104022881 | 104029168 |
| ENSG00000140320 | BAHD1 | 1 | 40731920 | 40760441 |
| ENSG00000135298 | BAI3 | 6 | 69345259 | 70099403 |
| ENSG00000175334 | BANF1 | 1 | 65769550 | 65771620 |
| ENSG00000172530 | BANP | 1 | 87982850 | 88110924 |
| ENSG00000171552 | BCL2L1 | 2 | 30252255 | 30311792 |
| ENSG00000116128 | BCL9 | 1 | 147013182 | 147098017 |
| ENSG00000123095 | BHLHE41 | 1 | 26272959 | 26278060 |
| ENSG00000168487 | BMP1 | 8 | 22022249 | 22069839 |
| ENSG00000125378 | BMP4 | 1 | 54416454 | 54425479 |
| ENSG00000204217 | BMPR2 | 2 | 203241659 | 203432474 |
| ENSG00000163141 | BNIPL | 1 | 151009046 | 151020076 |
| ENSG00000038219 | BOD1L1 | 4 | 13570362 | 13629347 |
| ENSG00000133639 | BTG1 | 1 | 92536286 | 92539673 |
| ENSG00000186265 | BTLA | 3 | 112182815 | 112218408 |
| ENSG00000155640 | C10orf12 | 1 | 98741041 | 98745582 |
| ENSG00000158636 | C11orf30 | 1 | 76155967 | 76264069 |
| ENSG00000149179 | C11orf49 | 1 | 46958240 | 47185936 |
| ENSG00000110696 | C11orf58 | 1 | 16634679 | 16778428 |
| ENSG00000166352 | C11orf74 | 1 | 36616051 | 36694823 |
| ENSG00000173715 | C11orf80 | 1 | 66511922 | 66610987 |
| ENSG00000133935 | C14orf1 | 1 | 76116134 | 76127532 |
| ENSG00000179933 | C14orf119 | 1 | 23563974 | 23569665 |
| ENSG00000133943 | C14orf159 | 1 | 91526677 | 91691976 |
| ENSG00000168260 | C14orf183 | 1 | 50550369 | 50559361 |
| ENSG00000246223 | C14orf64 | 1 | 98391947 | 98444461 |
| ENSG00000166780 | C16orf45 | 1 | 15528152 | 15718885 |
| ENSG00000185905 | C16orf54 | 1 | 29753784 | 29757327 |
| ENSG00000205710 | C17orf107 | 1 | 4802713 | 4806227 |
| ENSG00000196544 | C17orf59 | 1 | 8091652 | 8093564 |
| ENSG00000104979 | C19orf53 | 1 | 13884982 | 13889276 |
| ENSG00000162817 | C1orf115 | 1 | 220863187 | 220872499 |
| ENSG00000182795 | C1orf116 | 1 | 207191866 | 207206101 |
| ENSG00000143612 | C1orf43 | 1 | 154179182 | 154193104 |
| ENSG00000111731 | C2CD5 | 1 | 22601517 | 22697480 |
| ENSG00000119147 | C2orf40 | 2 | 106679702 | 106694615 |
| ENSG00000118961 | C2orf43 | 2 | 20883788 | 21022882 |
| ENSG00000159239 | C2orf81 | 2 | 74641304 | 74648718 |
| ENSG00000125730 | C3 | 1 | 6677715 | 6730573 |
| ENSG00000244731 | C4A | 6 | 31949801 | 31970458 |
| ENSG00000224389 | C4B | 6 | 31982539 | 32003195 |
| ENSG00000181751 | C5orf30 | 5 | 102594403 | 102614361 |
| ENSG00000205765 | C5orf51 | 5 | 41904290 | 41921738 |
| ENSG00000203872 | C6orf163 | 6 | 88054567 | 88075181 |
| ENSG00000204387 | C6orf48 | 6 | 31802385 | 31807541 |
| ENSG00000146963 | C7orf55- | 7 | 139025105 | 139108198 |
| ENSG00000253250 | C8orf88 | 8 | 91970865 | 91997485 |
| ENSG00000136932 | C9orf156 | 9 | 100666771 | 100684852 |
| ENSG00000238227 | C9orf69 | 9 | 139006427 | 139010731 |
| ENSG00000063180 | CA11 | 1 | 49141199 | 49149569 |
| ENSG00000182985 | CADM1 | 1 | 115039938 | 115375675 |
| ENSG00000162545 | CAMK2N1 | 1 | 20808884 | 20812713 |
| ENSG00000111530 | CAND1 | 1 | 67663061 | 67713731 |
| ENSG00000014216 | CAPN1 | 1 | 64948037 | 64979477 |
| ENSG00000135387 | CAPRIN1 | 1 | 34073230 | 34122703 |
| ENSG00000110888 | CAPRIN2 | 1 | 30862486 | 30907885 |
| ENSG00000105483 | CARD8 | 1 | 48684027 | 48759203 |
| ENSG00000105974 | CAV1 | 7 | 116164839 | 116201233 |
| ENSG00000188649 | CC2D2B | 1 | 97733786 | 97792441 |
| ENSG00000169193 | CCDC126 | 7 | 23636998 | 23684327 |
| ENSG00000244607 | CCDC13 | 3 | 42734155 | 42814745 |
| ENSG00000004766 | CCDC132 | 7 | 92861653 | 92988338 |
| ENSG00000135205 | CCDC146 | 7 | 76751751 | 76958850 |
| ENSG00000153237 | CCDC148 | 2 | 159027593 | 159313265 |
| ENSG00000159588 | CCDC17 | 1 | 46085716 | 46089729 |
| ENSG00000216937 | CCDC7 | 1 | 32735068 | 32863492 |
| ENSG00000091986 | CCDC80 | 3 | 112323407 | 112368377 |
| ENSG00000149231 | CCDC82 | 1 | 96085933 | 96123087 |
| ENSG00000172724 | CCL19 | 9 | 34689564 | 34691274 |
| ENSG00000110092 | CCND1 | 1 | 69455855 | 69469242 |
| ENSG00000118971 | CCND2 | 1 | 4382938 | 4414516 |
| ENSG00000134480 | CCNH | 5 | 86687311 | 86708836 |
| ENSG00000163660 | CCNL1 | 3 | 156864297 | 156878549 |
| ENSG00000260916 | CCPG1 | 1 | 55632230 | 55700708 |
| ENSG00000115484 | CCT4 | 2 | 62095224 | 62115939 |
| ENSG00000135624 | CCT7 | 2 | 73460548 | 73480149 |
| ENSG00000177697 | CD151 | 1 | 832843 | 839831 |
| ENSG00000198087 | CD2AP | 6 | 47445525 | 47594999 |
| ENSG00000169217 | CD2BP2 | 1 | 30362087 | 30366682 |
| ENSG00000135218 | CD36 | 7 | 79998891 | 80308593 |
| ENSG00000117877 | CD3EAP | 1 | 45909467 | 45914024 |
| ENSG00000026508 | CD44 | 1 | 35160417 | 35253949 |
| ENSG00000169442 | CD52 | 1 | 26644448 | 26647014 |
| ENSG00000153283 | CD96 | 3 | 111011566 | 111384597 |
| ENSG00000105401 | CDC37 | 1 | 10501810 | 10530797 |
| ENSG00000171219 | CDC42BPG | 1 | 64590859 | 64612041 |
| ENSG00000128283 | CDC42EP1 | 2 | 37956454 | 37965412 |
| ENSG00000179604 | CDC42EP4 | 1 | 71279763 | 71308314 |
| ENSG00000140937 | CDH11 | 1 | 64977656 | 65160015 |
| ENSG00000166589 | CDH16 | 1 | 66942025 | 66952887 |
| ENSG00000124215 | CDH26 | 2 | 58533471 | 58609066 |
| ENSG00000062038 | CDH3 | 1 | 68670092 | 68756519 |
| ENSG00000179242 | CDH4 | 2 | 59827482 | 60515673 |
| ENSG00000065883 | CDK13 | 7 | 39989636 | 40136733 |
| ENSG00000136861 | CDK5RAP2 | 9 | 123151147 | 123342448 |
| ENSG00000134058 | CDK7 | 5 | 68530668 | 68573250 |
| ENSG00000100490 | CDKL1 | 1 | 50796310 | 50883179 |
| ENSG00000006837 | CDKL3 | 5 | 133541305 | 133706738 |
| ENSG00000007129 | CEACAM21 | 1 | 42055886 | 42093197 |
| ENSG00000102901 | CENPT | 1 | 67862060 | 67881714 |
| ENSG00000174799 | CEP135 | 4 | 56815037 | 56899529 |
| ENSG00000126001 | CEP250 | 2 | 34042985 | 34099804 |
| ENSG00000198707 | CEP290 | 1 | 88442793 | 88535993 |
| ENSG00000183137 | CEP57L1 | 6 | 109416313 | 109485135 |
| ENSG00000111860 | CEP85L | 6 | 118781935 | 119031238 |
| ENSG00000000971 | CFH | 1 | 196621008 | 196716634 |
| ENSG00000205403 | CFI | 4 | 110661852 | 110723335 |
| ENSG00000163320 | CGGBP1 | 3 | 88101094 | 88199035 |
| ENSG00000111642 | CHD4 | 1 | 6679249 | 6716642 |
| ENSG00000072609 | CHFR | 1 | 133398773 | 133532890 |
| ENSG00000109220 | CHIC2 | 4 | 54875956 | 54930857 |
| ENSG00000115526 | CHST10 | 2 | 101008327 | 101034118 |
| ENSG00000175040 | CHST2 | 3 | 142838173 | 142841800 |
| ENSG00000138615 | CILP | 1 | 65488337 | 65503826 |
| ENSG00000141076 | CIRH1A | 1 | 69165194 | 69265033 |
| ENSG00000125931 | CITED1 | X | 71521488 | 71527037 |
| ENSG00000273192 | CITF22-1A6.3 | 2 | 50295876 | 50298224 |
| ENSG00000104859 | CLASRP | 1 | 45542298 | 45574214 |
| ENSG00000163347 | CLDN1 | 3 | 190023490 | 190040264 |
| ENSG00000113946 | CLDN16 | 3 | 190040330 | 190129932 |
| ENSG00000189143 | CLDN4 | 7 | 73213872 | 73247014 |
| ENSG00000105270 | CLIP3 | 1 | 36505562 | 36524245 |
| ENSG00000179335 | CLK3 | 1 | 74890841 | 74932057 |
| ENSG00000188603 | CLN3 | 1 | 28477983 | 28506896 |
| ENSG00000049656 | CLPTM1L | 5 | 1317859 | 1345214 |
| ENSG00000171603 | CLSTN1 | 1 | 9789084 | 9884584 |
| ENSG00000120885 | CLU | 8 | 27454434 | 27472548 |
| ENSG00000170293 | CMTM8 | 3 | 32280171 | 32411817 |
| ENSG00000117519 | CNN3 | 1 | 95362507 | 95392834 |
| ENSG00000080802 | CNOT4 | 7 | 135046547 | 135194875 |
| ENSG00000173786 | CNP | 1 | 40118759 | 40129749 |
| ENSG00000144810 | COL8A1 | 3 | 99357319 | 99518070 |
| ENSG00000171812 | COL8A2 | 1 | 36560837 | 36590821 |
| ENSG00000169019 | COMMD8 | 4 | 47452885 | 47465736 |
| ENSG00000129083 | COPB1 | 1 | 14464986 | 14521573 |
| ENSG00000184432 | COPB2 | 3 | 139074442 | 139108574 |
| ENSG00000115520 | COQ10B | 2 | 198318147 | 198340032 |
| ENSG00000109472 | CPE | 4 | 166282346 | 166419472 |
| ENSG00000117322 | CR2 | 1 | 207627575 | 207663240 |
| ENSG00000166426 | CRABP1 | 1 | 78632666 | 78640572 |
| ENSG00000169372 | CRADD | 1 | 94071151 | 94288616 |
| ENSG00000095794 | CREM | 1 | 35415719 | 35501886 |
| ENSG00000006016 | CRLF1 | 1 | 18683030 | 18718551 |
| ENSG00000175315 | CST6 | 1 | 65779312 | 65780976 |
| ENSG00000102974 | CTCF | 1 | 67596310 | 67673086 |
| ENSG00000183248 | CTD- | 1 | 7933605 | 7939326 |
| ENSG00000044115 | CTNNA1 | 5 | 137946656 | 138270723 |
| ENSG00000066032 | CTNNA2 | 2 | 79412357 | 80875905 |
| ENSG00000119326 | CTNNAL1 | 9 | 111704851 | 111775809 |
| ENSG00000168036 | CTNNB1 | 3 | 41236328 | 41301587 |
| ENSG00000085733 | CTTN | 1 | 70244510 | 70282690 |
| ENSG00000044090 | CUL7 | 6 | 43005355 | 43021683 |
| ENSG00000108296 | CWC25 | 1 | 36956687 | 36981734 |
| ENSG00000168329 | CX3CR1 | 3 | 39304985 | 39323226 |
| ENSG00000156234 | CXCL13 | 4 | 78432907 | 78532988 |
| ENSG00000145824 | CXCL14 | 5 | 134906373 | 134914969 |
| ENSG00000103018 | CYB5B | 1 | 69458428 | 69500169 |
| ENSG00000166394 | CYB5R2 | 1 | 7686331 | 7698453 |
| ENSG00000172115 | CYCS | 7 | 25159710 | 25164980 |
| ENSG00000142973 | CYP4B1 | 1 | 47223510 | 47285085 |
| ENSG00000152207 | CYSLTR2 | 1 | 49280951 | 49283498 |
| ENSG00000108669 | CYTH1 | 1 | 76670130 | 76778379 |
| ENSG00000153071 | DAB2 | 5 | 39371780 | 39462402 |
| ENSG00000136848 | DAB2IP | 9 | 124329336 | 124547809 |
| ENSG00000115827 | DCAF17 | 2 | 172290727 | 172341562 |
| ENSG00000057019 | DCBLD2 | 3 | 98514785 | 98620533 |
| ENSG00000164935 | DCSTAMP | 8 | 105351315 | 105368917 |
| ENSG00000150401 | DCUN1D2 | 1 | 114110134 | 114145267 |
| ENSG00000178404 | DDC8 | 1 | 76866992 | 76899299 |
| ENSG00000197312 | DDI2 | 1 | 15943995 | 15995539 |
| ENSG00000089737 | DDX24 | 1 | 94517266 | 94547591 |
| ENSG00000145833 | DDX46 | 5 | 134094469 | 134190823 |
| ENSG00000118197 | DDX59 | 1 | 200593024 | 200639097 |
| ENSG00000160570 | DEDD2 | 1 | 42702750 | 42724292 |
| ENSG00000164825 | DEFB1 | 8 | 6728097 | 6735544 |
| ENSG00000105339 | DENND3 | 8 | 142127377 | 142205907 |
| ENSG00000174839 | DENND6A | 3 | 57611184 | 57678816 |
| ENSG00000023697 | DERA | 1 | 16064106 | 16190220 |
| ENSG00000183628 | DGCR6 | 2 | 18893541 | 18901751 |
| ENSG00000157680 | DGKI | 7 | 137065783 | 137531838 |
| ENSG00000172893 | DHCR7 | 1 | 71139239 | 71163914 |
| ENSG00000167536 | DHRS13 | 1 | 27224799 | 27230089 |
| ENSG00000162496 | DHRS3 | 1 | 12627939 | 12677737 |
| ENSG00000160305 | DIP2A | 2 | 47878812 | 47989926 |
| ENSG00000162595 | DIRAS3 | 1 | 68511645 | 68517314 |
| ENSG00000164741 | DLC1 | 8 | 12940870 | 13373167 |
| ENSG00000198947 | DMD | X | 31115794 | 33357558 |
| ENSG00000114841 | DNAH1 | 3 | 52350335 | 52434507 |
| ENSG00000138246 | DNAJC13 | 3 | 132136370 | 132257876 |
| ENSG00000179532 | DNHD1 | 1 | 6518490 | 6614988 |
| ENSG00000088387 | DOCK9 | 1 | 99445741 | 99738879 |
| ENSG00000125170 | DOK4 | 1 | 57505863 | 57521239 |
| ENSG00000197635 | DPP4 | 2 | 162848751 | 162931052 |
| ENSG00000130226 | DPP6 | 7 | 153584182 | 154685995 |
| ENSG00000162961 | DPY30 | 2 | 32092878 | 32264881 |
| ENSG00000113657 | DPYSL3 | 5 | 146770374 | 146889619 |
| ENSG00000175550 | DRAP1 | 1 | 65686728 | 65689032 |
| ENSG00000096696 | DSP | 6 | 7541808 | 7586950 |
| ENSG00000110042 | DTX4 | 1 | 58938903 | 58976060 |
| ENSG00000120875 | DUSP4 | 8 | 29190581 | 29208185 |
| ENSG00000138166 | DUSP5 | 1 | 112257596 | 112271302 |
| ENSG00000107404 | DVL1 | 1 | 1270656 | 1284730 |
| ENSG00000077380 | DYNC1I2 | 2 | 172543919 | 172604930 |
| ENSG00000146425 | DYNLT1 | 6 | 159057506 | 159065771 |
| ENSG00000145088 | EAF2 | 3 | 121554030 | 121605373 |
| ENSG00000255423 | EBLN2 | 3 | 73110810 | 73112488 |
| ENSG00000117298 | ECE1 | 1 | 21543740 | 21671997 |
| ENSG00000143369 | ECM1 | 1 | 150480538 | 150486265 |
| ENSG00000203734 | ECT2L | 6 | 139117063 | 139225207 |
| ENSG00000151617 | EDNRA | 4 | 148402069 | 148466106 |
| ENSG00000156508 | EEF1A1 | 6 | 74225473 | 74233520 |
| ENSG00000178852 | EFCAB13 | 1 | 45400656 | 45518678 |
| ENSG00000215529 | EFCAB8 | 2 | 31446729 | 31549006 |
| ENSG00000172638 | EFEMP2 | 1 | 65633912 | 65641063 |
| ENSG00000142634 | EFHD2 | 1 | 15736391 | 15756839 |
| ENSG00000169242 | EFNA1 | 1 | 155099936 | 155107333 |
| ENSG00000090776 | EFNB1 | X | 68048840 | 68061990 |
| ENSG00000138798 | EGF | 4 | 110834040 | 110933422 |
| ENSG00000120738 | EGR1 | 5 | 137801179 | 137805004 |
| ENSG00000115504 | EHBP1 | 2 | 62900986 | 63273622 |
| ENSG00000024422 | EHD2 | 1 | 48216600 | 48246391 |
| ENSG00000204371 | EHMT2 | 6 | 31847536 | 31865464 |
| ENSG00000084623 | EIF3I | 1 | 32687529 | 32697205 |
| ENSG00000156976 | EIF4A2 | 3 | 186500994 | 186507689 |
| ENSG00000109381 | ELF2 | 4 | 139949266 | 140098372 |
| ENSG00000163435 | ELF3 | 1 | 201977073 | 201986316 |
| ENSG00000126767 | ELK1 | X | 47494920 | 47510003 |
| ENSG00000155849 | ELMO1 | 7 | 36893961 | 37488852 |
| ENSG00000102890 | ELMO3 | 1 | 67233014 | 67237932 |
| ENSG00000213853 | EMP2 | 1 | 10622279 | 10674555 |
| ENSG00000131355 | EMR3 | 1 | 14729929 | 14800839 |
| ENSG00000149218 | ENDOD1 | 1 | 94822974 | 94865809 |
| ENSG00000167280 | ENGASE | 1 | 77071021 | 77084681 |
| ENSG00000167302 | ENTHD2 | 1 | 79202077 | 79212891 |
| ENSG00000183317 | EPHA10 | 1 | 38179552 | 38230805 |
| ENSG00000142627 | EPHA2 | 1 | 16450832 | 16482582 |
| ENSG00000116106 | EPHA4 | 2 | 222282747 | 222438922 |
| ENSG00000182580 | EPHB3 | 3 | 184279572 | 184300197 |
| ENSG00000227184 | EPPK1 | 8 | 144939497 | 144952632 |
| ENSG00000151491 | EPS8 | 1 | 15773092 | 16035263 |
| ENSG00000065361 | ERBB3 | 1 | 56473641 | 56497289 |
| ENSG00000104714 | ERICH1 | 8 | 564746 | 688106 |
| ENSG00000107566 | ERLIN1 | 1 | 101909851 | 101948091 |
| ENSG00000116285 | ERRFI1 | 1 | 8064464 | 8086368 |
| ENSG00000091831 | ESR1 | 6 | 151977826 | 152450754 |
| ENSG00000105755 | ETHE1 | 1 | 44010871 | 44031396 |
| ENSG00000143845 | ETNK2 | 1 | 204100190 | 204121307 |
| ENSG00000175832 | ETV4 | 1 | 41605212 | 41656988 |
| ENSG00000167880 | EVPL | 1 | 74000583 | 74023533 |
| ENSG00000170323 | FABP4 | 8 | 82390654 | 82395498 |
| ENSG00000103876 | FAH | 1 | 80444832 | 80479288 |
| ENSG00000183688 | FAM101B | 1 | 289769 | 295730 |
| ENSG00000136830 | FAM129B | 9 | 130267618 | 130341268 |
| ENSG00000152380 | FAM151B | 5 | 79783788 | 79838382 |
| ENSG00000146067 | FAM193B | 5 | 176946789 | 176981542 |
| ENSG00000198673 | FAM19A2 | 1 | 62102040 | 62672931 |
| ENSG00000108950 | FAM20A | 1 | 66531254 | 66597530 |
| ENSG00000205085 | FAM71F2 | 7 | 128312342 | 128326929 |
| ENSG00000126882 | FAM78A | 9 | 134133463 | 134151934 |
| ENSG00000162981 | FAM84A | 2 | 14772810 | 14790933 |
| ENSG00000171262 | FAM98B | 1 | 38746328 | 38779911 |
| ENSG00000197601 | FAR1 | 1 | 13690217 | 13753893 |
| ENSG00000146267 | FAXC | 6 | 99719045 | 99797938 |
| ENSG00000170271 | FAXDC2 | 5 | 154198051 | 154238812 |
| ENSG00000142449 | FBN3 | 1 | 8130286 | 8214730 |
| ENSG00000116661 | FBXO2 | 1 | 11708424 | 11715842 |
| ENSG00000135108 | FBXO21 | 1 | 117581146 | 117628336 |
| ENSG00000181617 | FDCSP | 4 | 71091788 | 71100969 |
| ENSG00000214814 | FER1L6 | 8 | 124864227 | 125132302 |
| ENSG00000113578 | FGF1 | 5 | 141971743 | 142077617 |
| ENSG00000138685 | FGF2 | 4 | 123747863 | 123819391 |
| ENSG00000127951 | FGL2 | 7 | 76822688 | 76829143 |
| ENSG00000125848 | FLRT3 | 2 | 14303634 | 14318262 |
| ENSG00000115414 | FN1 | 2 | 216225163 | 216300895 |
| ENSG00000115226 | FNDC4 | 2 | 27714750 | 27718112 |
| ENSG00000137166 | FOXP4 | 6 | 41514164 | 41570122 |
| ENSG00000171049 | FPR2 | 1 | 52255279 | 52273779 |
| ENSG00000150893 | FREM2 | 1 | 39261266 | 39460074 |
| ENSG00000111816 | FRK | 6 | 116252312 | 116381921 |
| ENSG00000172159 | FRMD3 | 9 | 85857905 | 86153461 |
| ENSG00000139926 | FRMD6 | 1 | 51955818 | 52197445 |
| ENSG00000075539 | FRYL | 4 | 48499378 | 48782339 |
| ENSG00000070404 | FSTL3 | 1 | 676392 | 683385 |
| ENSG00000137726 | FXYD6 | 1 | 117707693 | 117748201 |
| ENSG00000157240 | FZD1 | 7 | 90893783 | 90898123 |
| ENSG00000164930 | FZD6 | 8 | 104310661 | 104345094 |
| ENSG00000155760 | FZD7 | 2 | 202899310 | 202903160 |
| ENSG00000123689 | G0S2 | 1 | 209848765 | 209849733 |
| ENSG00000136928 | GABBR2 | 9 | 101050391 | 101471479 |
| ENSG00000145864 | GABRB2 | 5 | 160715436 | 160976050 |
| ENSG00000182256 | GABRG3 | 1 | 27216429 | 27778373 |
| ENSG00000116717 | GADD45A | 1 | 68150744 | 68154021 |
| ENSG00000197093 | GAL3ST4 | 7 | 99756867 | 99766373 |
| ENSG00000117308 | GALE | 1 | 24122089 | 24127271 |
| ENSG00000119514 | GALNT12 | 9 | 101569981 | 101612363 |
| ENSG00000109586 | GALNT7 | 4 | 174089904 | 174245118 |
| ENSG00000114480 | GBE1 | 3 | 81538850 | 81811312 |
| ENSG00000006625 | GGCT | 7 | 30536237 | 30591095 |
| ENSG00000146830 | GIGYF1 | 7 | 100277130 | 100287071 |
| ENSG00000213203 | GIMAP1 | 7 | 150413645 | 150421372 |
| ENSG00000106560 | GIMAP2 | 7 | 150382785 | 150390729 |
| ENSG00000133574 | GIMAP4 | 7 | 150264365 | 150271041 |
| ENSG00000145723 | GIN1 | 5 | 102421704 | 102455855 |
| ENSG00000139436 | GIT2 | 1 | 110367607 | 110434194 |
| ENSG00000187513 | GJA4 | 1 | 35258599 | 35261348 |
| ENSG00000188910 | GJB3 | 1 | 35246790 | 35251970 |
| ENSG00000166105 | GLB1L3 | 1 | 134144139 | 134189458 |
| ENSG00000186417 | GLDN | 1 | 51633826 | 51700210 |
| ENSG00000250571 | GLI4 | 8 | 144349603 | 144359101 |
| ENSG00000135423 | GLS2 | 1 | 56864736 | 56882198 |
| ENSG00000063169 | GLTSCR1 | 1 | 48111453 | 48206533 |
| ENSG00000168237 | GLYCTK | 3 | 52321105 | 52329272 |
| ENSG00000130755 | GMFG | 1 | 39818993 | 39833012 |
| ENSG00000204590 | GNL1 | 6 | 30509154 | 30524951 |
| ENSG00000130119 | GNL3L | X | 54556644 | 54587504 |
| ENSG00000136935 | GOLGA1 | 9 | 127640646 | 127710771 |
| ENSG00000174567 | GOLT1A | 1 | 204167288 | 204183220 |
| ENSG00000115806 | GORASP2 | 2 | 171784974 | 171823639 |
| ENSG00000120053 | GOT1 | 1 | 101156627 | 101190381 |
| ENSG00000204438 | GPANK1 | 6 | 31629006 | 31634060 |
| ENSG00000089916 | GPATCH2L | 1 | 76618259 | 76720685 |
| ENSG00000183484 | GPR132 | 1 | 105515728 | 105531782 |
| ENSG00000163328 | GPR155 | 2 | 175296966 | 175351822 |
| ENSG00000143147 | GPR161 | 1 | 168053997 | 168106821 |
| ENSG00000147138 | GPR174 | X | 78426469 | 78427726 |
| ENSG00000166073 | GPR176 | 1 | 40091233 | 40213093 |
| ENSG00000188394 | GPR21 | 9 | 125796806 | 125797975 |
| ENSG00000167191 | GPRC5B | 1 | 19868616 | 19897489 |
| ENSG00000141738 | GRB7 | 1 | 37894180 | 37903544 |
| ENSG00000158055 | GRHL3 | 1 | 24645812 | 24690972 |
| ENSG00000148180 | GSN | 9 | 123970072 | 124095121 |
| ENSG00000172986 | GXYLT2 | 3 | 72937224 | 73047289 |
| ENSG00000113088 | GZMK | 5 | 54320081 | 54330398 |
| ENSG00000214367 | HAUS3 | 4 | 2229191 | 2243891 |
| ENSG00000068024 | HDAC4 | 2 | 239969864 | 240323348 |
| ENSG00000173064 | HECTD4 | 1 | 112597992 | 112819896 |
| ENSG00000198265 | HELZ | 1 | 65066554 | 65242105 |
| ENSG00000103657 | HERC1 | 1 | 63900817 | 64126141 |
| ENSG00000135547 | HEY2 | 6 | 126068810 | 126082415 |
| ENSG00000163909 | HEYL | 1 | 40089825 | 40105617 |
| ENSG00000165102 | HGSNAT | 8 | 42995556 | 43057998 |
| ENSG00000196312 | HIATL2 | 9 | 99660348 | 99775862 |
| ENSG00000169567 | HINT1 | 5 | 130494720 | 130507428 |
| ENSG00000204632 | HLA-G | 6 | 29794744 | 29798902 |
| ENSG00000149948 | HMGA2 | 1 | 66217911 | 66360075 |
| ENSG00000189403 | HMGB1 | 1 | 31032884 | 31191734 |
| ENSG00000198830 | HMGN2 | 1 | 26798941 | 26802463 |
| ENSG00000177733 | HNRNPA0 | 5 | 137087075 | 137090039 |
| ENSG00000127483 | HP1BP3 | 1 | 21069154 | 21113816 |
| ENSG00000116983 | HPCAL4 | 1 | 40144320 | 40157361 |
| ENSG00000105707 | HPN | 1 | 35531410 | 35557475 |
| ENSG00000025423 | HSD17B6 | 1 | 57145945 | 57181574 |
| ENSG00000096384 | HSP90AB1 | 6 | 44214824 | 44221620 |
| ENSG00000113013 | HSPA9 | 5 | 137890571 | 137911133 |
| ENSG00000068001 | HYAL2 | 3 | 50355221 | 50360337 |
| ENSG00000242028 | HYPK | 1 | 44088340 | 44095241 |
| ENSG00000105376 | ICAM5 | 1 | 10400657 | 10407454 |
| ENSG00000116237 | ICMT | 1 | 6281253 | 6296032 |
| ENSG00000115738 | ID2 | 2 | 8818975 | 8824583 |
| ENSG00000188483 | IER5L | 9 | 131937835 | 131940540 |
| ENSG00000010295 | IFFO1 | 1 | 6647541 | 6665239 |
| ENSG00000114446 | IFT57 | 3 | 107879659 | 107941417 |
| ENSG00000073792 | IGF2BP2 | 3 | 185361527 | 185542844 |
| ENSG00000115461 | IGFBP5 | 2 | 217536828 | 217560248 |
| ENSG00000167779 | IGFBP6 | 1 | 53491220 | 53496129 |
| ENSG00000182700 | IGIP | 5 | 139505521 | 139508391 |
| ENSG00000147255 | IGSF1 | X | 130407480 | 130533677 |
| ENSG00000162729 | IGSF8 | 1 | 160061130 | 160068733 |
| ENSG00000104365 | IKBKB | 8 | 42128820 | 42189973 |
| ENSG00000030419 | IKZF2 | 2 | 213864429 | 214017151 |
| ENSG00000144730 | IL17RD | 3 | 57124010 | 57204334 |
| ENSG00000115602 | IL1RL1 | 2 | 102927962 | 102968497 |
| ENSG00000134352 | IL6ST | 5 | 55230923 | 55290821 |
| ENSG00000168685 | IL7R | 5 | 35852797 | 35879705 |
| ENSG00000143621 | ILF2 | 1 | 153634512 | 153643524 |
| ENSG00000178035 | IMPDH2 | 3 | 49061758 | 49066841 |
| ENSG00000163083 | INHBB | 2 | 121103719 | 121109384 |
| ENSG00000241644 | INMT | 7 | 30737601 | 30797218 |
| ENSG00000185085 | INTS5 | 1 | 62414320 | 62420774 |
| ENSG00000164941 | INTS8 | 8 | 95825539 | 95893974 |
| ENSG00000074706 | IPCEF1 | 6 | 154475631 | 154677926 |
| ENSG00000205339 | IPO7 | 1 | 9406169 | 9469673 |
| ENSG00000132321 | IQCA1 | 2 | 237232794 | 237416185 |
| ENSG00000145703 | IQGAP2 | 5 | 75699074 | 76003957 |
| ENSG00000066583 | ISOC1 | 5 | 128430444 | 128449721 |
| ENSG00000105655 | ISYNA1 | 1 | 18545198 | 18549111 |
| ENSG00000164171 | ITGA2 | 5 | 52285156 | 52390609 |
| ENSG00000005884 | ITGA3 | 1 | 48133332 | 48167845 |
| ENSG00000135424 | ITGA7 | 1 | 56078352 | 56109827 |
| ENSG00000144668 | ITGA9 | 3 | 37493606 | 37865005 |
| ENSG00000132470 | ITGB4 | 1 | 73717408 | 73753899 |
| ENSG00000105855 | ITGB8 | 7 | 20370325 | 20455377 |
| ENSG00000135916 | ITM2C | 2 | 231729354 | 231743963 |
| ENSG00000086544 | ITPKC | 1 | 41223008 | 41246765 |
| ENSG00000096433 | ITPR3 | 6 | 33588142 | 33664351 |
| ENSG00000205730 | ITPRIPL2 | 1 | 19125254 | 19132946 |
| ENSG00000077684 | JADE1 | 4 | 129730779 | 129796379 |
| ENSG00000102221 | JADE3 | X | 46771711 | 46920641 |
| ENSG00000171135 | JAGN1 | 3 | 9932238 | 9936033 |
| ENSG00000171988 | JMJD1C | 1 | 64926981 | 65225722 |
| ENSG00000130522 | JUND | 1 | 18390563 | 18392432 |
| ENSG00000197256 | KANK2 | 1 | 11274943 | 11308467 |
| ENSG00000114982 | KANSL3 | 2 | 97258907 | 97308524 |
| ENSG00000177272 | KCNA3 | 1 | 111214310 | 111217655 |
| ENSG00000151704 | KCNJ1 | 1 | 128706210 | 128737268 |
| ENSG00000124249 | KCNK15 | 2 | 43374421 | 43379675 |
| ENSG00000164626 | KCNK5 | 6 | 39156749 | 39197226 |
| ENSG00000184156 | KCNQ3 | 8 | 133133108 | 133493200 |
| ENSG00000174943 | KCTD13 | 1 | 29916333 | 29938356 |
| ENSG00000100196 | KDELR3 | 2 | 38864067 | 38879452 |
| ENSG00000004487 | KDM1A | 1 | 23345941 | 23410182 |
| ENSG00000127663 | KDM4B | 1 | 4969125 | 5153606 |
| ENSG00000117139 | KDM5B | 1 | 202696526 | 202778598 |
| ENSG00000165757 | KIAA1462 | 1 | 30301729 | 30404423 |
| ENSG00000134444 | KIAA1468 | 1 | 59854491 | 59974355 |
| ENSG00000166004 | KIAA1731 | 1 | 93394805 | 93463522 |
| ENSG00000173214 | KIAA1919 | 6 | 111580551 | 111592370 |
| ENSG00000157404 | KIT | 4 | 55524085 | 55606881 |
| ENSG00000102554 | KLF5 | 1 | 73629114 | 73651676 |
| ENSG00000162873 | KLHDC8A | 1 | 205305220 | 205326218 |
| ENSG00000129451 | KLK10 | 1 | 51515995 | 51523431 |
| ENSG00000169035 | KLK7 | 1 | 51479729 | 51487355 |
| ENSG00000139187 | KLRG1 | 1 | 9102640 | 9163356 |
| ENSG00000025800 | KPNA6 | 1 | 32573639 | 32642169 |
| ENSG00000111057 | KRT18 | 1 | 53342655 | 53346685 |
| ENSG00000171345 | KRT19 | 1 | 39679869 | 39684560 |
| ENSG00000157992 | KRTCAP3 | 2 | 27665233 | 27669348 |
| ENSG00000141068 | KSR1 | 1 | 25783670 | 25953461 |
| ENSG00000159166 | LAD1 | 1 | 201342372 | 201368736 |
| ENSG00000196878 | LAMB3 | 1 | 209788215 | 209825811 |
| ENSG00000135862 | LAMC1 | 1 | 182992595 | 183114727 |
| ENSG00000058085 | LAMC2 | 1 | 183155373 | 183214035 |
| ENSG00000068697 | LAPTM4A | 2 | 20232411 | 20251789 |
| ENSG00000107929 | LARP4B | 1 | 855484 | 977564 |
| ENSG00000135338 | LCA5 | 6 | 80194708 | 80247175 |
| ENSG00000205629 | LCMT1 | 1 | 25123050 | 25189552 |
| ENSG00000136167 | LCP1 | 1 | 46700055 | 46786006 |
| ENSG00000182195 | LDOC1 | X | 140269934 | 140271310 |
| ENSG00000225880 | LINC00115 | 1 | 761586 | 762902 |
| ENSG00000260032 | LINC00657 | 2 | 34633544 | 34638882 |
| ENSG00000163898 | LIPH | 3 | 185224050 | 185270401 |
| ENSG00000131899 | LLGL1 | 1 | 18128901 | 18148189 |
| ENSG00000168216 | LMBRD1 | 6 | 70385694 | 70507003 |
| ENSG00000160789 | LMNA | 1 | 156052364 | 156109880 |
| ENSG00000048540 | LMO3 | 1 | 16701307 | 16763528 |
| ENSG00000143013 | LMO4 | 1 | 87794151 | 87814606 |
| ENSG00000170500 | LONRF2 | 2 | 100889753 | 100939195 |
| ENSG00000167210 | LOXHD1 | 1 | 44056935 | 44236996 |
| ENSG00000186001 | LRCH3 | 3 | 197518097 | 197615307 |
| ENSG00000077454 | LRCH4 | 7 | 100169855 | 100183776 |
| ENSG00000147650 | LRP12 | 8 | 105501459 | 105601252 |
| ENSG00000168702 | LRP1B | 2 | 140988992 | 142889270 |
| ENSG00000134569 | LRP4 | 1 | 46878419 | 46940193 |
| ENSG00000214954 | LRRC69 | 8 | 92114060 | 92231464 |
| ENSG00000093167 | LRRFIP2 | 3 | 37094117 | 37225180 |
| ENSG00000105699 | LSR | 1 | 35739233 | 35758867 |
| ENSG00000119681 | LTBP2 | 1 | 74964873 | 75079306 |
| ENSG00000168056 | LTBP3 | 1 | 65306276 | 65326401 |
| ENSG00000198862 | LTN1 | 2 | 30300466 | 30365270 |
| ENSG00000176018 | LYSMD3 | 5 | 89811428 | 89825401 |
| ENSG00000183742 | MACC1 | 7 | 20174278 | 20257027 |
| ENSG00000172264 | MACROD2 | 2 | 13976015 | 16033842 |
| ENSG00000198517 | MAFK | 7 | 1570350 | 1582679 |
| ENSG00000081026 | MAGI3 | 1 | 113933371 | 114228545 |
| ENSG00000161021 | MAML1 | 5 | 179159851 | 179223512 |
| ENSG00000013619 | MAMLD1 | X | 149529689 | 149682448 |
| ENSG00000078018 | MAP2 | 2 | 210288782 | 210598842 |
| ENSG00000107968 | MAP3K8 | 1 | 30722866 | 30750762 |
| ENSG00000156711 | MAPK13 | 6 | 36095586 | 36107842 |
| ENSG00000138834 | MAPK8IP3 | 1 | 1756184 | 1820318 |
| ENSG00000075413 | MARK3 | 1 | 103851729 | 103970168 |
| ENSG00000132561 | MATN2 | 8 | 98881068 | 99048944 |
| ENSG00000015479 | MATR3 | 5 | 138609441 | 138667360 |
| ENSG00000146701 | MDH2 | 7 | 75677369 | 75696826 |
| ENSG00000110492 | MDK | 1 | 46402306 | 46405375 |
| ENSG00000111554 | MDM1 | 1 | 68666223 | 68726161 |
| ENSG00000198625 | MDM4 | 1 | 204485511 | 204542871 |
| ENSG00000124733 | MEA1 | 6 | 42979832 | 42981706 |
| ENSG00000163875 | MEAF6 | 1 | 37958176 | 37980375 |
| ENSG00000085276 | MECOM | 3 | 168801287 | 169381406 |
| ENSG00000144893 | MED12L | 3 | 150803484 | 151154860 |
| ENSG00000108510 | MED13 | 1 | 60019966 | 60142643 |
| ENSG00000102802 | MEDAG | 1 | 31480328 | 31499709 |
| ENSG00000105976 | MET | 7 | 116312444 | 116438440 |
| ENSG00000165792 | METTL17 | 1 | 21457929 | 21465189 |
| ENSG00000123427 | METTL21B | 1 | 58165275 | 58176324 |
| ENSG00000170439 | METTL7B | 1 | 56075330 | 56078395 |
| ENSG00000181588 | MEX3D | 1 | 1554668 | 1568057 |
| ENSG00000140545 | MFGE8 | 1 | 89441916 | 89456642 |
| ENSG00000174514 | MFSD4 | 1 | 205538013 | 205572046 |
| ENSG00000151690 | MFSD6 | 2 | 191273081 | 191373931 |
| ENSG00000128268 | MGAT3 | 2 | 39853349 | 39888199 |
| ENSG00000161013 | MGAT4B | 5 | 179224597 | 179233952 |
| ENSG00000008394 | MGST1 | 1 | 16500076 | 16762193 |
| ENSG00000177427 | MIEF2 | 1 | 18163848 | 18169866 |
| ENSG00000100253 | MIOX | 2 | 50925213 | 50929077 |
| ENSG00000207939 | MIR223 | X | 65238712 | 65238821 |
| ENSG00000202566 | MIR421 | X | 73438212 | 73438296 |
| ENSG00000207652 | MIR621 | 1 | 41384902 | 41384997 |
| ENSG00000207997 | MIR644A | 2 | 33054130 | 33054223 |
| ENSG00000167842 | MIS12 | 1 | 5389605 | 5394134 |
| ENSG00000196588 | MKL1 | 2 | 40806285 | 41032706 |
| ENSG00000130396 | MLLT4 | 6 | 168227602 | 168372703 |
| ENSG00000175727 | MLXIP | 1 | 122516628 | 122631894 |
| ENSG00000133131 | MORC4 | X | 106057101 | 106243474 |
| ENSG00000185787 | MORF4L1 | 1 | 79102829 | 79190475 |
| ENSG00000060762 | MPC1 | 6 | 166778407 | 166796486 |
| ENSG00000197629 | MPEG1 | 1 | 58975983 | 58980424 |
| ENSG00000103152 | MPG | 1 | 127006 | 135852 |
| ENSG00000051825 | MPHOSPH9 | 1 | 123636867 | 123728561 |
| ENSG00000130830 | MPP1 | X | 154006959 | 154049282 |
| ENSG00000066382 | MPPED2 | 1 | 30406040 | 30608419 |
| ENSG00000149573 | MPZL2 | 1 | 118124118 | 118135251 |
| ENSG00000011028 | MRC2 | 1 | 60704762 | 60770958 |
| ENSG00000173141 | MRP63 | 1 | 21750784 | 21753223 |
| ENSG00000180992 | MRPL14 | 6 | 44081194 | 44095194 |
| ENSG00000143436 | MRPL9 | 1 | 151732119 | 151736040 |
| ENSG00000102738 | MRPS31 | 1 | 41303432 | 41345309 |
| ENSG00000166928 | MS4A14 | 1 | 60146003 | 60185161 |
| ENSG00000052802 | MSMO1 | 4 | 166248775 | 166264312 |
| ENSG00000164078 | MST1R | 3 | 49924435 | 49941299 |
| ENSG00000198417 | MT1F | 1 | 56691606 | 56694610 |
| ENSG00000125144 | MT1G | 1 | 56700643 | 56701977 |
| ENSG00000205358 | MT1H | 1 | 56703726 | 56705041 |
| ENSG00000177000 | MTHFR | 1 | 11845780 | 11866977 |
| ENSG00000108389 | MTMR4 | 1 | 56566898 | 56595266 |
| ENSG00000003987 | MTMR7 | 8 | 17155539 | 17271037 |
| ENSG00000120662 | MTRF1 | 1 | 41790505 | 41837742 |
| ENSG00000132613 | MTSS1L | 1 | 70695107 | 70719969 |
| ENSG00000129422 | MTUS1 | 8 | 17501304 | 17658426 |
| ENSG00000185499 | MUC1 | 1 | 155158300 | 155162707 |
| ENSG00000204544 | MUC21 | 6 | 30951495 | 30957680 |
| ENSG00000162576 | MXRA8 | 1 | 1288069 | 1297157 |
| ENSG00000104177 | MYEF2 | 1 | 48431625 | 48470714 |
| ENSG00000133026 | MYH10 | 1 | 8377523 | 8534079 |
| ENSG00000101335 | MYL9 | 2 | 35169887 | 35178228 |
| ENSG00000196535 | MYO18A | 1 | 27400528 | 27507430 |
| ENSG00000196586 | MYO6 | 6 | 76458909 | 76629254 |
| ENSG00000172766 | NAA16 | 1 | 41885341 | 41951166 |
| ENSG00000138386 | NAB1 | 2 | 191511472 | 191557492 |
| ENSG00000166886 | NAB2 | 1 | 57482677 | 57489259 |
| ENSG00000131400 | NAPSA | 1 | 50861734 | 50869087 |
| ENSG00000185818 | NAT8L | 4 | 2061239 | 2070816 |
| ENSG00000166833 | NAV2 | 1 | 19372271 | 20143144 |
| ENSG00000114503 | NCBP2 | 3 | 196662273 | 196669468 |
| ENSG00000020129 | NCDN | 1 | 36023074 | 36032875 |
| ENSG00000178127 | NDUFV2 | 1 | 9102628 | 9134343 |
| ENSG00000188986 | NELFB | 9 | 140149625 | 140167998 |
| ENSG00000184613 | NELL2 | 1 | 44902058 | 45315631 |
| ENSG00000173848 | NET1 | 1 | 5454514 | 5500426 |
| ENSG00000050344 | NFE2L3 | 7 | 26191860 | 26226745 |
| ENSG00000147862 | NFIB | 9 | 14081842 | 14398982 |
| ENSG00000066248 | NGEF | 2 | 233743396 | 233877982 |
| ENSG00000064300 | NGFR | 1 | 47572655 | 47592379 |
| ENSG00000145912 | NHP2 | 5 | 177576461 | 177580968 |
| ENSG00000001461 | NIPAL3 | 1 | 24742284 | 24799466 |
| ENSG00000101882 | NKAP | X | 119059014 | 119077735 |
| ENSG00000169992 | NLGN2 | 1 | 7308193 | 7323179 |
| ENSG00000169251 | NMD3 | 3 | 160822484 | 160971320 |
| ENSG00000106100 | NOD1 | 7 | 30464143 | 30518400 |
| ENSG00000225921 | NOL7 | 6 | 13615559 | 13632971 |
| ENSG00000147140 | NONO | X | 70503042 | 70521018 |
| ENSG00000198929 | NOS1AP | 1 | 162039564 | 162353321 |
| ENSG00000213240 | NOTCH2NL | 1 | 145209119 | 145291972 |
| ENSG00000074181 | NOTCH3 | 1 | 15270444 | 15311792 |
| ENSG00000139910 | NOVA1 | 1 | 26912299 | 27066960 |
| ENSG00000086991 | NOX4 | 1 | 89057524 | 89322779 |
| ENSG00000119655 | NPC2 | 1 | 74942895 | 74960880 |
| ENSG00000107281 | NPDC1 | 9 | 139933922 | 139940655 |
| ENSG00000185864 | NPIPB4 | 1 | 21845890 | 21892148 |
| ENSG00000221890 | NPTXR | 2 | 39214457 | 39239987 |
| ENSG00000091129 | NRCAM | 7 | 107788068 | 108097161 |
| ENSG00000180530 | NRIP1 | 2 | 16333556 | 16437321 |
| ENSG00000241058 | NSUN6 | 1 | 18834490 | 18940551 |
| ENSG00000168268 | NT5DC2 | 3 | 52558386 | 52569070 |
| ENSG00000135318 | NT5E | 6 | 86159809 | 86205500 |
| ENSG00000140538 | NTRK3 | 1 | 88418230 | 88799999 |
| ENSG00000198585 | NUDT16 | 3 | 131100515 | 131107674 |
| ENSG00000186364 | NUDT17 | 1 | 145586115 | 145589439 |
| ENSG00000069248 | NUP133 | 1 | 229577045 | 229644103 |
| ENSG00000176046 | NUPR1 | 1 | 28548606 | 28550495 |
| ENSG00000167693 | NXN | 1 | 702553 | 883010 |
| ENSG00000145247 | OCIAD2 | 4 | 48887036 | 48908954 |
| ENSG00000197822 | OCLN | 5 | 68788119 | 68853931 |
| ENSG00000145623 | OSMR | 5 | 38845960 | 38945698 |
| ENSG00000155100 | OTUD6B | 8 | 92082424 | 92099323 |
| ENSG00000162881 | OXER1 | 2 | 42989642 | 42991401 |
| ENSG00000154814 | OXNAD1 | 3 | 16306706 | 16391806 |
| ENSG00000078589 | P2RY10 | X | 78200829 | 78217451 |
| ENSG00000181631 | P2RY13 | 3 | 151044100 | 151047336 |
| ENSG00000079462 | PAFAH1B3 | 1 | 42801185 | 42807698 |
| ENSG00000099864 | PALM | 1 | 708953 | 748329 |
| ENSG00000145730 | PAM | 5 | 102089685 | 102366809 |
| ENSG00000138964 | PARVG | 2 | 44568836 | 44615413 |
| ENSG00000115687 | PASK | 2 | 242045514 | 242089679 |
| ENSG00000229474 | PATL2 | 1 | 44957930 | 45003514 |
| ENSG00000173599 | PC | 1 | 66615704 | 66725847 |
| ENSG00000156453 | PCDH1 | 5 | 141232938 | 141258811 |
| ENSG00000189184 | PCDH18 | 4 | 138440072 | 138453648 |
| ENSG00000243232 | PCDHAC2 | 5 | 140345820 | 140391936 |
| ENSG00000240184 | PCDHGC3 | 5 | 140855580 | 140892542 |
| ENSG00000102109 | PCSK1N | X | 48689504 | 48694035 |
| ENSG00000154678 | PDE1C | 7 | 31790793 | 32338941 |
| ENSG00000138735 | PDE5A | 4 | 120415550 | 120550146 |
| ENSG00000073417 | PDE8A | 1 | 85523671 | 85682376 |
| ENSG00000160191 | PDE9A | 2 | 44073746 | 44195619 |
| ENSG00000131828 | PDHA1 | X | 19362011 | 19379823 |
| ENSG00000107438 | PDLIM1 | 1 | 96997329 | 97050781 |
| ENSG00000131435 | PDLIM4 | 5 | 131593364 | 131609147 |
| ENSG00000162734 | PEA15 | 1 | 160175127 | 160185166 |
| ENSG00000133027 | PEMT | 1 | 17408877 | 17495022 |
| ENSG00000112378 | PERP | 6 | 138409642 | 138428648 |
| ENSG00000143256 | PFDN2 | 1 | 161070346 | 161087901 |
| ENSG00000158571 | PFKFB1 | X | 54959394 | 55024967 |
| ENSG00000123836 | PFKFB2 | 1 | 207222801 | 207254369 |
| ENSG00000164219 | PGGT1B | 5 | 114546527 | 114598569 |
| ENSG00000101856 | PGRMC1 | X | 118370216 | 118378429 |
| ENSG00000116273 | PHF13 | 1 | 6673745 | 6684093 |
| ENSG00000116793 | PHTF1 | 1 | 114239453 | 114302111 |
| ENSG00000107537 | PHYH | 1 | 13319796 | 13344412 |
| ENSG00000168490 | PHYHIP | 8 | 22077222 | 22089854 |
| ENSG00000175309 | PHYKPL | 5 | 177635498 | 177659792 |
| ENSG00000131788 | PIAS3 | 1 | 145575233 | 145586546 |
| ENSG00000105229 | PIAS4 | 1 | 4007644 | 4039384 |
| ENSG00000197563 | PIGN | 1 | 59710800 | 59854351 |
| ENSG00000141506 | PIK3R5 | 1 | 8782233 | 8869029 |
| ENSG00000102096 | PIM2 | X | 48770459 | 48776301 |
| ENSG00000254093 | PINX1 | 8 | 10622473 | 10697394 |
| ENSG00000241878 | PISD | 2 | 32014477 | 32058418 |
| ENSG00000205038 | PKHD1L1 | 8 | 110374706 | 110542559 |
| ENSG00000057294 | PKP2 | 1 | 32943679 | 33049774 |
| ENSG00000144283 | PKP4 | 2 | 159313476 | 159539391 |
| ENSG00000176485 | PLA2G16 | 1 | 63340667 | 63384355 |
| ENSG00000181690 | PLAG1 | 8 | 57073463 | 57123883 |
| ENSG00000182621 | PLCB1 | 2 | 8112824 | 8949003 |
| ENSG00000161714 | PLCD3 | 1 | 43186335 | 43210721 |
| ENSG00000115896 | PLCL1 | 2 | 198669426 | 199437305 |
| ENSG00000115956 | PLEK | 2 | 68592305 | 68624585 |
| ENSG00000105559 | PLEKHA4 | 1 | 49340354 | 49371889 |
| ENSG00000052126 | PLEKHA5 | 1 | 19282648 | 19529334 |
| ENSG00000143850 | PLEKHA6 | 1 | 204187979 | 204346793 |
| ENSG00000187583 | PLEKHN1 | 1 | 901877 | 911245 |
| ENSG00000145632 | PLK2 | 5 | 57749809 | 57756087 |
| ENSG00000171566 | PLRG1 | 4 | 155456158 | 155471587 |
| ENSG00000120756 | PLS1 | 3 | 142315229 | 142432506 |
| ENSG00000102024 | PLS3 | X | 114795501 | 114885181 |
| ENSG00000130827 | PLXNA3 | X | 153686621 | 153701989 |
| ENSG00000196576 | PLXNB2 | 2 | 50713408 | 50746056 |
| ENSG00000176903 | PNMA1 | 1 | 74178494 | 74181128 |
| ENSG00000146278 | PNRC1 | 6 | 89790470 | 89794879 |
| ENSG00000102978 | POLR2C | 1 | 57496299 | 57505922 |
| ENSG00000185900 | POMK | 8 | 42948658 | 42978577 |
| ENSG00000105854 | PON2 | 7 | 95034175 | 95064510 |
| ENSG00000137709 | POU2F3 | 1 | 120107349 | 120190653 |
| ENSG00000180817 | PPA1 | 1 | 71962586 | 71993667 |
| ENSG00000141934 | PPAP2C | 1 | 281040 | 291393 |
| ENSG00000171497 | PPID | 4 | 159630286 | 159644548 |
| ENSG00000145725 | PPIP5K2 | 5 | 102455853 | 102548500 |
| ENSG00000118898 | PPL | 1 | 4932508 | 5010742 |
| ENSG00000100034 | PPM1F | 2 | 22273793 | 22307209 |
| ENSG00000077157 | PPP1R12B | 1 | 202317827 | 202561834 |
| ENSG00000115685 | PPP1R7 | 2 | 242088991 | 242123067 |
| ENSG00000105568 | PPP2R1A | 1 | 52693292 | 52730687 |
| ENSG00000156475 | PPP2R2B | 5 | 145967936 | 146464347 |
| ENSG00000011485 | PPP5C | 1 | 46850251 | 46896238 |
| ENSG00000196850 | PPTC7 | 1 | 110969120 | 111021125 |
| ENSG00000139174 | PRICKLE1 | 1 | 42852140 | 42984157 |
| ENSG00000106617 | PRKAG2 | 7 | 151253197 | 151574210 |
| ENSG00000154229 | PRKCA | 1 | 64298754 | 64806861 |
| ENSG00000065675 | PRKCQ | 1 | 6469105 | 6622263 |
| ENSG00000185532 | PRKG1 | 1 | 52750945 | 54058110 |
| ENSG00000126457 | PRMT1 | 1 | 50179043 | 50192286 |
| ENSG00000171867 | PRNP | 2 | 4666882 | 4682236 |
| ENSG00000184500 | PROS1 | 3 | 93591881 | 93692910 |
| ENSG00000112739 | PRPF4B | 6 | 4021501 | 4065217 |
| ENSG00000205352 | PRR13 | 1 | 53835389 | 53840429 |
| ENSG00000183530 | PRR14L | 2 | 32072242 | 32146126 |
| ENSG00000176532 | PRR15 | 7 | 29603427 | 29606911 |
| ENSG00000204469 | PRRC2A | 6 | 31588497 | 31605548 |
| ENSG00000005001 | PRSS22 | 1 | 2902728 | 2908171 |
| ENSG00000150687 | PRSS23 | 1 | 86502101 | 86663952 |
| ENSG00000105227 | PRX | 1 | 40899675 | 40919273 |
| ENSG00000156011 | PSD3 | 8 | 18384811 | 18942240 |
| ENSG00000112655 | PTK7 | 6 | 43044006 | 43129457 |
| ENSG00000188921 | PTPLAD2 | 9 | 20995306 | 21031635 |
| ENSG00000088179 | PTPN4 | 2 | 120517207 | 120741394 |
| ENSG00000081237 | PTPRC | 1 | 198607801 | 198726545 |
| ENSG00000132334 | PTPRE | 1 | 129705325 | 129884119 |
| ENSG00000142949 | PTPRF | 1 | 43990858 | 44089343 |
| ENSG00000144724 | PTPRG | 3 | 61547243 | 62283288 |
| ENSG00000152894 | PTPRK | 6 | 128289924 | 128841870 |
| ENSG00000139304 | PTPRQ | 1 | 80799774 | 81072802 |
| ENSG00000060656 | PTPRU | 1 | 29563028 | 29653325 |
| ENSG00000177469 | PTRF | 1 | 40554470 | 40575535 |
| ENSG00000091127 | PUS7 | 7 | 105080108 | 105162714 |
| ENSG00000100362 | PVALB | 2 | 37196728 | 37215523 |
| ENSG00000143217 | PVRL4 | 1 | 161040785 | 161059389 |
| ENSG00000100504 | PYGL | 1 | 51324609 | 51411454 |
| ENSG00000163564 | PYHIN1 | 1 | 158900586 | 158946844 |
| ENSG00000126838 | PZP | 1 | 9301436 | 9360966 |
| ENSG00000157869 | RAB28 | 4 | 13362978 | 13485989 |
| ENSG00000109113 | RAB34 | 1 | 27041299 | 27045447 |
| ENSG00000119318 | RAD23B | 9 | 110045418 | 110094475 |
| ENSG00000203722 | RAET1G | 6 | 150238014 | 150244257 |
| ENSG00000175097 | RAG2 | 1 | 36597124 | 36619829 |
| ENSG00000131831 | RAI2 | X | 17818169 | 17879457 |
| ENSG00000158987 | RAPGEF6 | 5 | 130759614 | 130970929 |
| ENSG00000165917 | RAPSN | 1 | 47459308 | 47470730 |
| ENSG00000172819 | RARG | 1 | 53604354 | 53626764 |
| ENSG00000145715 | RASA1 | 5 | 86563705 | 86687748 |
| ENSG00000100302 | RASD2 | 2 | 35936915 | 35950048 |
| ENSG00000068028 | RASSF1 | 3 | 50367219 | 50378411 |
| ENSG00000146587 | RBAK | 7 | 5085452 | 5109119 |
| ENSG00000102054 | RBBP7 | X | 16857406 | 16888537 |
| ENSG00000127993 | RBM48 | 7 | 92158087 | 92167319 |
| ENSG00000003756 | RBM5 | 3 | 50126341 | 50156454 |
| ENSG00000076067 | RBMS2 | 1 | 56915713 | 56984745 |
| ENSG00000117906 | RCN2 | 1 | 77223960 | 77242601 |
| ENSG00000079313 | REXO1 | 1 | 1815248 | 1848452 |
| ENSG00000127074 | RGS13 | 1 | 192605275 | 192629390 |
| ENSG00000155366 | RHOC | 1 | 113243728 | 113250056 |
| ENSG00000116574 | RHOU | 1 | 228870824 | 228882416 |
| ENSG00000176406 | RIMS2 | 8 | 104512976 | 105268322 |
| ENSG00000170881 | RNF139 | 8 | 125486979 | 125500155 |
| ENSG00000141576 | RNF157 | 1 | 74138534 | 74236454 |
| ENSG00000101236 | RNF24 | 2 | 3907956 | 3996229 |
| ENSG00000149489 | ROM1 | 1 | 62379194 | 62382592 |
| ENSG00000221817 | RP11-137L10.6 | 1 | 75255283 | 75279828 |
| ENSG00000271141 | RP11-17112.4 | 2 | 179481308 | 179481850 |
| ENSG00000132383 | RPA1 | 1 | 1732996 | 1803376 |
| ENSG00000156313 | RPGR | X | 38128416 | 38186817 |
| ENSG00000198755 | RPL10A | 6 | 35436185 | 35438562 |
| ENSG00000174748 | RPL15 | 3 | 23958036 | 23965183 |
| ENSG00000114391 | RPL24 | 3 | 101399935 | 101405626 |
| ENSG00000122406 | RPL5 | 1 | 93297582 | 93307481 |
| ENSG00000148303 | RPL7A | 9 | 136215069 | 136218281 |
| ENSG00000141425 | RPRD1A | 1 | 33564350 | 33647539 |
| ENSG00000163125 | RPRD2 | 1 | 150335567 | 150449042 |
| ENSG00000100784 | RPS6KA5 | 1 | 91336799 | 91526980 |
| ENSG00000170889 | RPS9 | 1 | 54704610 | 54752862 |
| ENSG00000155876 | RRAGA | 9 | 19049372 | 19051019 |
| ENSG00000025039 | RRAGD | 6 | 90074355 | 90121989 |
| ENSG00000126458 | RRAS | 1 | 50138549 | 50143458 |
| ENSG00000048392 | RRM2B | 8 | 103216730 | 103251346 |
| ENSG00000101282 | RSPO4 | 2 | 939095 | 982907 |
| ENSG00000143171 | RXRG | 1 | 165370159 | 165414433 |
| ENSG00000188643 | S100A16 | 1 | 153579362 | 153585621 |
| ENSG00000197956 | S100A6 | 1 | 153507075 | 153508720 |
| ENSG00000109929 | SC5D | 1 | 121163162 | 121179403 |
| ENSG00000139218 | SCAF11 | 1 | 46312914 | 46385903 |
| ENSG00000168077 | SCARA3 | 8 | 27491385 | 27534293 |
| ENSG00000136155 | SCEL | 1 | 78109809 | 78219398 |
| ENSG00000166922 | SCG5 | 1 | 32933877 | 32989299 |
| ENSG00000146285 | SCML4 | 6 | 108025308 | 108145521 |
| ENSG00000159307 | SCUBE1 | 2 | 43593289 | 43739394 |
| ENSG00000146197 | SCUBE3 | 6 | 35182190 | 35220856 |
| ENSG00000124145 | SDC4 | 2 | 43953928 | 43977064 |
| ENSG00000073578 | SDHA | 5 | 218356 | 256815 |
| ENSG00000146555 | SDK1 | 7 | 3341080 | 4308632 |
| ENSG00000100445 | SDR39U1 | 1 | 24908972 | 24912111 |
| ENSG00000075826 | SEC31B | 1 | 102246399 | 102289628 |
| ENSG00000085415 | SEH1L | 1 | 12947132 | 12987535 |
| ENSG00000186838 | SELV | 1 | 40005753 | 40011326 |
| ENSG00000153993 | SEMA3D | 7 | 84624869 | 84816171 |
| ENSG00000001617 | SEMA3F | 3 | 50192478 | 50226508 |
| ENSG00000138468 | SENP7 | 3 | 101043049 | 101232085 |
| ENSG00000183291 | SEP15 | 1 | 87328132 | 87380107 |
| ENSG00000109618 | SEPSECS | 4 | 25121627 | 25162204 |
| ENSG00000168385 | SEPT2 | 2 | 242254515 | 242293442 |
| ENSG00000178980 | SEPW1 | 1 | 48281829 | 48287943 |
| ENSG00000129158 | SERGEF | 1 | 17809595 | 18034709 |
| ENSG00000197249 | SERPINA1 | 1 | 94843084 | 94857030 |
| ENSG00000197019 | SERTAD1 | 1 | 40927499 | 40931932 |
| ENSG00000139718 | SETD1B | 1 | 122242086 | 122270562 |
| ENSG00000168066 | SF1 | 1 | 64532078 | 64546258 |
| ENSG00000115128 | SF3B14 | 2 | 24290454 | 24299313 |
| ENSG00000087365 | SF3B2 | 1 | 65818200 | 65836779 |
| ENSG00000189091 | SF3B3 | 1 | 70557691 | 70608820 |
| ENSG00000061936 | SFSWAP | 1 | 132195626 | 132284282 |
| ENSG00000163069 | SGCB | 4 | 52886872 | 52904648 |
| ENSG00000127990 | SGCE | 7 | 94214542 | 94285521 |
| ENSG00000164023 | SGMS2 | 4 | 108745719 | 108836203 |
| ENSG00000104611 | SH2D4A | 8 | 19171128 | 19253729 |
| ENSG00000160691 | SHC1 | 1 | 154934774 | 154946871 |
| ENSG00000169291 | SHE | 1 | 154442248 | 154474589 |
| ENSG00000138606 | SHF | 1 | 45459412 | 45493373 |
| ENSG00000158352 | SHROOM4 | X | 50334647 | 50557302 |
| ENSG00000181788 | SIAH2 | 3 | 150458914 | 150481264 |
| ENSG00000147955 | SIGMAR1 | 9 | 34634719 | 34637806 |
| ENSG00000162739 | SLAMF6 | 1 | 160454820 | 160493052 |
| ENSG00000120519 | SLC10A7 | 4 | 147175127 | 147443123 |
| ENSG00000064651 | SLC12A2 | 5 | 127419458 | 127525380 |
| ENSG00000155380 | SLC16A1 | 1 | 113454469 | 113499635 |
| ENSG00000168679 | SLC16A4 | 1 | 110905470 | 110933704 |
| ENSG00000119899 | SLC17A5 | 6 | 74303102 | 74363878 |
| ENSG00000259803 | SLC22A31 | 1 | 89262406 | 89268072 |
| ENSG00000102743 | SLC25A15 | 1 | 41363548 | 41384247 |
| ENSG00000155287 | SLC25A28 | 1 | 101370282 | 101380366 |
| ENSG00000125434 | SLC25A35 | 1 | 8191081 | 8198661 |
| ENSG00000140284 | SLC27A2 | 1 | 50474393 | 50528592 |
| ENSG00000113396 | SLC27A6 | 5 | 127873706 | 128369335 |
| ENSG00000160326 | SLC2A6 | 9 | 136336217 | 136344259 |
| ENSG00000152683 | SLC30A6 | 2 | 32390933 | 32449448 |
| ENSG00000136868 | SLC31A1 | 9 | 115983808 | 116028674 |
| ENSG00000136867 | SLC31A2 | 9 | 115913222 | 115926417 |
| ENSG00000157765 | SLC34A2 | 4 | 25656923 | 25680370 |
| ENSG00000121073 | SLC35B1 | 1 | 47778305 | 47786376 |
| ENSG00000110660 | SLC35F2 | 1 | 107661717 | 107799019 |
| ENSG00000183780 | SLC35F3 | 1 | 234040679 | 234460262 |
| ENSG00000141424 | SLC39A6 | 1 | 33688495 | 33709348 |
| ENSG00000134802 | SLC43A3 | 1 | 57174427 | 57195053 |
| ENSG00000004939 | SLC4A1 | 1 | 42325753 | 42345509 |
| ENSG00000080493 | SLC4A4 | 4 | 72053003 | 72437804 |
| ENSG00000169241 | SLC50A1 | 1 | 155107820 | 155111329 |
| ENSG00000140675 | SLC5A2 | 1 | 31494323 | 31502181 |
| ENSG00000103064 | SLC7A6 | 1 | 68298433 | 68335722 |
| ENSG00000145147 | SLIT2 | 4 | 20254883 | 20622184 |
| ENSG00000163681 | SLMAP | 3 | 57741177 | 57914895 |
| ENSG00000124107 | SLPI | 2 | 43880880 | 43883205 |
| ENSG00000137776 | SLTM | 1 | 59171244 | 59225852 |
| ENSG00000157106 | SMG1 | 1 | 18816175 | 18937776 |
| ENSG00000163683 | SMIM14 | 4 | 39547950 | 39640710 |
| ENSG00000130768 | SMPDL3B | 1 | 28261504 | 28285668 |
| ENSG00000122692 | SMU1 | 9 | 33041762 | 33076665 |
| ENSG00000145335 | SNCA | 4 | 90645250 | 90759466 |
| ENSG00000173267 | SNCG | 1 | 88718375 | 88723017 |
| ENSG00000212443 | SNORA53 | 1 | 98993413 | 98993661 |
| ENSG00000163788 | SNRK | 3 | 43328004 | 43466256 |
| ENSG00000028528 | SNX1 | 1 | 64386322 | 64438289 |
| ENSG00000002919 | SNX11 | 1 | 46180719 | 46200436 |
| ENSG00000147164 | SNX12 | X | 70279094 | 70288273 |
| ENSG00000167208 | SNX20 | 1 | 50700211 | 50715264 |
| ENSG00000157734 | SNX22 | 1 | 64443914 | 64449680 |
| ENSG00000109762 | SNX25 | 4 | 186125391 | 186291339 |
| ENSG00000173548 | SNX33 | 1 | 75940247 | 75954642 |
| ENSG00000089006 | SNX5 | 2 | 17922241 | 17949623 |
| ENSG00000198944 | SOWAHA | 5 | 132149033 | 132152488 |
| ENSG00000124766 | SOX4 | 6 | 21593972 | 21598847 |
| ENSG00000172845 | SP3 | 2 | 174771187 | 174830430 |
| ENSG00000196141 | SPATS2L | 2 | 201170604 | 201346986 |
| ENSG00000166145 | SPINT1 | 1 | 41136216 | 41150405 |
| ENSG00000198369 | SPRED2 | 2 | 65537985 | 65659771 |
| ENSG00000164056 | SPRY1 | 4 | 124317950 | 124324910 |
| ENSG00000187678 | SPRY4 | 5 | 141689992 | 141706020 |
| ENSG00000197694 | SPTAN1 | 9 | 131314866 | 131395941 |
| ENSG00000090054 | SPTLC1 | 9 | 94794281 | 94877666 |
| ENSG00000075142 | SRI | 7 | 87834433 | 87856308 |
| ENSG00000167881 | SRP68 | 1 | 74035184 | 74068734 |
| ENSG00000135250 | SRPK2 | 7 | 104751151 | 105039755 |
| ENSG00000116350 | SRSF4 | 1 | 29474255 | 29508499 |
| ENSG00000145687 | SSBP2 | 5 | 80708840 | 81047616 |
| ENSG00000149136 | SSRP1 | 1 | 57093459 | 57103351 |
| ENSG00000160075 | SSU72 | 1 | 1477053 | 1510249 |
| ENSG00000157350 | ST3GAL2 | 1 | 70413338 | 70473140 |
| ENSG00000115525 | ST3GAL5 | 2 | 86066267 | 86116137 |
| ENSG00000167323 | STIM1 | 1 | 3875757 | 4114439 |
| ENSG00000169302 | STK32A | 5 | 146614526 | 146767415 |
| ENSG00000165283 | STOML2 | 9 | 35099888 | 35103154 |
| ENSG00000137868 | STRA6 | 1 | 74471807 | 74504608 |
| ENSG00000104915 | STX10 | 1 | 13254872 | 13261197 |
| ENSG00000124222 | STX16 | 2 | 57226328 | 57254582 |
| ENSG00000111450 | STX2 | 1 | 131274145 | 131323811 |
| ENSG00000177688 | SUMO4 | 6 | 149721495 | 149722177 |
| ENSG00000102710 | SUPT20H | 1 | 37583449 | 37633850 |
| ENSG00000196235 | SUPT5H | 1 | 39926796 | 39967310 |
| ENSG00000148291 | SURF2 | 9 | 136223428 | 136228045 |
| ENSG00000099994 | SUSD2 | 2 | 24577227 | 24585078 |
| ENSG00000159164 | SV2A | 1 | 149874870 | 149889434 |
| ENSG00000173928 | SWSAP1 | 1 | 11485361 | 11487627 |
| ENSG00000171992 | SYNPO | 5 | 149980642 | 150038782 |
| ENSG00000006114 | SYNRG | 1 | 35874900 | 35969544 |
| ENSG00000147041 | SYTL5 | X | 37865835 | 37988072 |
| ENSG00000184292 | TACSTD2 | 1 | 59041099 | 59043166 |
| ENSG00000064995 | TAF11 | 6 | 34845555 | 34855866 |
| ENSG00000103168 | TAF1C | 1 | 84211458 | 84220669 |
| ENSG00000165632 | TAF3 | 1 | 7860467 | 8058590 |
| ENSG00000144559 | TAMM41 | 3 | 11831916 | 11888393 |
| ENSG00000183597 | TANGO2 | 2 | 20004537 | 20053449 |
| ENSG00000113838 | TBCCD1 | 3 | 186263862 | 186288332 |
| ENSG00000176896 | TCEANC | X | 13671225 | 13700083 |
| ENSG00000116205 | TCEANC2 | 1 | 54519260 | 54578192 |
| ENSG00000139437 | TCHP | 1 | 110338069 | 110421646 |
| ENSG00000182134 | TDRKH | 1 | 151742583 | 151763892 |
| ENSG00000205356 | TECPR1 | 7 | 97843936 | 97881563 |
| ENSG00000009694 | TENM1 | X | 123509753 | 124097666 |
| ENSG00000115112 | TFCP2L1 | 2 | 121974163 | 122042783 |
| ENSG00000163235 | TGFA | 2 | 70674412 | 70781325 |
| ENSG00000140682 | TGFB1I1 | 1 | 31482906 | 31489281 |
| ENSG00000092969 | TGFB2 | 1 | 218519577 | 218617961 |
| ENSG00000092295 | TGM1 | 1 | 24718320 | 24733638 |
| ENSG00000169231 | THBS3 | 1 | 155165379 | 155178842 |
| ENSG00000151365 | THRSP | 1 | 77774907 | 77779397 |
| ENSG00000102265 | TIMP1 | X | 47441712 | 47446188 |
| ENSG00000035862 | TIMP2 | 1 | 76849059 | 76921469 |
| ENSG00000163659 | TIPARP | 3 | 156391024 | 156424559 |
| ENSG00000119139 | TJP2 | 9 | 71736209 | 71870124 |
| ENSG00000169908 | TM4SF1 | 3 | 149086809 | 149095652 |
| ENSG00000169903 | TM4SF4 | 3 | 149191761 | 149221068 |
| ENSG00000144868 | TMEM108 | 3 | 132757235 | 133116636 |
| ENSG00000011638 | TMEM159 | 1 | 21169698 | 21191937 |
| ENSG00000164180 | TMEM161B | 5 | 87485450 | 87565293 |
| ENSG00000152128 | TMEM163 | 2 | 135213330 | 135476570 |
| ENSG00000157600 | TMEM164 | X | 109245859 | 109425962 |
| ENSG00000187713 | TMEM203 | 9 | 140098534 | 140100090 |
| ENSG00000131634 | TMEM204 | 1 | 1578689 | 1605581 |
| ENSG00000186501 | TMEM222 | 1 | 27648651 | 27662891 |
| ENSG00000106609 | TMEM248 | 7 | 66386212 | 66423538 |
| ENSG00000112697 | TMEM30A | 6 | 75962640 | 75994684 |
| ENSG00000163900 | TMEM41A | 3 | 185194284 | 185216845 |
| ENSG00000145014 | TMEM44 | 3 | 194308402 | 194354418 |
| ENSG00000180694 | TMEM64 | 8 | 91634223 | 91803860 |
| ENSG00000163472 | TMEM79 | 1 | 156252726 | 156262976 |
| ENSG00000103978 | TMEM87A | 1 | 42502730 | 42565861 |
| ENSG00000153214 | TMEM87B | 2 | 112812800 | 112876895 |
| ENSG00000006042 | TMEM98 | 1 | 31254928 | 31272124 |
| ENSG00000137648 | TMPRSS4 | 1 | 117947753 | 117992605 |
| ENSG00000187045 | TMPRSS6 | 2 | 37461476 | 37505603 |
| ENSG00000034510 | TMSB10 | 2 | 85132749 | 85133795 |
| ENSG00000041982 | TNC | 9 | 117782806 | 117880536 |
| ENSG00000006327 | TNFRSF12A | 1 | 3068446 | 3072384 |
| ENSG00000048462 | TNFRSF17 | 1 | 12058964 | 12061925 |
| ENSG00000067182 | TNFRSF1A | 1 | 6437923 | 6451280 |
| ENSG00000173273 | TNKS | 8 | 9413424 | 9639856 |
| ENSG00000183864 | TOB2 | 2 | 41829496 | 41843027 |
| ENSG00000132773 | TOE1 | 1 | 45805342 | 45809647 |
| ENSG00000173726 | TOMM20 | 1 | 235272651 | 235292251 |
| ENSG00000177302 | TOP3A | 1 | 18174742 | 18218321 |
| ENSG00000169905 | TOR1AIP2 | 1 | 179809102 | 179846938 |
| ENSG00000160404 | TOR2A | 9 | 130493803 | 130497604 |
| ENSG00000143514 | TP53BP2 | 1 | 223967601 | 224033674 |
| ENSG00000170638 | TRABD | 2 | 50624344 | 50638027 |
| ENSG00000056972 | TRAF3IP2 | 6 | 111877657 | 111927481 |
| ENSG00000175104 | TRAF6 | 1 | 36508577 | 36531822 |
| ENSG00000160218 | TRAPPC10 | 2 | 45432200 | 45526433 |
| ENSG00000171853 | TRAPPC12 | 2 | 3383446 | 3488865 |
| ENSG00000196655 | TRAPPC4 | 1 | 118889142 | 118896164 |
| ENSG00000204599 | TRIM39 | 6 | 30294256 | 30311506 |
| ENSG00000183718 | TRIM52 | 5 | 180681417 | 180688119 |
| ENSG00000166436 | TRIM66 | 1 | 8633584 | 8693413 |
| ENSG00000173113 | TRMT112 | 1 | 64083932 | 64085556 |
| ENSG00000072315 | TRPC5 | X | 111017543 | 111326004 |
| ENSG00000102804 | TSC22D1 | 1 | 45007655 | 45151283 |
| ENSG00000157514 | TSC22D3 | X | 106956451 | 107020572 |
| ENSG00000179981 | TSHZ1 | 1 | 72922710 | 73001905 |
| ENSG00000187189 | TSPYL4 | 6 | 116571151 | 116575261 |
| ENSG00000182670 | TTC3 | 2 | 38445526 | 38575413 |
| ENSG00000214021 | TTLL3 | 3 | 9849770 | 9896822 |
| ENSG00000188229 | TUBB4B | 9 | 140135665 | 140138159 |
| ENSG00000104723 | TUSC3 | 8 | 15274724 | 15624158 |
| ENSG00000117862 | TXNDC12 | 1 | 52485803 | 52521843 |
| ENSG00000092445 | TYRO3 | 1 | 41849873 | 41871536 |
| ENSG00000117143 | UAP1 | 1 | 162531323 | 162569627 |
| ENSG00000184787 | UBE2G2 | 2 | 46188955 | 46221934 |
| ENSG00000103275 | UBE2I | 1 | 1355548 | 1377019 |
| ENSG00000215218 | UBE2QL1 | 5 | 6448736 | 6495022 |
| ENSG00000162543 | UBXN10 | 1 | 20512578 | 20522541 |
| ENSG00000158062 | UBXN11 | 1 | 26607819 | 26644854 |
| ENSG00000116750 | UCHL5 | 1 | 192981380 | 193029237 |
| ENSG00000143222 | UFC1 | 1 | 161122566 | 161128646 |
| ENSG00000109814 | UGDH | 4 | 39500375 | 39529931 |
| ENSG00000131015 | ULBP2 | 6 | 150263136 | 150270371 |
| ENSG00000177169 | ULK1 | 1 | 132379196 | 132407712 |
| ENSG00000151461 | UPF2 | 1 | 11962021 | 12085169 |
| ENSG00000125351 | UPF3B | X | 118967985 | 118986961 |
| ENSG00000077254 | USP33 | 1 | 78161672 | 78225537 |
| ENSG00000132952 | USPL1 | 1 | 31191830 | 31233686 |
| ENSG00000156697 | UTP14A | X | 129040097 | 129063737 |
| ENSG00000163945 | UVSSA | 4 | 1341054 | 1381837 |
| ENSG00000168140 | VASN | 1 | 4421849 | 4433529 |
| ENSG00000100483 | VCPKMT | 1 | 50575350 | 50583318 |
| ENSG00000187650 | VMAC | 1 | 5904869 | 5910864 |
| ENSG00000139722 | VPS37B | 1 | 123349882 | 123380991 |
| ENSG00000156931 | VPS8 | 3 | 184529931 | 184770402 |
| ENSG00000165633 | VSTM4 | 1 | 50222290 | 50323554 |
| ENSG00000151532 | VTI1A | 1 | 114206756 | 114578503 |
| ENSG00000179403 | VWA1 | 1 | 1370241 | 1378262 |
| ENSG00000110002 | VWA5A | 1 | 123986069 | 124018428 |
| ENSG00000204396 | VWA7 | 6 | 31733367 | 31745108 |
| ENSG00000015285 | WAS | X | 48534985 | 48549818 |
| ENSG00000196998 | WDR45 | X | 48929385 | 48958108 |
| ENSG00000070540 | WIPI1 | 1 | 66417089 | 66453654 |
| ENSG00000142279 | WTIP | 1 | 34971874 | 34997258 |
| ENSG00000182489 | XKRX | X | 100168431 | 100184422 |
| ENSG00000143324 | XPR1 | 1 | 180601140 | 180859387 |
| ENSG00000079246 | XRCC5 | 2 | 216972187 | 217071026 |
| ENSG00000177494 | ZBED2 | 3 | 111311747 | 111314290 |
| ENSG00000126804 | ZBTB1 | 1 | 64970430 | 65000408 |
| ENSG00000205189 | ZBTB10 | 8 | 81397854 | 81438500 |
| ENSG00000177485 | ZBTB33 | X | 119384607 | 119392253 |
| ENSG00000168826 | ZBTB49 | 4 | 4291924 | 4323513 |
| ENSG00000104427 | ZC2HC1A | 8 | 79578282 | 79632000 |
| ENSG00000122299 | ZC3H7A | 1 | 11844442 | 11891123 |
| ENSG00000144161 | ZC3H8 | 2 | 112969102 | 113012713 |
| ENSG00000174460 | ZCCHC12 | X | 117957753 | 117960931 |
| ENSG00000186908 | ZDHHC17 | 1 | 77157368 | 77247476 |
| ENSG00000156599 | ZDHHC5 | 1 | 57435219 | 57468659 |
| ENSG00000153786 | ZDHHC7 | 1 | 85007787 | 85045141 |
| ENSG00000133858 | ZFC3H1 | 1 | 72003252 | 72061505 |
| ENSG00000152518 | ZFP36L2 | 2 | 43449541 | 43453748 |
| ENSG00000039319 | ZFYVE16 | 5 | 79703832 | 79775169 |
| ENSG00000172667 | ZMAT3 | 3 | 178735011 | 178790067 |
| ENSG00000165061 | ZMAT4 | 8 | 40388109 | 40755352 |
| ENSG00000163867 | ZMYM6 | 1 | 35449523 | 35497569 |
| ENSG00000172262 | ZNF131 | 5 | 43065278 | 43192123 |
| ENSG00000256294 | ZNF225 | 1 | 44616334 | 44637027 |
| ENSG00000159917 | ZNF235 | 1 | 44732882 | 44809199 |
| ENSG00000158805 | ZNF276 | 1 | 89786808 | 89807311 |
| ENSG00000160961 | ZNF333 | 1 | 14800613 | 14844558 |
| ENSG00000130684 | ZNF337 | 2 | 25654851 | 25677477 |
| ENSG00000189180 | ZNF33A | 1 | 38299578 | 38354016 |
| ENSG00000113761 | ZNF346 | 5 | 176449697 | 176508190 |
| ENSG00000256683 | ZNF350 | 1 | 52467596 | 52490109 |
| ENSG00000197024 | ZNF398 | 7 | 148823508 | 148880116 |
| ENSG00000215421 | ZNF407 | 1 | 72265106 | 72777627 |
| ENSG00000133250 | ZNF414 | 1 | 8575462 | 8579048 |
| ENSG00000173480 | ZNF417 | 1 | 58411664 | 58427978 |
| ENSG00000183621 | ZNF438 | 1 | 31109136 | 31320866 |
| ENSG00000185219 | ZNF445 | 3 | 44481262 | 44519162 |
| ENSG00000197016 | ZNF470 | 1 | 57078880 | 57100279 |
| ENSG00000101493 | ZNF516 | 1 | 74069644 | 74207146 |
| ENSG00000074657 | ZNF532 | 1 | 56529832 | 56653712 |
| ENSG00000258405 | ZNF578 | 1 | 52956829 | 53015407 |
| ENSG00000198466 | ZNF587 | 1 | 58361225 | 58376480 |
| ENSG00000197343 | ZNF655 | 7 | 99156029 | 99174076 |
| ENSG00000196757 | ZNF700 | 1 | 12035883 | 12061588 |
| ENSG00000181135 | ZNF707 | 8 | 144766622 | 144796068 |
| ENSG00000196456 | ZNF775 | 7 | 150065879 | 150109558 |
| ENSG00000198556 | ZNF789 | 7 | 99070464 | 99101273 |
| ENSG00000204524 | ZNF805 | 1 | 57751973 | 57766503 |
| ENSG00000178917 | ZNF852 | 3 | 44540462 | 44552128 |
| ENSG00000106479 | ZNF862 | 7 | 149535456 | 149564568 |
| ENSG00000070476 | ZXDC | 3 | 126156444 | 126194762 |
| ENSG00000074755 | ZZEF1 | 1 | 3907739 | 4046314 |

### Example 9. Statistical Analysis

Statistical analyses were performed using R statistical software version 3.2.3. Continuous variables were compared using t test, and categorical variables were compared using Fisher exact test. Test performance was evaluated using sensitivity, specificity, and NPV and PPV based on established methods. All confidence intervals are 2-sided 95% CIs and were computed using the exact binomial test. Test performance comparison between the GSC and GEC was done using McNemar χ² test on the matched data set. Significance level in differential gene expression analysis is reported using a false discovery rate-adjusted P value. Two-sided P values less than 0.05 were used to declare significance.

### RESULTS

FNA samples that previously validated the GEC were used to independently validate the GSC. The earlier GEC validation samples were derived from 4812 nodule aspirations prospectively collected from 3789 patients at 49 clinical sites in the United States over a 2-year period. Of the 210 validation samples with corresponding Bethesda III or IV cytology and blinded postoperative consensus histopathology diagnoses, 191 (91.0%) had sufficient residual RNA for GSC testing. These samples from cytologically indeterminate nodules constituted the blinded primary test set.

The previously established thyroid nodule cytological diagnosis was used again. Patient demographic characteristics and baseline data are shown in Table 4. Age, sex, clinical risk factors, nodule size, histology subtype (Table 5), number of FNA passes, prevalence of malignancy (Table 6), and proportion of samples collected at community centers did not differ significantly between the primary study population (n=191) and the GEC clinical validation cohort of samples (n=210), consistent with unbiased drop out.

TABLE 4
Baseline demographic and clinical characteristics of the study cohort^{a}.

| Variable | GEC Validation | GSC Validation |
|---|---|---|
| Total, No. | | |
| Samples | 210 | 191 |
| Patients | 199 | 183 |
| Type of study site, No. (%) of samples | | |
| Academic | 76 (36.2) | 65 (34.0) |
| Community | 134 (63.8) | 126 (66.0) |
| No. of fine-needle aspiration passes, No. (%) of samples | | |
| 1 | 88 (41.9) | 73 (38.2) |
| 2 | 122 (58.1) | 118 (61.8) |
| Age of patients, mean (range), y | 51.2 (22.0-85.0) | 51.7 (22.0-85.0) |
| Male | 46 (23.1) | 41 (22.4) |
| Female | 153 (76.9) | 142 (77.6) |
| Risk factors, No. (%) of patients | | |
| Radiation exposure to head, neck, or both | 7 (3.5) | 5 (2.7) |
| Family history of thyroid cancer | 14 (7.0) | 13 (7.1) |
| Nodule | | |
| Size of ultrasonography, median (range), cm | 2.5 (1.0-9.1) | 2.6 (1.0-9.1) |
| Size group, No. (%) of nodules, cm | | |
| 1.00-1.99 | 69 (32.9) | 60 (31.4) |
| 2.00-2.99 | 62 (29.5) | 60 (31.4) |
| 3.00-3.99 | 42 (20.0) | 37 (19.4) |
| ≥4.00 | 37 (17.6) | 34 (17.8) |

Abbreviations:
GEC, gene expression classifier;
GSC, genomic sequencing classifier
^{a}Statistical tests were performed to compare the 19 nodules in the GEC validation that were excluded in the GSC validation because of insufficient RNA quantity. The 2 groups differ only on the number of fine-needle aspiration passes, which is not unexpected, as only samples with sufficient remaining RNA were included in the GSC evaluation.

TABLE 5
Histology subtype comparison between validation cohorts.

| Histology Subtype Group | GEC (N = 210) | GSC (N = 191) | P-value |
|---|---|---|---|
| BFN, HN | 63 | 54 | 0.47 |
| FA | 56 | 54 | |
| FT-UMP, WDT-UMP | 18 | 17 | |
| HCA | 19 | 17 | |
| CLT, HT | 2 | 2 | |
| HTA | 1 | 1 | |
| PTC, PTC-TCV | 18 | 17 | |
| FVPTC | 12 | 11 | |
| HCC-c, HCC-v | 9 | 19 | |
| FC-c, FC-v, WDC-NOS | 9 | 7 | |
| PDC, ML, MTC | 3 | 2 | |

P-value is from a test comparing the 191 GSC nodules with the 19 nodules in the GEC validation that were excluded in the GSC validation due to insufficient RNA quantity. Histology subtype abbreviations: BFN-benign follicular nodule, HN-hyperplastic nodule, FA follicular adenoma, FT-UMP-follicular tumor of uncertain malignant potential, WDT-UMP well differentiated tumor of uncertain malignant potential, HCA-Hürthle cell adenoma, CLT chronic lymphocytic thyroiditis, HT-Hashimoto's thyroiditis, HTA-hyalinizing trabecular adenoma, PTC-papillary thyroid cancer, PTC-TCV-papillary thyroid cancer tall cell variant, FVPTC-papillary thyroid cancer follicular variant, HCC-c-Hürthle cell carcinoma capsular invasion, HCC-v-Hürthle cell carcinoma vascular invasion, FC-c-follicular carcinoma capsular invasion, FC-v-follicular carcinoma vascular invasion, WDC-NOS-well differentiated carcinoma not otherwise specified, PDC-poorly differentiated carcinoma, ML malignant lymphoma, MTC-medullary thyroid cancer

TABLE 6
Prevalence of malignancy between validation cohorts.

| Histologic Label | GEC (N = 210) | GSC (N = 191) | P-value |
|---|---|---|---|
| Benign | 159 | 145 | 1.00 |
| Malignant | 51 | 46 | |
| Cancer prevalence | 24.3% | 24.1% | |

P-value is from a test comparing the 191 GSC nodules with the 19 nodules in the GEC validation that were excluded in the GSC validation due to insufficient RNA quantity.

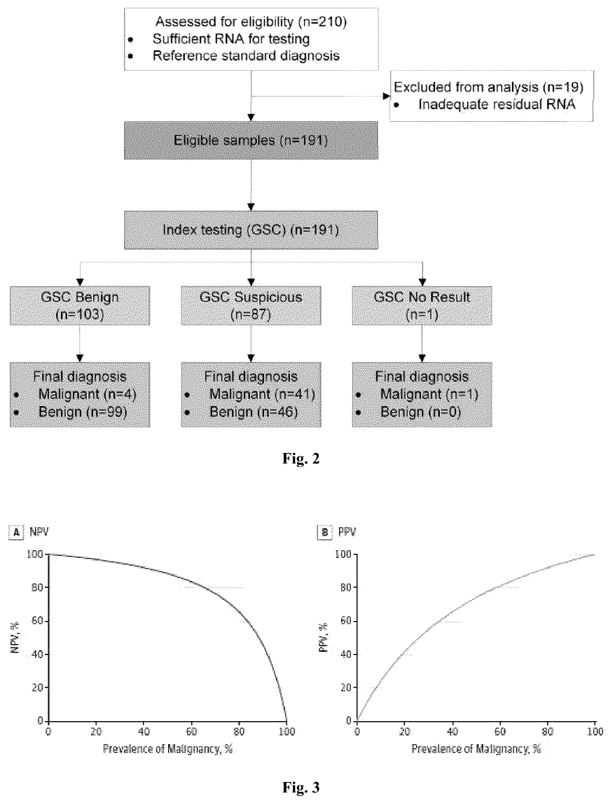

The Standards for Reporting of Diagnostic Accuracy Studies was developed to improve the quality of reporting diagnostic accuracy studies. FIG. 2 shows the flow of samples through the study in a Standards for Reporting of Diagnostic Accuracy Studies diagram. Of these 191 indeterminate FNAs, 46 (24.1%) were diagnosed as malignant by an expert surgical histopathology panel who were blinded to all cytologic and genomic results and to the local histopathology diagnosis. Results are reported in the order of testing through the GSC test system (FIG. 1). Initially, all GSC samples are tested for RNA quantity and quality. None of the 191 samples failed. Subsequently, the GSC aimed to identify nodules composed of parathyroid tissue, those with MTC, and those with a BRAF V600E mutation or RET/PTC1 or RET/PTC3 fusion. Samples testing positive forthese are included in performance calculations described below, except for samples testing positive for parathyroid tissue, as this result does not indicate a benign or malignant etiology. Among the 191 samples, positive results for parathyroid, MTC, BRAF, and RET/PTC occurred in 0, 1, 3, and 0 samples, respectively. All MTC and BRAF V600E results were concordant with reference methods. After this testing, samples were evaluated for follicular cell content by the follicular content index classifier. One sample, negative for the above results, was deemed to have inadequate follicular content and therefore was assigned no result. This sample was excluded from subsequent analyses, leaving 190 samples. Table 7 summarizes clinical performance characteristics for Bethesda III and IV nodules.

TABLE 7
Performance of the Genomic Sequencing Classifier (GSC) According to
the Final Histopathological Diagnoses and Cytopathological Category.

| | Reference Standard, % (95% CI) | | |
|---|---|---|---|
| GSC Result | Malignant | | Benign |
| Performance across the primary test set of Bethesda III | | | |
| and IV indeterminate nodules (n = 190) | | | |
| Suspicious, No./total No. | 41/45 | | 46/145 |
| Benign, No./total No. | 4/45 | | 99/145 |
| Sensitivity | 91.1 | (79-98) | |
| Specificity | 68.3 | (60-76) | |
| NPV | 96.1 | (90-99) | |
| PPV | 47.1 | (36-58) | |
| Prevalence of malignant lesions, % | 23.7 | | |
| Bethesda III: atypia of undermined significance/follicular | | | |
| lesion of undetermined significance (n = 114 [60.0%]) | | | |
| Suspicious, No./total No. | 26/28 | | 25/86 |
| Benign, No./total No. | 2/28 | | 61/86 |
| Sensitivity | 92.9 | (76-99) | |
| Specificity | 70.9 | (60-80) | |
| NPV | 96.8 | (89-100) | |
| PPV | 51.0 | (37-65) | |
| Prevalence of malignant lesions, % | 24.6 | | |
| Bethesda IV: follicular of Hürthle cell neoplasm or | | | |
| suspicious for follicular neoplasm (n = 76 [40.0%]) | | | |
| Suspicious, No./total No. | 15/17 | | 21/59 |
| Benign, No./total No. | 2/17 | | 38/59 |
| Sensitivity | 88.2 | (64-99) | |
| Specificity | 64.4 | (51-76) | |
| NPV | 95.0 | (83-99) | |
| PPV | 41.7 | (26-59) | |
| Prevalence of malignant lesions, % | 22.4 | | |
| Performance across the secondary test set of Bethesda II, | | | |
| V, and VI nodules (n = 61)^{a} | | | |
| Suspicious, No./total No. | 34/34 | | 7/26 |
| Benign, No./total No. | 0/34 | | 19/26 |
| Sensitivity | 100 | (90-100) | |
| Specificity | 73.1 | (52-88) | |
| NPV | 100 | (82-100) | |
| PPV | 82.9 | (68-93) | |
| Prevalence of malignant lesions, % | 56.7 | | |
| Bethesda II: cytopathologically benign (n = 19 [31.1%])^{a} | | | |
| Suspicious, No./total No. | 2.2 | | 2/16 |
| Benign, No./total No. | 2/0 | | 14/16 |
| Sensitivity | 100 | (16-100) | |
| Specificity | 87.5 | (62-98) | |
| NPV | 100 | (77-100) | |
| PPV | 50.0 | (7-93) | |
| Prevalence of malignant lesions, % | 11.1 | | |
| Bethesda V: suspicious for malignancy (n = 23 [37.7%]) | | | |
| Suspicious, No./total No. | 13/13 | | 5/10 |
| Benign, No./total No. | 0/13 | | 5/10 |
| Sensitivity | 100 | (75-100) | |
| Specificity | 50.0 | (19-81) | |
| NPV | 100 | (48-100) | |
| PPV | 72.2 | (47-90) | |
| Prevalence of malignant lesions, % | 56.5 | | |
| Bethesda VI: cytopathologically malignant | | | |
| (n = 19 [31.1%]) | | | |
| Suspicious, No./total No. | 19/19 | | 0/0 |
| Benign, No./total No. | 0/19 | | 0/0 |
| Sensitivity | 100 | (82-100) | |
| PPV | 100 | (82-100) | |

| | |
|---|---|
| Prevalence of malignant lesions, % | 100 |

Abbreviations:
NVP, negative predictive value;
PPV, positive predictive value
^{a}One sample has no result because of low follicular content that is not summarized in the table.

The GSC correctly identified 41 of N45 malignant samples as suspicious, yielding a sensitivity of 91.1% (95% CI, 79-98), and 99 of 145 nonmalignant samples were correctly identified as benign by the GSC, yielding a specificity of 68.3% (95% CI, 6076). Among Bethesda III and IV samples, the NPV was 96.1% (95% CI, 90-99) and the PPV was 47.1% (95% CI, 36-58). Performance of the GSC was similar between Bethesda III and IV categories (Table 7).

Among the 190 Bethesda III and IV samples, 17 (8.9%) were histologically Hürthle cell adenomas and 9 (4.7%) were Hürthle cell carcinomas, while 164 samples (86.3%) were histologically non-Hürthle. For samples with Hürthle histology, the sensitivity was 88.9% (95% CI, 52-100) and the specificity was 58.8% (95% CI, 33-82). For samples with non-Hürthle histology, the sensitivity was 91.7% (95% CI, 78-98) and the specificity was 69.5% (95% CI, 61-77).

A wide variety of malignant subtypes were correctly classified as suspicious (Table 8). Four false-negative cases occurred (Table 9). Patient age or sex, malignancy subtype, or nodule size by ultrasonography or on histopathology were assessed to determine whether they associated with false-negative cases, and none were. The performance of the GSC in secondary analyses of nodules with Bethesda 11, V, or VI cytopathology are reported in Table 7. Among the entire secondary analysis group, the GSC sensitivity was 100% (95%₀C1, 90-100) and the specificity was 73.1% (95%₀C1, 52-88).

TABLE 8
Performance of Genomic Sequencing Classifier
(GSC) According to Histopathological Subtype.

| | | Result with GSC, |
|---|---|---|
| | | Benign, No./ |
| | | Suspicious, |
| Histopathological Subtype | Nodules, No. (%) | No. |
| Benign | | |
| Total, No. | 145 | NA |
| Benign follicular nodule | 49 (33.8) | 33/11 |
| Hyperplastic nodule | 5 (3.4) | 5/0 |
| Follicular adenoma | 54 (37.2) | 37/17 |
| Follicular tumor of uncertain malignant potential | 9 (6.2) | 4/5 |
| Well-differentiated tumor of uncertain malignant | 8 (5.5) | 4/4 |
| potential | | |
| Hürthle cell adenoma | 17 (11.7) | 10.7 |
| Chronic lymphocytic thyroiditis | 2 (1.4) | 1/1 |
| Hyalinizing trabecular adenoma | 1 (0.7) | 0/1 |
| Malignant | | |
| Total, No. | 45 | NA |
| Papillary thyroid carcinoma | 15 (33.3) | 2/13 |
| Tall-cell variant | 1 (2.2) | 0/1 |
| Follicular carcinoma | 11 (24.4) | 1/10 |
| Hurthle cell carcinoma^{a} | 9 (20.0) | 1/8 |
| Follicular carcinoma^{b} | 7 (15.6) | 0/7 |
| Poorly differentiated carcinoma | 1 (2.2) | 0/1 |
| Medullary thyroid cancer | 1 (2.2) | 0/1 |

Abbreviation:
NA, not applicable
^{a}Among the Hurthle cell carcinomas, 7 showed capsular invasion and 2 showed vascular invasion. The false-negative case was previously false-negative on the gene expression classifier.²⁰
^{b}Among the follicular carcinomas, 3 showed capsular invasion and 4 were well-differentiated carcinomas not otherwise specified.

TABLE 9
Cytologic Findings and Histopathological Diagnosis in 4 False-
Negative Results on Genomic Sequencing Classification

| | Nodule Size, cm | | Bethesda | Final |
|---|---|---|---|---|
| | Ultrasonographic | Pathological | Cytologic | Histologic |
| Patient No./Sex | Imaging | Examination | Diagnosis | Diagnosis |
| 1/M | 1.1 | 1.2 | III | PTC |
| 2/F | 2.5 | 1.5 | III | PTC |
| 3/F | 3.2 | 3.0 | IV | FVPTC |
| 4/F | 2.9 | 3.5 | IV | HCC-v |

Abbreviations: FVPTC, papillary thyroid cancer follicular variant; HCC-v, Hürthle cell carcinoma, vascular invasion; PTC, papillary thyroid cancer.

Genomic sequence classifier to gene expression classifier comparison on a per-samples basis: 190 Bethesda III/IV primary validation samples yielded both GSC and GEC results (FIG. 5, Table 10). GSC had 99 true negative results; 67 of which were also benign per the GEC, and 32 were GEC suspicious (false positive). GSC had 46 false positive results; 40 of which were also suspicious per the GEC, and 6 were GEC benign (true negative). Of all benign samples (145), GSC reclassified as benign 32 of the GEC's 72 false positive results. Conversely, only 6 of the GEC's 73 true negative results were incorrectly classified as GSC suspicious. The net reclassification of 26 benign nodules to a GSC benign result accounts for the rise in GSC specificity compared to the GEC. GSC had 41 true positive results; 39 of which were also suspicious per the GEC, and 2 were GEC benign (false negative). GSC had 4 false negative results; 3 of which were also benign per the GEC, and 1 was GEC suspicious (true positive). Of all malignant samples (45), GSC reclassified as suspicious 2 of the GEC's 5 false negative results. Conversely, only 1 of the GEC's 40 true positive results were incorrectly classified as GSC benign. The net reclassification of 1 malignant nodules to a GSC suspicious result accounts for the maintained sensitivity of the GSC compared to the GEC.

TABLE 10
Performance comparison between the genomic sequence
classifier and gene expression classifier

| | | | GEC | | | | |
|---|---|---|---|---|---|---|---|
| | | | Histo B | | Histo M | | |
| | | | True | False | True | False | |
| | | | Negative | Positive | Positive | Negative | |
| GSC | Histo B | True Negative | 67 | 32 | | | 99 |
| | | (TN) | | | | | |
| | | False Positive | 6 | 40 | | | 46 |
| | | (FP) | | | | | |
| | Histo M | True Positive | | | 39 | 2 | 41 |
| | | (TP) | | | | | |
| | | False Negative | | | 1 | 3 | 4 |
| | | (FN) | | | | | |
| | | | 73 | 72 | 40 | 5 | 190 |

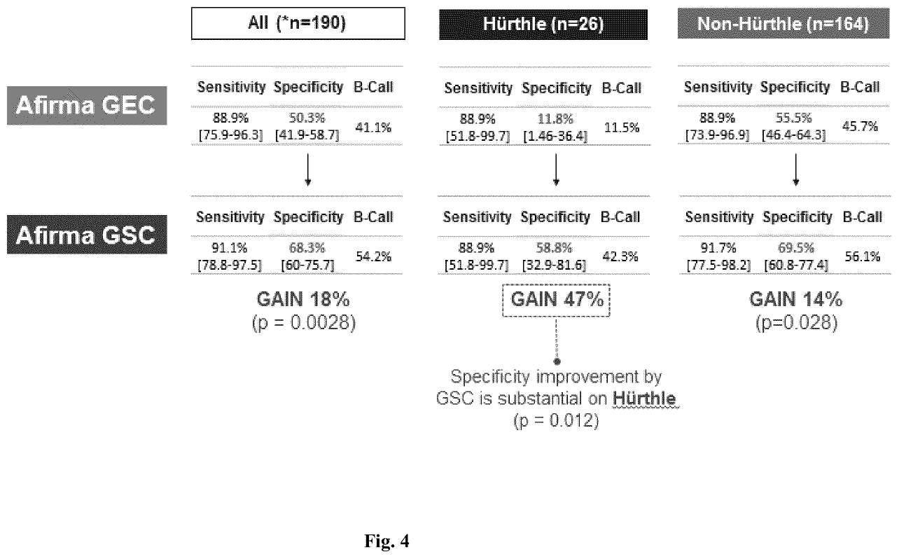

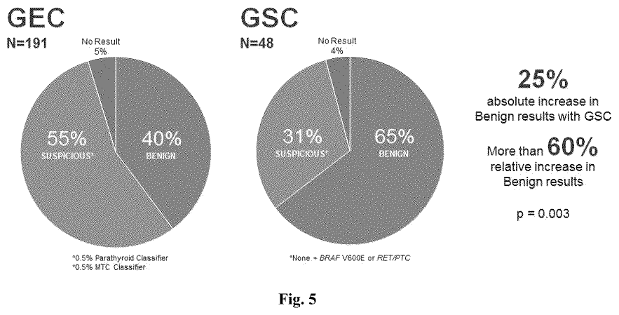

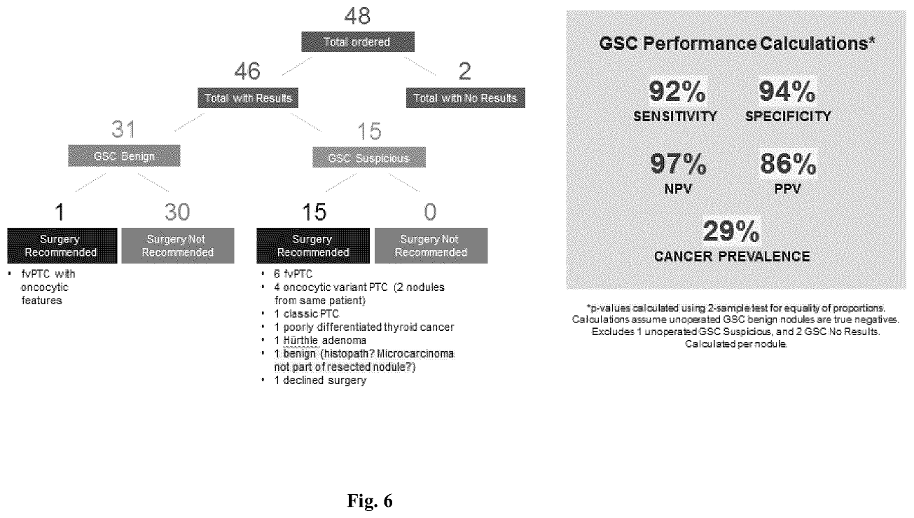

A 2016 meta-analysis reported the risks of malignancy among Bethesda III and IV thyroid nodules to be 17% (95% CI, 11-23) and 25% (95% CI, 20-29), respectively. To safely avoid unnecessary diagnostic surgery among these cytologically indeterminate nodules, a test with a high sensitivity and NPV for malignancy is required. This blinded clinical validation of the GSC in a prospectively collected, representative, universally operated, and histopathologically diagnosed cohort demonstrates the required high NPV across these ranges of cancer prevalence encountered in Bethesda III and IV nodules in clinical practice (FIG. 3). To independently validate the GSC a set of strict blinding and de-identification protocols were implemented that enabled the use of the same FNA samples previously used to validate the GEC. Use of these samples allowed testing of complete and representative sets of nodules with corresponding surgical histology unaffected by the current widespread use of molecular testing to avoid or encourage surgery.

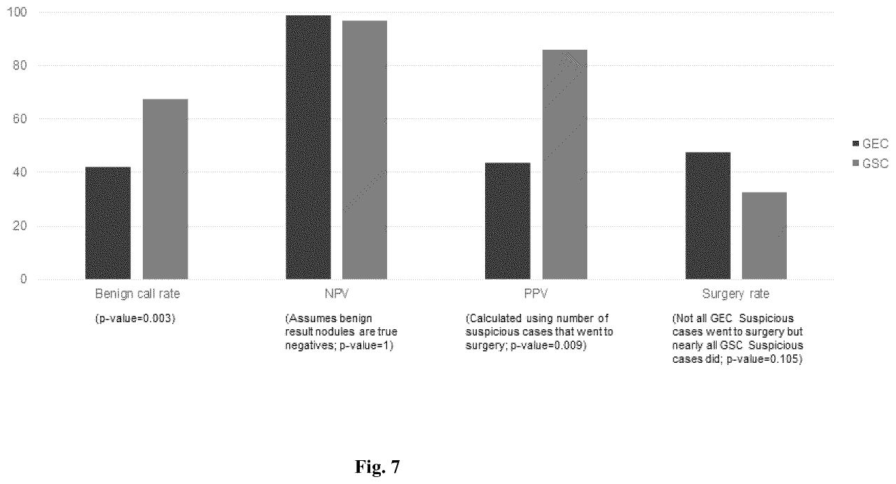

Test sensitivity of the GSC (91%; 95% CI, 79-98) compared with the GEC (89%; 95% CI, 76-96) was maintained, with the point estimate within the counterpart's 95% CI, and the McNemar χ2 test (df=1) on the matched sample set renders a test statistic of 0 (P>0.99). On the other hand, test specificity of the GSC (68%; 95% CI, 60-76) was significantly improved from the GEC (50%; 95% CI, 42-59), with the point estimate outside the counterpart's 95% CI, and the McNemar χ2 test (df=1) on the matched sample set renders a test statistic of 16.447 (P<0.001) (Table 10). In practice, this enhanced performance indicates that among Bethesda III and IV nodules that are histopathologically benign, at least one-third more will receive a benign result using the GSC compared with the GEC (FIG. 5, and FIG. 7). At a cancer prevalence of 24%, more than half of tested patients are projected to receive a GSC benign result, and among GSC suspicious nodules, nearly half are anticipated to have cancer on surgical histology. This increased benign call rate is expected to result in more patients being assigned to active observation as opposed to diagnostic surgery. FIG. 6, for example, illustrates the treatment recommendations to the patients based on the results from Afirma GSC. Given the high cost of surgery in the United States among Medicare and private payers, the increased avoidance of diagnostic surgery because of GSC benign results is expected to further improve cost-effectiveness and reduce surgical complications.

While genomic data has been incorporated in clinical management decisions of multiple medical conditions for more than a decade, progress continues toward understanding the complexities of genomic and non-genomic pathways in the development and behavior of disease. Current evidence suggests that most common diseases are associated with small effects from a large number of genes and that most of these contributions are derived from transcriptionally active portions of the genome. This implies that diseases such as thyroid cancer are unlikely to be accounted for by the effects of a small number of genes. The fact that few genomic variants are associated with 100% penetrance toward malignant histology suggests that a complex interaction of multiple factors ultimately determines the benign or malignant nature of thyroid nodules. As the number of these factors expands, it becomes critical to use machine learning and statistical models to interpret their signals in a trained model to derive an accurate diagnosis.

Hürthle lesions exemplify the challenges inherent in complex biology and the opportunity to harness high dimensional genomic data for predictive model training and subsequent validation. Most Hurthle cell-dominant Bethesda III and IV thyroid nodules have historically undergone surgery given the potential for Hurthle cell carcinoma, yet most have proven to be histologically benign. The GEC identified these samples at a high NPV, but most were categorized as GEC suspicious. Current methods sought to maintain a high NPV while providing more benign results by including 2 dedicated classifiers to work with the core GSC classifier. Among the 26 Hurthle cell adenomas or Hurthle cell carcinomas reported here, the final GSC sensitivity was 88.9% and the specificity was 58.8%; the GEC sensitivity was 88.9% and the specificity was 11.8% among these same neoplasms. Thus, while the overall GSC sensitivity of 91.1% reported here is comparable with that of the GEC (by design), the improved overall GSC specificity of 68.3% results from significantly improved performances among both Hurthle and non-Hurthle specimen types. Given that most histologically benign Hurthle and non-Hurthle specimens are now both identified as GSC benign, GSC testing may further safely reduce unnecessary surgery among both specimen types.

A secondary analysis of 61 Bethesda II, V, or VI samples that also were included in the GEC validation study is included in Table 7. The consistency of these performance metrics within the Bethesda III and IV categories is reassuring and supportive of the findings in the primary analysis.

Methods and systems of the present disclosure may be combined with or modified by other methods or systems, such as, for example, those described in U.S. Pat. No. 8,541,170, U.S. Patent Publication No. 2018/0157789, and U.S. Patent Publication No. 2018/0016642, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for diagnosing thyroid disease in a subject, the method comprising:
(a) providing a DNA sample from a subject;
(b) detecting the presence of one or more polymorphisms selected from the polymorphisms listed in Tables 3-6 or their complement; and
(c) determining whether said subject has or is likely to have a malignant or benign thyroid condition based on the results of step (b).

2. The method of claim 1, wherein the malignant condition is selected from the group consisting of follicular carcinoma, follicular variant of papillary carcinoma, and papillary thyroid carcinoma.

3. The method of claim 1, wherein the benign thyroid condition is selected from the group consisting of follicular adenoma, and nodular hyperplasia.

4. The method of claim 1, wherein the DNA sample provided from said subject is obtained from a sample comprising thyroid tissue.

5. The method of claim 1, wherein the polymorphism comprises a variation in copy number as compared to a normal sample.

6. The method of claim 5, wherein the variation in copy number as compared to a normal sample comprises a deletion.

7. The method of claim 5, wherein the variation in copy number as compared to a normal sample comprises an increase in the copy number.

8. The method of claim 5, wherein the normal sample comprises a sample of DNA from the same subject.

9. The method of claim 5, wherein the normal sample comprises a sample of DNA from a different subject.

10. The method of claim 5, wherein the normal sample comprises a known or generally accepted value.

11. The method of claim 1, wherein the detecting step (b) comprises:
(a) contacting said DNA sample with one or more binding agents that specifically bind to the one or more polymorphisms listed in Tables 3-6, or their complement; and
(b) determining whether said DNA sample specifically binds to said one or more binding agents,
wherein binding of said DNA sample to said one or more binding agents indicates the presence of the polymorphism in said subject.

12. The method of claim 1, wherein the detecting step (b) comprises sequencing of one or more nucleic acid regions comprising the one or more marker regions listed in Tables 3-6 or their complement.

13. The method of claim 1, wherein the detecting step (b) comprises quantifying the amount of DNA comprising the one or more marker regions listed in Tables 3-6 or their complement.

14. The method of claim 13, wherein the quantifying comprises PCR.

15. The method of claim 14, wherein the PCR comprises real-time PCR.

16. The method of claim 13, wherein the quantifying comprises hybridization.

17. The method of claim 1, wherein the method further comprises determining the expression level of one or more genes correlated with follicular adenoma, follicular carcinoma, nodular hyperplasia, follicular variant of papillary carcinoma, or papillary thyroid carcinoma.

18. A composition comprising one or more binding agents that specifically bind to the one or more polymorphisms listed in Tables 3-6, or their complement.

19. A kit for diagnosing thyroid disease in a subject, the kit comprising:
(a) at least one binding agent that specifically binds to the one or more polymorphisms selected from the group consisting of the polymorphisms listed in Tables 3-6, or their complement; and
(b) reagents for detecting binding of said at least one binding agent to a DNA sample from a subject.
